SEQUENCE LISTING (3 sequences)

SEQ ID NO: 1 - DNA, 48 bp, Artificial sequence
  Synthetic polynucleotide
gacttatgatcggataagagtccagccaatgacagatgcctcatagcc

SEQ ID NO: 2 - DNA, 48 bp, Artificial sequence
  Synthetic polynucleotide
ggctatgaggcatcccatgcgttgaggactcttatccgatcataagtc

SEQ ID NO: 3 - RNA, 31 nt, Artificial sequence
  Synthetic polynucleotide
gaguuguaauaacaagggaaaugucauuggc

(19) United States
(12) Patent Application Publication
(10) Pub. No.: US 2023/0040081 A1
(43) Pub. Date: Feb. 9, 2023

(54) VACCINIA VIRAL POLYMERASE-MEDIATED VIRAL REPLICATION

(71) Applicant: IMMUNOLUX INTERNATIONAL CORP., Highland, CA (US)

(72) Inventor: Aladar A. SZALAY, Highland, CA (US)

(21) Appl. No.: 17/784,572

(22) PCT Filed: Dec. 11, 2020
(86) PCT No.: PCT/US20/64624

§ 371 Date: Jun. 10, 2022

Related U.S. Application Data
(60) Provisional application No. 62/946,828, filed on Dec. 11, 2019.

(51) Int. Cl. C07K 16/08; C12N 15/113; A61P 31/20

(52) U.S. Cl. CPC C07K 16/081; C12N 15/1131; A61P 31/20; C12N 2310/18; C12N 2310/11

(57) ABSTRACT

Methods and compositions for regulating activity of a poxvirus viral polymerase by modulating the assembly and/or interaction of one or more subunits of the viral polymerase are described.

### CROSS-REFERENCES TO RELATED APPLICATIONS

This application claims the benefit of U.S. Provisional Application No. 62/946,828, filed Dec. 11, 2019, which is incorporated herein by reference in entirety and for all purposes

### Reference to a “Sequence Listing,” a Table, or a Computer Program Listing Appendix Submitted as an Ascii File

The Sequence Listing written in file 055523-504001WO_SequenceListing_ST25.txt, created Dec. 11, 2020, 4,096 bytes, machine format IBM-PC, MS Windows operating system, is hereby incorporated by reference.

### BACKGROUND

The eukaryotic nucleus contains the machineries for DNA replication and gene transcription. Many viruses rely for their replication and transcription on factors of the host cell and therefore require at least a transient nuclear phase to ensure viral propagation. A remarkable exception amongst eukaryotic DNA viruses are the members of the Poxviridae family, whose replication and transcription are confined to the cytoplasm (Moss, 2013). These processes require virus-encoded factors for the production of mature mRNAs from the viral genome.

The Poxviridae family includes variola virus (smallpox) and vaccinia virus (smallpox vaccine). Although natural smallpox was declared eradicated worldwide in 1980, there remains a risk that the smallpox virus, or a variation of it, could be used as an agent of bioterrorism. In addition, vaccinia virus is being studied as a potential cancer therapy (e.g., as an oncolytic virus).

Thus, it would be beneficial to regulate replication and/or transcription of poxviruses.

### SUMMARY

The instant technology generally relates to methods and compounds for regulating activity of a poxvirus viral polymerase in a cell infected with the poxvirus. In some aspects, regulating the activity of the poxvirus viral polymerase reduces or inhibits transcription of a viral gene(s) by the polymerase.

In an aspect, a method for regulating activity of a poxvirus viral polymerase in a cell infected with the poxvirus is provided. In embodiments, the method includes contacting the cell with a compound that reduces or prevents interaction of the viral polymerase with a glutamine tRNA (tRNA^{Glu}).

In an aspect, a method for treating or preventing infection by poxvirus in a subject in need thereof is provided. In embodiments, the poxvirus includes (or encodes) a viral polymerase and the method includes administering to the subject a compound that reduces or prevents interaction of the viral polymerase with a glutamine tRNA (tRNAGlu).

In an aspect, a method for modulating activity of a poxvirus viral polymerase in a cell infected with the poxvirus is provided. In embodiments, the method includes contacting the cell with glutamine. In embodiments, the glutamine modulates interaction of the viral polymerase with a glutamine tRNA (tRNA^{Glu}). In embodiments, the glutamine may reduce or prevent interaction of the viral polymerase with the tRNA^{Glu}. In embodiments, the glutamine may increase or promote interaction of the viral polymerase with the tRNA^{Glu}.

In an aspect, a method for regulating activity of a poxvirus viral polymerase in a cell infected with the poxvirus is provided. In embodiments, the method includes contacting the cell with a compound that modulates activity of the viral polymerase. In embodiments, the compound reduces or inhibits activity of the viral polymerase. In embodiments, the compound enhances or promotes activity of the viral polymerase. In embodiments, the compound interacts with an active site of the viral polymerase.

In an aspect, a method for treating or preventing infection by poxvirus in a subject in need thereof is provided. In embodiments, the poxvirus includes (or encodes) a viral polymerase, and the method includes administering to the subject a compound that interacts with an active site of the viral polymerase.

In embodiments, the active site includes a binding site for a catalytic metal ion. In embodiments, the catalytic metal ion binding site is a D×D×D site on an Rpo147 subunit, or variant or homologue thereof. In embodiments, the compound reduces or inhibits binding of the catalytic metal ion to the binding site for the catalytic metal ion.

In embodiments, the compound reduces or inhibits interaction of subunit Rpo30 with the active site.

In embodiments, the compound interacts with an active site of a poxvirus capping enzyme.

In embodiments, the compound inhibits or reduces interaction of one or more subunits of the viral polymerase from interacting with the viral polymerase. In embodiments, the one or more subunits of the viral polymerase comprise one or more of: Rpo147, Rpo132, Rpo35, Rpo22, Rpo19, Rpo18, Rpo7, Rpo30, Rap94, a capping enzyme, a termination factor, VETF-1, VETF-s, E11L, tRNAGlu, NPH-1, VTF/CE, and/or any poxvirus polymerase subunit as listed or described in Appendix A and/or Appendix B, or a variant or homologue thereof.

In embodiments, the poxvirus is a variola virus or variant thereof. A variant of the variola virus may be, for example, an engineered or otherwise manipulated virus. For example, the variola virus may have been produces, engineered, and/or manipulated as a bioterrorism agent.

In embodiments, the poxvirus is a vaccinia virus or variant thereof. In embodiments, the vaccinia virus or variant thereof is a smallpox vaccine. In embodiments, the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains. In embodiments, the vaccinia virus is ACAM1000. In embodiments, the vaccinia virus is ACAM2000. In embodiments, the vaccinia virus is a New York City Board of Health strain. In embodiments, the poxvirus is an attenuated virus.

In embodiments, the viral polymerase is a virus-encoded RNA polymerase. In embodiments, the viral polymerase is a virus-encoded multisubunit RNA polymerase (vRNAP).

In embodiments, the compound comprises a small molecule, an antisense RNA, an antibody, an aptamer, or a polypeptide. The compound may be any compound that interacts with the polymerase, such as a subunit, active site, or other component of the polymerase. The compound may inhibit binding of a subunit, active site, or other component of the polymerase to other components of the polymerase, thereby preventing formation of a complete polymerase complex.

In embodiments, the infected cell is a stem cell, immune cell, or cancer cell. In embodiments, the stem cell may be an adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, or any combination thereof.

### BRIEF DESCRIPTION OF THE DRAWINGS

FIG. 1A shows measured total integrated intensity of CV-1 cells overtime during glutamine experiment. The x-axis depicts time post-infection in hours; the y-axis depicts total integrated. Error Bars represent calculated standard error. “+” and “−” represent glutamine presence or absence during the first medium switch, respectively.

FIG. 1B shows measured total integrated intensity of CV-1 cells overtime during glutamine experiment. The x-axis depicts time post-infection in hours; the y-axis depicts total integrated. Error Bars represent calculated standard error. “+” and “−” represent glutamine presence or absence during the second medium switch, respectively.

FIG. 1C shows measured total integrated intensity of CV-1 cells overtime during glutamine experiment. The x-axis depicts time post-infection in hours; the y-axis depicts total integrated. Error Bars represent calculated standard error. “+” and “−” represent glutamine presence or absence during the third medium switch, respectively.

FIG. 2 shows virus titer percentage of each sample compared to sample +/+/+. Error bars represent standard deviation. Statistically significant differences (Student T-test, p<0.05) based on triplicates versus positive control +/+/+ are marked with asterisks.

FIGS. 3A-3C. FIG. 3A shows a cartoon representation of the vRNAP EC. Subunit coloring is as indicated and as in Grimm et al., 2019. Helices are shown as cylinders. Nucleic acids are shown in blue (template strand DNA), cyan (non-template strand DNA, and red (RNA). Metal ions are shown as spheres. FIG. 3B shows close-up view of the active center of vRNAP. Protein and nucleic acids are shown as sticks and colored as in FIG. 3A. The cryo-EM density is shown as gray mesh. The vRNAP EC is in the post-translocated state, and the +1 template base is ready to base pair with an incoming nucleotide. Residues unique to vRNAP as discussed in the test are highlighted in green. FIG. 3C shows schematic depiction of the nucleic acid scaffold used in this study. Individual bases are shown as circles, and the bases are abbreviated as one-letter codes. Bases viable in the EC structure are shown as solid circles, and the invisible bases are shown as hollow circles. The active-site metal A is shown as a pink sphere. vRNAP residues within a distance of 4 Å of the nucleic acids are indicated and colored according to there conservation in S. cerevisiae Pol II. Residues unique to vRNAP as discussed herein are highlighted in green. See also FIGS. 10, 11, and 12.

FIGS. 4A-4B show that nucleic acids replace the Rpo30 C-Terminal tail. FIG. 4A is a cartoon representation of vRNAP in the EC and the comple vRNAP structure (Grimm et al. 2019). The Rpo30 C-tail occupies the hybrid binding site. Subunit coloring is as in FIG. 3. helices are shown as cylinders. Proteins, except for Rpo30, are shown transparently. Nucleic acids are shown in blue (template strand DNA), cyan (non-template strand DNA), and red (RNA). FIG. 4B shows a stick representation of the DNA-RNA hybrid in the vRNAP-EC active site with the Rpo30 C-tail from the complete vRNAP complex (PDB:6RFL) (Grimm et al. 2019) overlaid transparently. Both structures were aligned with the large vRNAP subunit Rpo147.

FIGS. 5A-5C show structure of the vRNAP Co-transcriptional Capping Complex. FIG. 5A: Structure of the vRNAP CCC. (Top) Schematic representation of the D1 and D12 subunits of VTF/CE. (Bottom) Cartoon and surface representation of the vRNAP CCC. vRNAP is shown as gray transparent surface, and CE is shown as cartoon and colored as indicated above. Helices are depicted as cylinders. Nucleic acids are shown in blue (template strand DNA), cyan (non-template strand DNA), and red (RNA). Metal ions are shown as spheres. Parts of the RNA that were not included in the final model are shown as transparent backbone. FIG. 5B: Cryo-EM density for nucleic acids in the CCC. Protein is depicted as cartoon with coloring as in FIG. 5A. The unsharpened cryo-EM density around the nucleic acids is shown as surface and colored around the nucleic acids as in FIG. 5A. The trajectory of the entire RNA can be unambiguously traced. FIG. 5C: Modeled nucleic acids in the CCC shown as stick representation. The parts of the RNA that are likely mobile and scrunched and that were not included in the final model are shown as transparent backbone. Active-site metals are shown as spheres.

FIGS. 6A-6F show a detailed View of vRNAP-CE Interactions and Active Sites. FIG. 6A: Close-up view of the vRNAP-CE interactions around the TP/GT module in side view. Proteins are shown as cartoons and colored as in FIG. 5. The core vRNAP is additionally shown as transparent surface. Subunits Rpo18 and Rpo19 are colored purple and light blue, respectively. FIG. 6B: Close-up view of the vRNAP-CE interaction around the TP/GT module from the opposite side as in FIG. 6A. Depiction and coloring are as in FIG. 6A. FIG. 6C: Close-up view of the vRNAP-CE interactions around the MT/D12 module. Depiction is as in FIG. 6A. Rpo35 is colored in red, and Rpo132 is colored in sand. The Vaccinia-specific Rpo35 region that might interact with the interdomain linker is indicated. Rpo147, Rpo18, and the DNA and RNA were omitted for clarity. FIG. 6D: Sequential arrangement of CE active sites. Back view of the CCC is depicted as in FIG. 5, and proteins are shown transparently. Nucleic acids are shown as sticks, and metal ions are shown as spheres. Parts of the RNA that were not included in the final model are shown as a dashed line. GTP and SAM are shown as sticks. GTP was modeled by the superimposition of the CE crystal structure (PDB: 4CKB) (Kyrieleis et al., 2014) with its TP/GT module. Active sites are labeled with numbers according to their order of action on the RNA substrate. FIG. 6E: Close-up view of the CE TPase active site. Residues lining the inside of the catalytic beta-barrel and the RNA are shown as sticks. The catalytic metal is shown as a sphere. FIG. 6F: Close-up view of the CE MTase active site. The SAM cofactor is shown as sticks, and cryo-EM density is shown as gray mesh. Residues within 4A of the SAM molecule are shown as sticks.

FIG. 7 shows transitions from the Complete vRNAP Complex to the CCC. (Top) Structure of the complete vRNAP complex (Grimm et al., 2019). Proteins are depicted as a schematic surface. vRNAP is colored in gray. Rap94, NPH-I, VETF, E11, and rRNA are colored in forest green, red, purple, yellow and orange, respectively. Proteins that are likely to dissociate or rearrange upon formation of the CCC are shown transparently. The Rpo147 C-tail is colored in teal and highlighted. Arrows indicate the transitions that must occur upon formation of the CCC. (Bottom) Structure of the CCC colored as in FIG. 5. The Rpo147 C-tail, which adopts a helical conformation in the CCC, is highlighted.

FIG. 8 shows growing RNA Displaces the Rap94 B-Homology Region. (Top) Schematic depiction of Rap94 and S. cerevisiae TFIIBs with domains and boundaries indicated. (Bottom) Comparison of the active center cleft of the complete vRNAP complex and the S. cerevisiae Pol II initially transcribing complex (PDB: 4BBS) (Sainsbury et al., 2013). Proteins and nucleic acids are shown as cartoon representations and are colored as indicated. vRNAP and Pol II elements are colored as in Grimm et al. (2019) and Sainsbury et al. (2013). The nucleic acid structure from the CCC was overlaid with the complete vRNAP complex by the alignment of the large subunits Rpo147 and is shown transparently. The circle indicates the region where clashes occur. The Rudder loop in the polymerase, which interacts with the B-linker and B-reader in Pol II, adopts a different conformation in vRNAP than in Pol II.

FIG. 9 shows comparison of Complete vRNAP Complex and S. cerevisiae Initially Transcribing Complex. The vRNAP-Rap94 complex has a similar topology as the Pol II-TFIIB complex. (Left) vRNAP-Rap94 complex in the complex vRNAP complex (Grimm et al., 2019). All other proteins were omitted for clarity. vRNAP is colored in gray, and Rap94 is colored in green, both with shadings as in FIG. 6. Domain 2 and the CTD are shown transparently. Proteins are depicted as cartoon representations with cylindrical helices. (Right) Structure of the S. cerevisiae Pol II initially transcribing complex (PDB: 4BBS) (Sainsbury et al., 2013). Depiction is as on the left, and nucleic acids are colored as in FIG. 3.

FIGS. 10A-10B show purification of Transcribing vRNAP Complexes, Related to FIGS. 3 and 5. FIG. 10A: Schematic representation of the purification strategy of vRNAP bound to a DNA/RNA scaffold. FIG. 10B: Representative 10%-30% sucrose density gradient of affinity-purified vRNAP complexes bound to a DNA/RNA scaffold. Proteins and nucleic acids of individual fractions were separated by SDS-PAGE and visualized by silver staining (top) and EtBr staining (bottom), respectively. The fractions 15 and 16 were pooled and used for cryo-EM analysis.

FIGS. 11A-11C show structure determination of vRNAP EC and CCC, Related to FIGS. 3 and 5. FIG. 11A: Representative cryo-EM micrograph from the dataset. FIG. 11B: Best aligning classes of unsupervised 2D classification in Relion. FIG. 11C: Workflow for structure determination of the EC and CCC. Unsharpened final densities are shown colored according to their subunit composition as in FIG. 7.

FIGS. 12A-12E show Cryo-EM Structure Statistics and Information, Related to FIGS. 3 and 5. FIG. 12A: Fourier shell correlation plots for the EC. CCC and core vRNAP structures. FIG. 12B: Comparison of cryo-EM densities of the EC, CCC and core vRNAP reconstructions determined here. Densities are shown transparently in blue (EC), red (CCC) or green (core vRNAP) with the model of the Rpo147 funnel helices shown as sticks. FIG. 12C: Angular distribution and local resolution of the CCC reconstruction. FIG. 12D: Angular distribution and local resolution of the EC reconstruction. FIG. 12E: Angular distribution aid local resolution of the core vRNAP reconstruction.

FIGS. 13A-13D show details of Capping Enzyme, Related to FIGS. 5 and 6. FIG. 13A: Comparison of the CCC structure to the CE crystal structure (PDB ID 4CKB) (Kyrieleis et al., 2014). Depiction slightly rotated from the top view shown in FIG. 5. The crystal structure was aligned to the CCC structure with the TP/GT module and is shown transparently. The MT/D12 module adopts a different orientation relative to the TP/GT module than in the crystal structure. FIG. 13B: Back view of the CCC. Protein and nucleic acids are depicted as cartoon with cylindrical helices and colored as in FIG. 5. Parts of the RNA not included in the final model are shown as transparent backbone. The CE active sites are indicated. The bound S-adenosylmethionine cofactor is shown as sticks in the MTase active site. FIG. 13C: Close-up view of the TPase active site. Coloring as in FIG. 5. Residues lining the inside of the beta barrel and the RNA are shown as sticks. The active site metal is shown as sphere. FIG. 13D: Comparison to the S. cerevisiae Cet1 structure. The TPase active site in the CCC is superimposed with the Cet1 crystal structure (Lima et al., 1999) and the homologous catalytic glutamate residues are shown as sticks. Cet1 is shown transparently. The sulfate ion proposed to mimic the leaving gamma-phosphate in the crystal structure is indicated.

FIGS. 14A-14B show comparison of the CE Interdomain Linker in the Complete vRNAP Complex and the CCC, Related to FIG. 7. FIG. 14A: Structure of the CE interdomain linker (residues 529-560) in the complete vRNAP complex (Grimm et al., 2019). Proteins are shown transparently in cartoon representation with coloring as in FIG. 5. This linker is colored in teal and highlighted. In the complete vRNAP complex, the linker is fully ordered and shifted toward the MTase active site. Residue Y555 occupies the binding site of the SAM cofactor. The SAH cofactor bound in the CE crystal structure (PDB ID 4CKB) (Kyrieleis et al., 2014) is modeled based on its location in the crystal structuren and shown as transparent slicks to illustrate the overlap. FIG. 14B: Structure of the CE interdomain linker (residues 529-560) in the CCC. Depiction as in FIG. 14A. The linker is only partially ordered in the CCC structure and in previous crystal structures (Kyrieleis et al., 2014; De la Pena et al., 2007), but the backbone density in the CCC reconstruction clearly indicates an identical trajectory as in these crystal structures. In these structures, the backbone and Y555 are positioned away from the SAM binding site to allow cofactor binding. The region 543-547 of the interdomain linker is clearly visible in the EM density and is positioned in immediate vicinity of the Vaccinia-specific part of Rpo35 (residues 147-185), and K546 of D1 may form ionic interactions with D153 or E152 in Rpo35.

FIG. 15 shows sequence comparison of Rap94 and S. cerevisiae TFIIB, Related to FIG. 8. Structure-based alignment of the Rap94 B-homology region and S. cerevisiae TFIIB. Residues coordinating the structural Zn ion in the B-ribbon are colored in pink. The region in the TFIIB B-reader conserved between species is indicated and not conserved in Rap94. Invariant residues are colored in blue and conserved residues in light blue. The alignment was generated with MSAProbs (Liu et al., 2010) within the MPI Bioinformatics Toolkit (Zimmermann et al., 2018) using Aline (Bond and Schüttelkopf, 2009) and manually edited by comparison to the S. cerevisiae Pol II ITC structure (PDB 4BBS) (Sainsbury et al., 2013).

FIGS. 16A-16D show Rap94 Is Not Present in the EC or CCC, Related to FIGS. 7 and 8. FIG. 16A: The unsharpened cryo-EM reconstruction of the vRNAP EC is shown as transparent blue surface with the EC model shown as cartoon and colored as in FIG. 3. The binding sites of Rap94 domains in the core and complete vRNAP complexes (Grimm et al., 2019) are indicated. No density for Rap94 is observed. FIG. 16B: The unsharpened cryo-EM reconstruction of the particle population lacking nucleic acids in our dataset is shown as transparent gray surface with the vRNAP-Rap94 model from the complete vRNAP complex shown as cartoon and colored as in FIG. 3. Rap94 is colored in forest great. Clear density is visible for the Rap94 Domain 2, the B-homology domain and the CTD, with only the NTD lacking density. FIG. 16C: The active center cleft is occupied by nucleic add in the EC. Close up view of the active center deft in the EC depicted as in FIG. 16A. Density corresponding to nucleic acids is shown as solid surface and colored as in FIG. 5B. FIG. 16D: The Rpo30 C-tail occupies the active center cleft in the particle population lacking nucleic acids. Close up view of the active center cleft of the particle population lacking nucleic acids depicted as in FIG. 16B. Density corresponding to the Rpo30 C-tail is shown as solid surface aid colored in orange.

FIGS. 17A-17D show purification and characterization of Vaccinia Virus RNA Polymerase Complexes. FIG. 17A: Purification of Rpo132 and its associated proteins from GLV-1h439-infected cells using anti-FLAG affinity chromatography. Mock purification was performed from cells infected with untagged GLV-1h68. Specific proteins from the GLV-1h493 elution were resolved on SDS gels and identified by mass spectrometry. FIG. 17B: Anti-FLAG eluate from cell extracts infected with GLV-1h439 was separated on a 10%-30% sucrose gradient and proteins visualized by silver staining on SDS-PAGE. FIG. 17C: RNA extension assay with a nucleic acid scaffold mimicking an elongation complex transcription bubble. FIG. 17D: Transcription assay with a linearized pSB24 template containing a Vaccinia virus early promoter and early gene termination signal.

FIGS. 18A-18C show the structure of Core Vaccinia RNAP. FIG. 18A: Schematic depiction of vRNAP subunits. Functional domains are annotated based on structure-based sequence alignment with S. cerevisiae RNA Pol II (Armache et al., 2005; Cramer et al., 2001). Regions not visible in the core v RNAP structure are shown transparently. FIG. 18B: Structure of the core Vaccinia RNA polymerase enzyme. The protein is shown in cartoon depiction, with helices depicted as cylinders. Subunits are colored as in FIG. 18A. The active site metal A and bound structural zinc ions are shown as spheres. FIG. 18C: Cartoon depiction of Vaccinia RNAP subunits with structural details shown. Rpo147 and Rpo132 domains are colored as indicated in FIG. 18A. The location of the subunits in the enzyme is indicated schematically.

FIGS. 19A-19B show a comparison of Vaccinia RNA Polymerase to S. cerevisiae Pol II. FIG. 19A: Comparison of subunit composition between core vRNAP and S. cerevisiae Pol II (PDB: 1WCM) (Armache et al., 2005). The enzymes are depicted in schematic surface representation. Homologous subunits are indicated in the table and colored accordingly. FIG. 19B: Detailed comparison of core vRNAP (left) and S. cerevisiae Pol II (right) (PDB ID: 1WCM)(Armache et al., 2005). The largely conserved core is depicted as schematic surface in gray, and the differing regions are depicted as cartoon. Regions specific to vRNAP are shown in green and regions specific to Pol II in red. Regions located at the back of the enzyme are labeled transparently.

FIGS. 20A-20B show structure of the Complete vRNAP Complex. FIG. 20A: Schematic depiction of the additional Vaccinia transcription factors VTF/CE, VETF-I, E11, and NPH-I contained in the complete vRNAP complex with domains indicated. Rpo30 and Rap94 are also present in the core vRNAP complex. FIG. 20B: Overview of the complete vRNAP model, color coding as in FIG. 20A. vRNAP is shown in gray. The orientation of the view in the left panel is related to the view in the left panel of FIG. 18B by an approximately 30° rotation counter-clockwise around the viewing axis followed by an approximately 30° rotation counter-clockwise vertical rotation. The protein is shown in cartoon depiction, with helices depicted as cylinders.

FIGS. 21A-21B show Rap94 and Its Role in the Complete vRNAP Complex. FIG. 21A: Location of Rap94 in the complete vRNAP structure. The whole model is shown as transparent gray solvent accessible surface with Rap94 shown as solid cartoon. The active site metal A is shown as sphere. FIG. 21B: Details of the Rpol 47 C-tail and the Rap94 linker 2 (L2). These two elements are shown in worm mode and the rest of the model as solvent accessible surface. The Rpol 47 C-tail was visible as a diffuse corridor in the cryo-EM density and was manually modeled as Ca trace for this figure. The quality of the density for this element did not allow assignment of side chains; therefore, this stretch is omitted in the deposited model. FIG. 21C: The extended Rap94 linker 3 (L3, shown as worm) connects the B-cyclin domain to the CTD and binds into a cleft on the cRNAP core. The model except for Rap94-L3 and the Rpo147 C-tail is shown as solvent accessible surface. FIG. 21D: Close-up view of the CEC and its interactions with VTF/CE and the NPH-I helicase module. Proteins are shown as carbon with coloring as in FIG. 20. FIG. 21E: Details of the E11-Rap94 interactions. FIG. 21F: Details of the Rap94 domain 2 interactions. FIG. 21G: Comparison of the Rap94 B-homology region (top) to the corresponding elements of yeast TFIIB (PDB ID 4BBR)(Sainsbury et al., 2013) (bottom).

FIGS. 22A-22B show structure and interactions of Subunit Rpo30. FIG. 22A: Comparison of Vaccinia Rpo30 and S. cerevisiae TFIIS. The proteins are depicted schematically with domains indicated. The position of Rpo30 on the core vRNAP complex is shown on the left, with the rest of the enzyme shown as transparent surface representation with coloring as in FIG. 18A. The position of TFIIS in the Pol II reactivation intermediate complex (PDB ID: 3PO3) (Cheung and Cramer, 2011) is shown on the right, with the rest of the enzyme shown as transparent surface representation. FIG. 22B: Cross section through the solvent-accessible surface of the complete vRNAP complex model in the area of the active center cleft. The phosphorylated C-tail of Rpo30 is shown in orange as sticks and the phosphate-moieties shown as purple spheres. The Rap94 B-reader is shown as green worm.

FIGS. 23A-23D show interactions of NPH-I and VETF in the Complete vRNAP Complex. FIG. 23A: Location of VETF, NPH-I, E11, and tRNAGIn in complete vRNAP. The whole model is shown as transparent gray solvent accessible surface with the factors shown as solid cartoon models. Color coding as in FIG. 20. FIG. 23B: Details of the NPH-I fold and location of its helicase motifs (left). Comparison to INO80 (right) (PDB 6FHS) (Eustermann et al., 2018). Corresponding regions are colored identically. FIG. 23C: Details if the NPH-I interactions with the tRNA anticodon loop. FIG. 23D: Details of the VETF-I fold and its tRNA interactions. Disulfide bridges we shown as sticks.

FIGS. 24A-24D show purification and activity of vRNAP Complexes, Related to FIG. 17. FIG. 24A: Schematic representation of modified Vaccinia virus genes. A DNA fragment encoding a HA-FLAG-tag was fused in GLV-1h439 to the 3′ end of A24R, allowing the expression of C-terminally tagged Rpo132. FIG. 24B: Replication of GLV-1h439 in comparison to its parental virus GLV-1 h68. Virus titer was determined for the indicated time points from infected cells and cell culture supernatant, respectively. FIG. 24C: Schematic representation of the purification strategy. FIG. 24D: Scheme of the pSB24 template (top) and nucleic-acid scaffold with RNA in red, template DNA it blue, and non-template chain in light pink (bottom) as used for the transcription assays in FIGS. 17C and 17D.

FIGS. 25A-25H show structure determination of Core vRNAP, Related to FIG. 18. FIG. 25A: Exemplary cryo-EM micrograph of the core vRNAP dataset. FIG. 25B: The 32 best aligning class averages from unsupervised 2D classification. FIG. 25C: Cryo-EM processing workflow for structure determination. FIG. 25D: Focused classification and refinement workflow for improved local maps. FIG. 25E: Fourier Shell Correlation (FSC)-plots for cryo-EM reconstructions used. FIG. 25F: Angular distribution plot for the global reconstruction of core vRNAP. FIG. 25G: Local resolution estimation for the global reconstruction of core vRNAP as implemented in Relion. FIG. 25H: Bis(sulfosuccinimidyl)suberate (BS3) crosslinks identified by mass spectrometry used for positioning of Rap94 domains. (Left) Overview of the core vRNAP structure with regions where strong crosslinks occurred indicated. (Indent 1-3) Proteins are shown in cartoon representation with coloring as in FIG. 18. Crosslinked lysine residues are shown as sticks. Selected strong crosslinks are shown as lines.

FIGS. 26A-26B show Structure-Based Sequence Alignment of Rpo147 and S. cerevisae Rpb1, Related to FIG. 19. FIG. 26A: Schematic depiction of Vaccinia Rpo147 and the homologous S. cerevisiae Pol II subunit Rpb1 with domains indicated. Insertions and deletions are indicated by connecting lines, with differing regions shown with dashed lines. Regions with differing fold are indicated by crossed connecting lines. FIG. 26B: Structure-based sequence alignment with secondary structure elements depicted and colored according to domains as in FIGS. 18A and 18C. Sheet regions are shown as arrows, helical region as cylinders. Invariant residues are colored in dark blue and conserved residues in light blue. Regions differing in fold are colored in green (vRNAP-specific) and red (Pol II-specific). The alignment was generated with MSAProbs (Liu et al., 2010) within the MPI Bioinformatics Toolkit (Zimmermann et al., 2018), visualized using Aline (Bond and Schûttelkopf, 2009) and manually edited by comparison to the S. cerevisiae Pol II structure (PDB1WCM) (Armache et al., 2005). In Rpo147, helices α8 and α9 in the polymerase clamp core domain are shortened. Helices α27, α28, α32 and α34, which are located in the foot domain of Rpb1, are absent. The jaw domain is substantially reduced, lacking Rpb1 regions 1158-1188 and 1245-1253.

FIGS. 27A-27B show Structure-Based Sequence Alignment of Rpo132 and S. cerevisae Rpb2, Related to FIG. 19. FIG. 27A: Schematic depiction of Vaccinia Rpo132 and the homologous S. cerevisiae Pol II subunit Rpb2 with domains indicated. Insertions and deletions are indicated by connecting lines, with differing regions shown with dashed lines. Regions with differing fold are indicated by crossed connecting lines. FIG. 27B: Structure-based sequence alignment with secondary structure elements depicted and colored according to domains as in FIGS. 18A and 18C. Sheet regions are shown as arrows, helical region as cylinders. Invariant residues are colored in dark blue and conserved residues in light blue. Regions differing in fold are colored in green (vRNAP-specific) and red (Pol II-specific). The alignment was generated with MSAProbs (Liu et al., 2010) within the MPI Bioinformatics Toolkit (Zimmermann et al., 2018), visualized using Aline (Bond and Schûttelkopf, 2009) and manually edited by comparison to the S. cerevisiae Pol II structure (PDB 1WCM)(Armache et al., 2005). Helices α7 and α8 in the lobe domain are extended in the Rpo132. In the protrusion domain, the region between α11 and α12 differs between the yeast and viral proteins. The most prominent differences are located in the external domains, in particular in the regions between β16 and β17, α16 and α17, and between α19 and β24. The region following β28 (res. 784-797), which contacts upstream DNA in the yeast Pol II (Barnes et al., 2015), is reduced and adopts a different conformation in the viral enzyme.

FIGS. 28A-28B show Structure-Based Sequence Alignment of Rpo35, Rpo22, Rpo19, Rpo18, and Rpo7 with Corresponding S. cerevisae Pol II Subunits, Related to FIG. 19. Structure-based sequence alignments with secondary structure elements depicted and colored according to domains as in FIG. 19. Sheet regions are shown as arrows, helical region as cylinders. Invariant residues are colored in dark blue and conserved residues in light blue. Regions differing in fold are colored in green (vRNAP-specific) and red (Pol II-specific). The alignment was generated with MSAProbs (Liu et al., 2010) within the MPIBioinformatics Toolkit (Zimmermann et al., 2018), visualized using Aline (Bond and Schüttelkopf, 2009) and manually edited by comparison to the S. cerevisiae Pol II structure (PDB 1WCM) (Armache et al., 2005). FIG. 28A: Schematic depiction of Vaccinia Rpo35 and Rpo7 and the homologous S. cerevisiae Pol II subunits Rpb3, Rpb11 and Rpb10 with domains indicated and structure-based sequence alignment between the proteins. Insertions and deletions are indicated by connecting lines, with differing regions shown with dashed lines. Regions with differing fold are indicated by crossed connecting lines. The region resembling the non-conserved domain of Rpb3 responsible for interactions with Rpb10 and Rpb12 is reduced in Rpo35, with the Zn-binding motif lacking altogether. FIG. 28B: Schematic depiction of Vaccinia Rpo22, Rpo19 and Rpo18 and the homologous S. cerevisiae Pol II subunits Rpb5, Rpb6 and Rpb7 with domains indicated and structure-based sequence alignments. Depiction as in FIG. 28A. Like Rpb7, Rpo18 binds to the polymerase core via its K1 helical turn and its tip loop in the amino terminal tip domain. These elements form a wedge between the N-terminal region of Rpo147, the switch 5 region, the Rpo132 anchor, and helix al of Rpo19, all of which are conserved between Vaccinia and Pol II. The Rpo18 tip domain may therefore restrict movement of the clamp, as proposed for Rpb7 in Pol II (Armache et al., 2003). The C-terminal domain of Rpo19 forms a β-barrel-like structure but appears tilted toward the polymerase body compared to Rpb4/7.

FIGS. 29A-29F show Structure Determination of Complete vRNAP, Related to FIG. 20. FIG. 29A: Exemplary cryo-EM micrograph of the complete vRNAP complex dataset. FIG. 29B: Selected Class averages from unsupervised 2D classification in Relion. FIG. 29C: Cryo-EM processing workflow for structure determination. FIG. 29D: Local resolution estimates mapped to the cryo EM density isosurface representation. FIG. 29E: Angular particle orientation map. FIG. 29F: Fourier Shell Correlation (FSC)-plot.

FIGS. 30A-30C show Sequence Alignment of Rpo30 and S. cerevisiae TFIIS aid Structural Details of NPH-I and E11 (Related to FIGS. 20, 21 and 22). FIG. 30A: Structure-based sequence alignment of Rpo30 and S. cerevisiae TFIIS with secondary structure elements depicted and colored according to domains as in FIG. 22. Sheet regions are shown as arrows, helical region as cylinders. Invariant residues are colored in dark blue and conserved residues in light blue. Regions differing in fold are colored in green (vRNAP-specific) and red (Pol II-specific). The alignment was generated with MSAProbs (Liu et al., 2010) within the MPI Bioinformatics Toolkit (Zimmermann et al., 2018), visualized using Aline (Bond and Schûttelkopf, 2009) and manually edited by comparison to the S. cerevisiae. Pol II structure (PDB 1WCM) (Armache et al., 2005). The Zink-binding regions are highlighted in pink and the conserved acidic residues of TFIIS that enter the Pol II active site (DEP motif) are highlighted in green. FIG. 30B: Fold and topology of the E11 crystal structure. Topology (left). Fold and secondary structure elements in cartoon style (right). The two protomers of the homodimer are in orange and yellow, respectively. FIG. 30C: Comparison of the ATPase domains of NPH-I to those of the chromatin remodelers INO80 (PDB 6FHS)(Eustermann et al., 2018) and SNF2 (from PDB ID SXOX) (Liu et al., 2017). The characteristic structural elements are color-coded and labeled.

FIGS. 31A-31C show structure of the vaccinia pre-initiation complex (PIC). FIG. 31A: Overall structure of the PIC in two orthogonal views. The core polymerase is depicted in grey. FIG. 31B: Domain structure of VETFs, VETF1, NPH-I and Rap94. FIG. 31C: Transparent iso-surface of the DNA cryo-EM density, filtered by Gaussian blur with 1.5σ standard deviation, and DNA model are shown in cartoon style. Approximated helix axes of the different duplex DNA sections are indicated, and the translation of the helix axes of the two duplex DNA regions adjacent to the initially melted region (IMR) is denoted. This view is rotated by 20° relative to FIG. 31A.

FIGS. 32A-32E show structure of the VETF heterodimer. FIG. 32A: Two views of VETF with the bound promoter within the PIC are displayed. For easier visualization, the core polymerase is hidden. FIG. 32B: VETF1 CRBD binding to the upstream critical promoter region. Disulfide bridges are depicted as stick model. FIG. 32C: Details of the VETF1 CRBD promoter interaction. The model is depicted in stick-representation, base pairs are numbered relative to the transcription start site (TSS). Only bases for the non-template strand are labelled, the template strand is sequence complementary. Contact between Tyr367 and thymidine bases at positions −18 and −17 are displayed as transparent van-der-Waals surface. The protein-DNA H-bond network is depicted as dotted yellow lines. FIG. 32D: Schematic representation of the sequence-specific interactions of the CRBD reader. The critical region consensus sequence is depicted according to Yang et al. FIG. 32E: Detailed view of VETFs binding to the downstream promoter.

FIGS. 33A-33B show comparison of the TBP-like domain from vaccinia VETF1 with yeast TBP. FIG. 33A: The TBPLD of VETF1 in two orthogonal views. Residues intercalating between the nucleobases are depicted as stick model. FIG. 33B: Structure of the yeast TBP protein bound to a synthetic TATA-box hairpin DNA oligomer41 (PDB 1YTB) in two orthogonal views corresponding to the protein orientation of the VETF1 TBPLD as seen in FIG. 33A.

FIGS. 34A-34C show transition of complete vRNAP to the PIC, and a model for early promoter recognition and opening: FIG. 34A: Complete vRNAP residual density (EMD 4868, grey transparent isosurface) docked with the VETF1 structure and shown along with the complete vRNAP model (PDB 6RFL) in cartoon representation (color code as in FIGS. 31-33 and as in Grimm et al. for the complete vRNAP-specific factors). The predominantly disordered interface of VETF1 to the tRNA aminoacyl stem is marked with an orange dotted line. FIG. 34B: Schematic representation of vaccinia early promoter recognition and opening mechanism (Color code as in FIG. 32). FIG. 34C: Schematic representation of the reconfiguration of complete vRNAP to the PIC.

FIGS. 35A-35D show complex reconstitution and purification. FIG. 35A: Vaccinia virus consensus sequences of early promoter (upper panel). Schematic representation of the DNA scaffold used for reconstitution assays. The scaffold consists of the critical region of the early promoter (CR), a bubble region including the transcription start site (+1) and a G-less template cassette. FIG. 35B: Protein composition of the isolated complete vRNAP as determined by SDS gel electrophoresis (left panel). Complete vRNAP-catalyzed in vitro run-of transcription from a linearized plasmid template containing the vaccinia virus early promoter. FIG. 35C: Left panel: vRNAP binding to the [32P]-labelled promoter DNA scaffold (see FIG. 35A) analyzed by native gel electrophoresis and autoradiography. Indicated amounts of vRNAP was incubated with the DNA scaffold in the presence (lanes 2-4) or absence (lanes 5-7) of NTPs (1 mM each). vRNAP was omitted from control reaction in lane 1. Right panel: Formation of vRNAP/DNA complex is dependent on ATP and UTP. The reaction mixture contained 4 pmol RNA polymerase, the indicated NTP mixture or the ATP analogue AMP-PNP (1 mM each). The reaction was analyzed by native gel electrophoresis and autoradiography. FIG. 35D: Reconstitution and preparative purification of vRNAP-promoter complexes. Approx. 500 pmol affinity-purified complete vRNAP was incubated with a 60-fold molar excess of the DNA scaffold (FIG. 35A) in the presence of 1 mM ATP/UTP mixture and separated by gradient centrifugation. Fractions 13-16 were pooled and used for cryo-EM studies.

FIGS. 36A-36F show cryo-EM reconstruction. FIG. 36A: Classification and refinement scheme. FIG. 36B: Local resolution mapped to the consensus reconstruction density iso-surface (only a mild B-factor sharpening of −10 Å² was applied). FIG. 36C: Masked VETF and DNA region after multibody refinement. FIG. 36D: FSC curves for consensus and multibody refinements. FIG. 36E: Orientation plots referring to the consensus reconstruction in FIG. 36B. FIG. 36F: Selected views of the final, B-factor sharpened (−60 Å²) cryo-EM density isosurface overlaid with the model.

FIG. 37 shows upstream promoter contacts to the core vRNAP. Detailed view of the upstream promoter contacts to the core vRNAP in cartoon representation. The lobe region contacting the DNA is indicated with a rose dotted line. Compare also FIG. 38A.

FIGS. 38A-38C show DNA contacts in the PIC. Transparent iso-surface of the cryo-EM density for the bound DNA, filtered by a Gaussian blur to 1.5σ standard deviation. The model is shown in cartoon style and the initially melted region (IMR) is indicated. FIG. 38A: Top view of the PIC with VETF removed (top view) and vRNAP core shown as solvent accessible surface. The clamp head and lobe are marked on the molecular surface by a rose dotted line, respectively. FIG. 38B: Front view of the PIC with core removed (front view) and VETF shown in cartoon representation. FIG. 38C: PIC with vRNAP removed shown in cartoon view turned by roughly 90° relative to FIG. 38B. and slightly optimized for clarity. Aliphatic residues intercalating into the DNA base plane are shown as stick model.

FIGS. 39A-39B show VETFs and SSL2. FIG. 39A: Cartoon model of VETFs and downstream DNA with superposed ideal B DNA in transparent grey. The respective helix axes are indicated and Phe 271 is depicted in stick representation. FIG. 39B: A depiction of the promoter-bound yeast XPB homologue SSL2 from the yeast PIC bound to TFIIH and core mediator (PDB:5oqm) analogous to FIG. 39A. The axis of the bent, bound DNA (blue) is indicated similarly. Both (referring to FIG. 39A and FIG. 39B) arms of the respective DNA helix axis bend angles lie approximately in the paper plane.

FIG. 40 shows comparison of Vaccinia NPH-I and VETFs with structurally related helicases. Color code according to common structural elements.

FIGS. 41A-41B show comparison of the vaccinia PIC to the Pol II PIC. FIG. 41A: Vaccinia PIC model in cartoon representation as shown in FIG. 31A, front view. FIG. 41B: Pol II core PIC model (PDB 5IY6) in cartoon representation and oriented by superposition of the Pol II core polymerase with the core vRNAP of the vaccinia PIC. Elements identified as functionally, architecturally or structurally corresponding are colored according to the scheme used for the vaccinia PIC throughout Example 4 herein.

FIGS. 42A-42B show structure of the late PIC. FIG. 42A: Model of the 1PIC with density for the bound DNA oligomer shown as a blue surface, for the phosphor-peptide domain (PPD) in transparent gold. FIG. 42B: Domain structure of the bound transcription factors. Disordered regions are marked by hatched boxes.

FIGS. 43A-43B show three structures of initially transcribing complexes. FIG. 43A: Model of the ITC state 1 shown with overlay of the downstream DNA from state 2 and state 3. FIG. 43B: Domain structure of the bound transcription factors. Disordered regions are marked by hatched boxes.

FIGS. 44A-44D show structure of the late ITC. FIG. 44A: Model of the 1ITC in two orthogonal views. FIG. 44B: Domain structure of the bound transcription factors. Disordered regions are marked by hatched boxes. FIG. 44C: Structure of the eukaryotic transcription-coupled repair (TCR) initiation complex, orientation as in FIG. 44A, left view. FIG. 44D: Detailed view of NPH-I bound to the upstream promoter DNA.

FIGS. 45A-45B show promoter melting, bubble stabilization and initiation mechanism. FIG. 45A: Promoter escape mechanism and bubble stabilization. FIG. 45B: Clamp closure in different vRNAP complexes.

FIGS. 46A-46D show cryo EM reconstruction of 1PIC and 1ITC. FIG. 46A: classification and refinement scheme. FIG. 46B: Local resolution mapped to the reconstruction density isosurface. FIG. 46C: FSC plots for consensus refinement and the separate bodies of the multibody (MB) refinement. FIG. 46D: Orientation plots referring to the reconstructions in FIG. 46B.

FIGS. 47A-47D show cryo EM reconstruction of 1PIC. FIG. 47A: classification and refinement scheme. FIG. 47B: Local resolution mapped to the reconstruction density isosurface. FIG. 47C: FSC plots for 1PIC and ITC1-3. FIG. 47D: Orientation plot referring to the reconstructions in b.

FIG. 48 shows vRNAP clamp closure in different vRNAP states. Clamp closure plotted as Cα distance from clamp residue Rpo147 (Lys242) to lobe residue Rpo132 (Glu294).

FIG. 49 shows transcription bubble in the 1ITC. A zoomed view into the active site region is depicted for the ITC1 structure. The base at the active site is indicated relative to the TSS.

FIG. 50 shows transcription bubble in the 1ITC. A zoomed view into the active site region is depicted. Disordered regions of the template and non-template strand are shown as dotted lines. The start and end positions of the melted promoter and the base at the active site are numbered relative to the TSS.

FIGS. 51A-51B show remodelling of Rap94 in the 1ITC complex. FIG. 51A: Relocation of the B-cyclin domain. The 1ITC complex is shown in cartoon style, overlaid with the B-cyclin domain from the 1PIC structure as transparent solvent accessible surface. The relocation is indicated by a magenta arrow. FIG. 51B: Relocation of the B-ribbon domain. The 1ITC complex is shown in cartoon style, overlaid with the B-ribbon domain from the 1PIC structure as solvent accessible surface. The relocation is indicated by a magenta arrow. The antiparallel (3-sheet of Rap94 established with the clamp head in the 1ITC is marked by a magenta box.

### DETAILED DESCRIPTION

After reading this description, it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Specific”, “specifically”, “specificity”, or the like of a compound refers to the compound's ability to cause a particular action, such as inhibition, to a particular molecular target with minimal or no action to other proteins in the cell. In embodiments, a compound as described herein specifically reduces or inhibits activity of a viral polymerase, and/or specifically reduces or prevents interaction of a viral polymerase with one or more subunits or other factors.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its sequence reference, e.g., NCBI sequence reference. In other embodiments, the protein is the protein as identified by its sequence reference, homolog or functional fragment thereof.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell.

An “inhibitor” refers to a compound (e.g. compounds described herein) that reduces activity when compared to a control, such as absence of the compound or a compound with known inactivity.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity, or interaction, of a given gene or protein(s). The antagonist can decrease expression, activity, or interaction 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments, contacting includes allowing a compound described herein to interact with a protein or enzyme that is involved in a signaling pathway.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

### Methods

The instant technology generally relates to methods and compounds for regulating activity of a poxvirus viral polymerase in a cell infected with the poxvirus. In some aspects, regulating the activity of the poxvirus viral polymerase reduces or inhibits transcription of a viral gene(s) by the polymerase.

Without being bound by theory, it is believed that activity of a poxvirus viral polymerase can be modulated by modulating the interaction of one or more subunits of the polymerase with other subunits and/or the polymerase complex. For example, preventing formation of the complete polymerase complex may reduce transcription, e.g. by reducing (or preventing) the efficiency and/or initiation of transcription. In contrast, increasing interactions between one or more subunits may increase efficiency and/or initiation of transcription by the polymerase.

Further, and without being bound by theory, it is believed that modulation of the interaction of one or more subunits of the polymerase with other subunits and/or the polymerase complex may allow targeting of a poxvirus poxviral polymerase without affecting the activity of a host polymerase. For example, the compound may target a subunit that does not have a homologue in the host (subject or cell). Alternatively, the compound may target a subunit that is not normally associated with the host polymerase. Appendix A and Appendix B, submitted herewith and incorporated herein by reference in their entireties, describe viral RNA polymerase subunits that do not have homology to, or have low homology with, RNA polymerase subunits in S. cerevisae (e.g., Rap94), as well as subunits that interact with the viral RNA polymerase but are not known to act with RNA polymerase in other species, in particular eukaryotes (e.g., tRNA^{Glu}).

As used herein, the term “polymerase subunit” refers to any polypeptide/protein that associates with a polymerase. Polymerase subunits include, without limitation, subunits of the core polymerase, associated factors (transcription factors, capping enzymes, termination factors, chromatin remodeling enzymes, mRNA processing factors, elongation factors), and other viral transcription and RNA processing factors. See, Appendices A and B.

In an aspect, a method for regulating activity of a poxvirus viral polymerase in a cell infected with the poxvirus is provided. In embodiments, the method includes contacting the cell with a compound that reduces or prevents interaction of the viral polymerase with a glutamine tRNA (tRNA^{Glu}).

In an aspect, a method for treating or preventing infection by poxvirus in a subject in need thereof is provided. In embodiments, the poxvirus includes (or encodes) a viral polymerase and the method includes administering to the subject a compound that reduces or prevents interaction of the viral polymerase with a glutamine tRNA (tRNAGlu).

In an aspect, a method for modulating activity of a poxvirus viral polymerase in a cell infected with the poxvirus is provided. In embodiments, the method includes contacting the cell with glutamine. In embodiments, the glutamine modulates interaction of the viral polymerase with a glutamine tRNA (tRNA^{Glu}). In embodiments, the glutamine may reduce or prevent interaction of the viral polymerase with the tRNA^{Glu}. In embodiments, the glutamine may increase or promote interaction of the viral polymerase with the tRNA^{Glu}. In embodiments, the glutamine is a glutamine variant or glutamine analog.

In an aspect, a method for regulating activity of a poxvirus viral polymerase in a cell infected with the poxvirus is provided. In embodiments, the method includes contacting the cell with a compound that modulates activity of the viral polymerase. In embodiments, the compound reduces or inhibits activity of the viral polymerase. In embodiments, the compound enhances or promotes activity of the viral polymerase. In embodiments, the compound interacts with an active site of the viral polymerase.

In an aspect, a method for treating or preventing infection by poxvirus in a subject in need thereof is provided. In embodiments, the poxvirus includes (or encodes) a viral polymerase, and the method includes administering to the subject a compound that interacts with an active site of the viral polymerase.

In embodiments, the active site includes a binding site for a catalytic metal ion. In embodiments, the catalytic metal ion binding site is a D×D×D site on an Rpo147 subunit, or variant or homologue thereof. In embodiments, the compound reduces or inhibits binding of the catalytic metal ion to the binding site for the catalytic metal ion.

In embodiments, the compound reduces or inhibits interaction of subunit Rpo30 with the active site.

In embodiments, the compound interacts with an active site of a poxvirus capping enzyme. In embodiments, the compound reduces or inhibits activity of the poxvirus capping enzyme.

In embodiments, the compound inhibits or reduces interaction of one or more subunits of the viral polymerase from interacting with the viral polymerase. In embodiments, the one or more subunits of the viral polymerase include: Rpo147, Rpo132, Rpo35, Rpo22, Rpo19, Rpo18, Rpo7, Rpo30, Rap94, a capping enzyme, a termination factor, VETF-1, VETF-s, E11L, tRNA^{Glu}, NPH-1, VTF/CE, and/or any poxvirus polymerase subunit as listed or described in Appendix A and/or Appendix B, and/or a variant or homologue thereof. In embodiments, the one or more subunits of the viral polymerase include Rpo147 or a variant or homologue thereof. In embodiments, the one or more subunits of the viral polymerase include Rpo132 or a variant or homologue thereof. In embodiments, the one or more subunits of the viral polymerase include Rpo35 or a variant or homologue thereof. In embodiments, the one or more subunits of the viral polymerase include Rpo22 or a variant or homologue thereof. In embodiments, the one or more subunits of the viral polymerase include Rpo19 or a variant or homologue thereof. In embodiments, the one or more subunits of the viral polymerase include Rpo18 or a variant or homologue thereof. In embodiments, the one or more subunits of the viral polymerase include Rpo7 or a variant or homologue thereof. In embodiments, the one or more subunits of the viral polymerase include Rpo30 or a variant or homologue thereof. In embodiments, the one or more subunits of the viral polymerase include Rap94 or a variant or homologue thereof. In embodiments, the one or more subunits of the viral polymerase include a capping enzyme. In embodiments, the one or more subunits of the viral polymerase include a termination factor. In embodiments, the one or more subunits of the viral polymerase include VETF or a variant or homologue thereof. In embodiments, the one or more subunits of the viral polymerase include VETF-1 or a variant or homologue thereof. In embodiments, the one or more subunits of the viral polymerase include VETF-s or a variant or homologue thereof. In embodiments, the one or more subunits of the viral polymerase include E11L or a variant or homologue thereof. In embodiments, the one or more subunits of the viral polymerase include tRNA^{Glu} or a variant or homologue thereof. In embodiments, the one or more subunits of the viral polymerase include NPH-1 or a variant or homologue thereof. In embodiments, the one or more subunits of the viral polymerase include VTF/CE or a variant or homologue thereof.

In embodiments, the poxvirus is a variola virus or variant thereof. A variant of the variola virus may be, for example, an engineered or otherwise manipulated virus. For example, the variola virus may have been produces, engineered, and/or manipulated as a bioterrorism agent.

In embodiments, the poxvirus is a vaccinia virus or variant thereof. A variant of the vaccinia virus may be, for example, an engineered or otherwise manipulated virus. In embodiments, the vaccinia virus or variant thereof is a smallpox vaccine. In embodiments, the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains. In embodiments, the vaccinia virus is ACAM1000. In embodiments, the vaccinia virus is ACAM2000. In embodiments, the vaccinia virus is a New York City Board of Health strain. In embodiments, the poxvirus is an attenuated virus.

In embodiments, the viral polymerase is a virus-encoded RNA polymerase. In embodiments, the viral polymerase is a virus-encoded multisubunit RNA polymerase (vRNAP).

In embodiments, the compound is or includes a small molecule, an antisense RNA, a nucleic acid, an antibody, an aptamer, or a polypeptide. The compound may be any compound that interacts with the polymerase, such as a subunit, active site, or other component of the polymerase. The compound may inhibit binding of a subunit, active site, or other component of the polymerase to other components of the polymerase, thereby preventing formation of a complete polymerase complex.

Antibodies to various subunits of poxvirus RNA polymerase are known. See, e.g, Satheshkumar et al., J Virol. 2013 October; 87(19): 10710-10720, which is incorporated herein by reference in its entirety. Similarly, compounds that bind tRNAs are known. See, e.g., Connelly et al., Cell Chemical Biology (2016) 23:1077-1090; U.S. Patent App. Pub. 2003/0008808; each of which is incorporated herein by reference in its entirety.

In embodiments, the infected cell is a stem cell, immune cell, or cancer cell. In embodiments, the stem cell may be an adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, or any combination thereof.

The compound may be any compound having the described activity. Methods for identifying small molecule compounds that will interact with a target are described, for example, in Kubinyi, H. (2006), ‘Success Stories of Computer-Aided Design’, in Ekins, S. (ed.) Computer Applications in Pharmaceutical Research and Development. John Wiley & Sons, Inc., pp. 377-417, which is incorporated herein by reference in its entirety.

Compounds that may have an effect on viral RNA polymerase activity include, without limitation, the following compounds, including variants thereof:

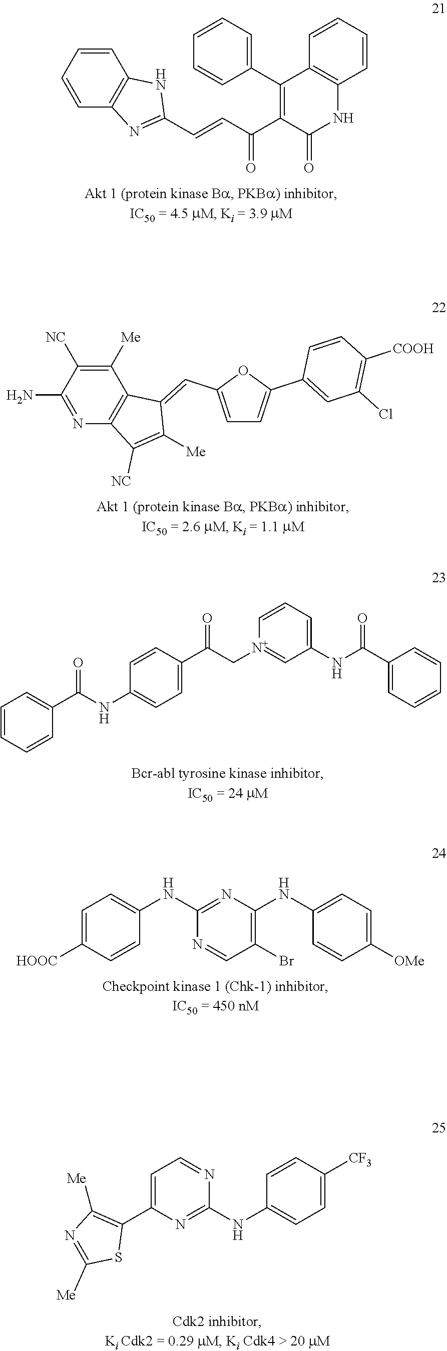 [SMILES: O=C(/C=C/c1nc2ccccc2[nH]1)c1c(-c2ccccc2)c2ccccc2[nH]c1=O ; CC1=C(C#N)c2nc(N)c(C#N)c(C)c2/C1=C/c1ccc(-c2ccc(C(=O)O)c(Cl)c2)o1 ; O=C(C[n+]1cccc(NC(=O)c2ccccc2)c1)c1ccc(NC(=O)c2ccccc2)cc1 ; COc1ccc(Nc2nc(Nc3ccc(C(=O)O)cc3)ncc2Br)cc1 ; Cc1nc(C)c(-c2ccnc(Nc3ccc(C(F)(F)F)cc3)n2)s1]

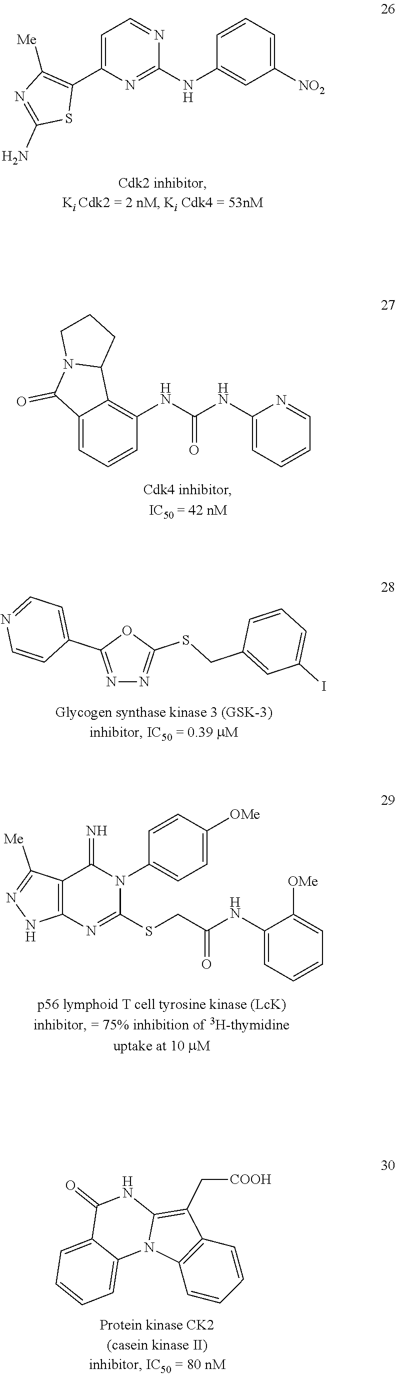 [SMILES: Cc1nc(N)sc1-c1ccnc(Nc2cccc([N+](=O)[O-])c2)n1 ; O=C(Nc1ccccn1)Nc1cccc2c1C1CCCN1C2=O ; Ic1cccc(CSc2nnc(-c3ccncc3)o2)c1 ; COc1ccc(-n2c(SCC(=O)Nc3ccccc3OC)nc3[nH]nc(C)c3c2=N)cc1 ; O=C(O)Cc1c2ccccc2n2c1[nH]c(=O)c1ccccc12]

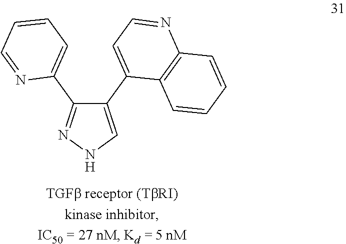 [SMILES: c1ccc(-c2n[nH]cc2-c2ccnc3ccccc23)nc1]

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that descriptions of making and using the particles described herein is for the sole purpose of illustration, and that the present disclosure is not limited by this illustration.

Appendix A and Appendix B are submitted herewith, and are incorporated herein by reference in their entireties.

### Example 1. Glutamine is Required for Later Virus Production, but not for Initial Infection of CV-1 Cells

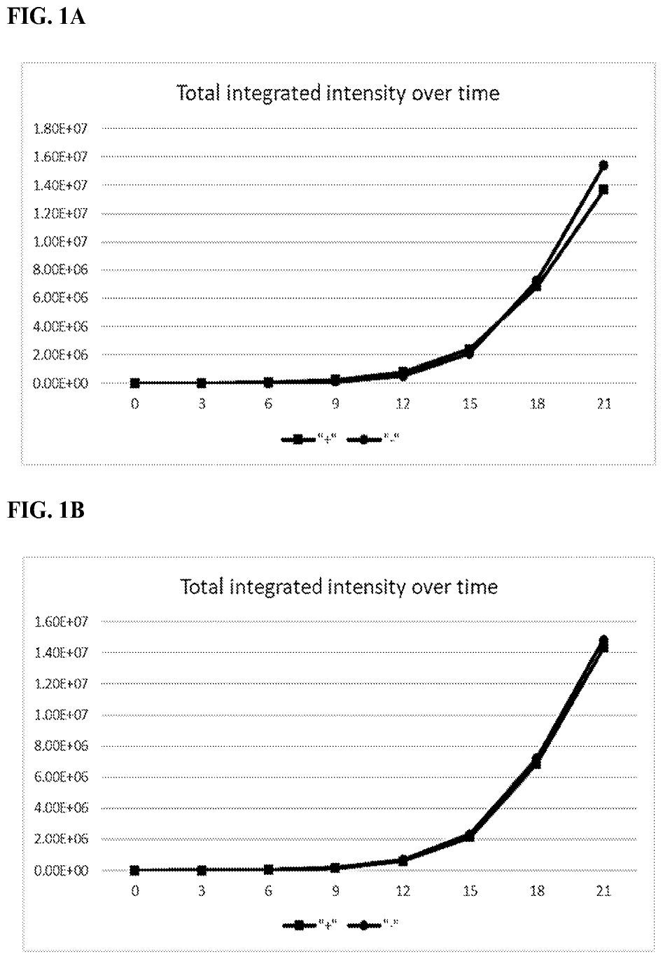

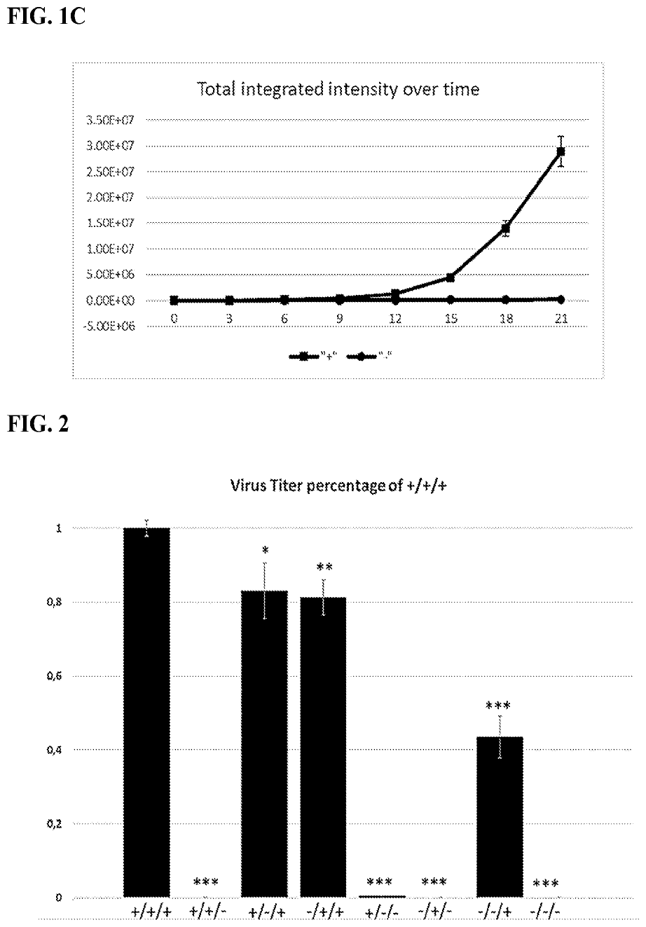

FIGS. 1A through 1C depict a clear trend, that glutamine absence during the third medium switch has a severe impact on intensity. Namely, samples without glutamine in the third switch show an intensity roughly 100 times lower than their counterparts. In FIGS. 1A and 1B, no real distinction between glutamine presence/absence can be made. This displays the first indication of the negligence of glutamine in the first two medium switches. In contrast, FIG. 1C shows a contrary correlation. There glutamine absence resulted in a final intensity (after 21 hours) with a value over 100 times lower than in glutamine fed samples. Since infection in glutamine absence still took place and the two graphs only part their way after six hours, it can be postulated that the different intensity is not rooted in altered virus permissiveness to the infected cells. Instead, it appears that glutamine absence somehow decreases virus replication drastically.

A virus production assay (VPA) was performed to confirm this finding. The VPA allows a numerical evaluation of the virus titer during glutamine depletion. Since multiple rounds of infection are prevented by adding CMC, differences are not exponentiated, thus allowing a reliable comparison of the samples.

The most striking observation from FIG. 2 is the drastic decrease of the virus titer in samples without glutamine in the third medium switch. The titer percentage of these samples ranges between 0.08% and 0.06%. This means that samples with glutamine in the third medium switch show a virus replication more than 1000 times higher than their negative counterparts (Table 1).

TABLE 1
Virus titer

| Negative | Virus Titer | Positive | Virus Titer | Factor |
|---|---|---|---|---|
| +/+/− | 2.73E+03 | +/+/+ | 3.34E+06 | 1222 |
| +/−/− | 1.89E+03 | +/−/+ | 2.78E+06 | 1468 |
| −/+/− | 1.53E+03 | −/+/+ | 2.72E+06 | 1774 |
| −/−/− | 7.27E+02 | −/−/+ | 1.45E+06 | 2000 |

Interestingly, an increase in titer is observed even when glutamine was absent in only the first and/or second media changes. While some residual glutamine may remain in the wells and/or cell cytosol in the glutamine-negative conditions, this cannot fully account for these findings. Thus, it is likely that glutamine improved virus replication even in the first and second medium switches. This thesis is furthermore supported by the “−/−/−” and the “−/−/+” samples, which show the lowest virus titers, while not being supplemented with glutamine during the first and second medium switch. From this, it follows that glutamine apparently influences VACV replication during entry into the cells or even before. Glutamine requirement during the first hour of infection would mean that glutamine somehow supports vaccinia prior to the start of replication.

Methods

Cell Culturing

CV-1 cells were cultured in 25 mL of DMEM GlutaMAX supplemented with 10% FBS. Once confluency of ˜90% was observed under the microscope, adherent cells were passaged or harvested via trypsinization. To ensure that no cells were still attached to the cell surface, the supernatant was repeatedly applied to the flask surface by a pipette with force. Prior to trypsinization cells were washed with PBS two times to remove FBS remains, which would interfere with the activity of trypsin.

Glutamine Experiment

Via trypsinization, harvested cells were centrifuged at 4000 RPM at 23° C. for five minutes. The supernatant was carefully removed with the vacuum pipette and the cell pellet was resuspended in MEM medium supplemented with 10% dialyzed FBS and 2.5% L-Glutamine solution. 10 μl of each cell solution and trypan blue were mixed in a 1 mL Eppendorf tube, plotted on a cell counting plate and the cell number was determined by the cell counter. Through the measured cell count, the volume that contains 2.5×10⁶ cells was calculated and extracted. Said volume was diluted to 25 mL with the prepared MEM. Those cells were then seeded in a 24 well plate at a density of 1×10⁵ cells/well/mL. Approximately four hours after seeding, when cells were already attached to the surface of the well, the medium was removed with the vacuum pipette. Fresh MEM medium with 10% dialyzed FBS was added, with or without the addition of 5 mM L-glutamine for each medium switch. A second medium switch was performed at the point of infection, and a third at one hour post-infection.

At the second medium switch, the point of infection, cells were infected with Clopt1 (Vaccinia virus) at an MOI of 2 in 200 μL of infection medium (MEM medium supplemented with 2% dialyzed FBS and 5 mM L-glutamine if required). In the third medium switch, one hour post-infection, 1 mL of infectious medium was added to each well and the plates were scanned in the IncuCyte every three hours for a period of 21 hours. After the scans were completed, cells and their supernatant were transferred to a 1 mL Eppendorf tube. Cells were once again harvested via trypsinization after being washed with PBS two times. The tubes were then stored at −80° C. until further use. An analysis of the scans was created with the integrated tool of the IncuCyte software.

Virus Production Assay

Stored cells were frozen in liquid nitrogen, then thawed in a 37° C. warm water bath and vortexed for 30 seconds after. This process was repeated three times to achieve a complete disassociation of cells and virus particles. For each sample, a serial dilution from 10⁻¹ to 10⁻⁶ was prepared in a 48 well plate. From each sample, 60 μL were added to 540 of DMEM GlutaMAX supplemented with 2% FBS. 250 μL of each well were used to infect confluent CV-1 cells in 24 well plates DMEM GlutaMAX medium supplemented with 10% FBS. Those cells were seeded on the previous day at a density of 8×10⁴ cells/well/mL in DMEM GlutaMAX with 10% FBS. One hour post-infection 1 mL of CMC was added to each well as an overlay medium. 48 hours post-infection roughly 800 μL of the medium was removed and 200-300 μL of crystal violet was added to each well. Plates were then placed on a shaker overnight. On the next day, after the supernatant was removed, plates left to dry for several days. To determine the plaque count, dry well plates were placed on a light pad. The visible plaques from one dilution of each sample were then counted by eye. If possible, wells with approximately 15-100 PFUs were chosen for counting.

### Example 2. Structural Basis of Poxvirus Transcription: Transcribing and Capping Vaccinia Complexes

Poxviruses use virus-encoded multi-subunit RNA polymerases (vRNAP) and RNA-processing factors to generate m⁷G-capped mRNAs in the host cell cytoplasm. In the accompanying Examples are reported structures of core and complete vRNAP complexes of the prototypic Vaccinia poxvirus (Grimm et al., Example 3). Here is presented the cryo-EM structures of Vaccinia vRNAP in form of a transcribing elongation complex and in form of a co-transcriptional capping complex that contains the viral capping enzyme. The trifunctional capping enzyme forms two mobile modules that bind to the polymerase surface around the RNA exit tunnel. RNA extends from the vRNAP active site through the exit tunnel and into the active site of the capping enzyme triphosphatase. Structural comparisons suggest that growing RNA triggers large-scale rearrangements on the surface of the viral transcription machinery during the transition from transcription initiation to RNA capping and elongation. These structures reveal the basis for synthesis and co-transcriptional modification of poxvirus RNA.

Poxviruses belong to a group of DNA viruses with exceptionally large genomes that replicate in the host cytoplasm. Vaccinia, the non-pathogenic virus strain used as a smallpox vaccine and as promising agent in oncolytic virotherapy, contains a ˜190 kbp double-stranded DNA genome that is transcribed in the cytosol by an eight-subunit virus-encoded RNA polymerase (vRNAP) (Broyles, 2003; Frentzen et al.). While most of these subunits share sequence homology to subunits of cellular RNA polymerase II (Pol II), their degree of similarity differs from very strong to barely detectable (Ahn et al., 1990; 1992; Amegadzie et al., 1992; 1991; Broyles and Moss, 1986; Knutson and Broyles, 2008; Mirzakhanyan and Gershon, 2017; Patel and Pickup, 1989). In addition to the core vRNAP enzyme, Vaccinia employs numerous virus-specific transcription factors, most of which appear evolutionarily unrelated to host transcription factors (Mirzakhanyan and Gershon, 2017). This includes factors required for transcription initiation, elongation and termination (Broyles, 2003).

Poxviral transcripts bear a 5′-cap and a poly-A tail, and thus resemble mRNAs generated by the host cell. The cap structure consists of an N⁷-methylated guanosine residue linked to the 5′-end of the nascent transcript via an inverted 5′-5′ triphosphate linkage (Ghosh and Lima, 2010). Capping occurs co-transcriptionally shortly after transcription initiation by the sequential action of three enzymes (Moteki and Price, 2002): First, a triphosphastase (TPase) hydrolyses the 5′-triphosphate of the RNA to yield a 5′-diphosphate. A guanlyltransferase (GTase) then catalyzes the addition of guanosine monophosphate (GMP), which is subsequently methylated by the action of a methyltransferase (MTase). The three capping enzyme activities can be encoded by three separate enzymes, as found in fungi, or by multi-functional proteins. While metazoans utilize a difunctional TPase-GTase polypeptide in which the TPase is evolutionarily unrelated to those found in fungi, many viruses use trifunctional enzymes (Ghosh and Lima, 2010).

The poxviral capping enzyme (CE) is a heterodimer of the D1 and D12 subunits. D1 is a trifunctional enzyme that harbors all three enzymatic activities required for cap synthesis (Cong and Shuman, 1992; Martin and Moss, 1975; Shuman and Morham, 1990). D12 binds to the MTase domain of D1 and stimulates its activity allosterically, as shown by previous biochemical and crystallographic studies of the enzyme (Kyrieleis et al., 2014; Mao and Shuman, 1994). Structural information on yeast, mammalian and poxviral CEs has been reported, but it is unclear how these enzymes interact with RNA substrates (Fabrega et al., 2004; Ghosh et al., 2011; Gu et al., 2010; la Peña et al., 2007). A cryo-EM reconstruction of the S. cerevisiae Pol II-CE complex showed that CE docks to the body of transcribing Pol II, but mechanistic insights could not be obtained due to the low resolution (Martinez-Rucobo et al., 2015).

Viral gene expression typically follows defined temporal patterns, which are referred to as early, intermediate and late transcription. Early genes are activated shortly following infection and encode proteins necessary for the expression and replication of the viral genome. In poxviruses, specific transcription factors facilitate early gene transcription. Initiation is mediated by Rap94 and the very early transcription factor (VETF) (Ahn et al., 1994; Broyles et al., 1991; 1988; Cassetti and Moss, 1996). Following initial transcription, capping occurs when the nascent RNA reaches a length of 27-31 nucleotides (nt) (Hagler and Shuman, 1992a). The CE is not only required for capping but also during termination of early gene transcription and is therefore also referred to as Vaccinia Termination Factor (VTF) (Luo et al., 1995). Termination is mediated by a signal sequence in the nascent RNA and requires, in addition to CE, the helicase Nucleoside Triphosphatase I (NPH-I) (Christen et al., 1998; Rohrmann et al., 1986; Shuman et al., 1987).

In the accompanying Examples, the purification and structural analysis of viral transcription complexes from human cells infected with a recombinant Vaccinia virus strain (Example 3) are described. These studies revealed the structure of the eight-subunit core vRNAP enzyme as well as of the complete vRNAP complex with early viral transcription factors. The latter contains, in addition to the core vRNAP enzyme, the transcription factors Rap94, VETF, CE, NPH-I, the structural protein E11 and host tRNA^{Gln}. This complex is capable of early promoter-dependent transcription initiation, elongation and termination. It thus represents the unit facilitating early gene transcription that may also be packaged into viral progenies.

These structures uncovered the architecture of vRNAP and its interaction with transcription factors. However, how the vRNAP machinery interacts with nucleic acids to achieve transcription and RNA modification remained unknown. Here, is determined the structures of actively transcribing vRNAP complexes. The structure of vRNAP bound to a DNA template and an RNA transcript reveals a similar mechanism of transcript elongation as shown for other multisubunit RNA polymerases. The structure of transcribing vRNAP bound to CE illustrates the path of the RNA from the active site of the polymerase to one of the active sites of the capping enzyme and unravels structural rearrangements that occur during the transition from transcription initiation to elongation. Together, these results provide a framework for future mechanistic analysis of the transcription cycle of viral multi-subunit RNA polymerases.

Preparation of vRNAP Transcribing Complexes

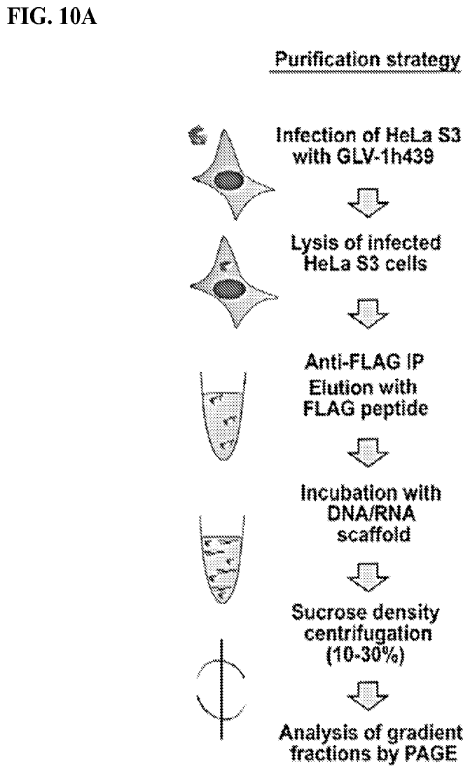

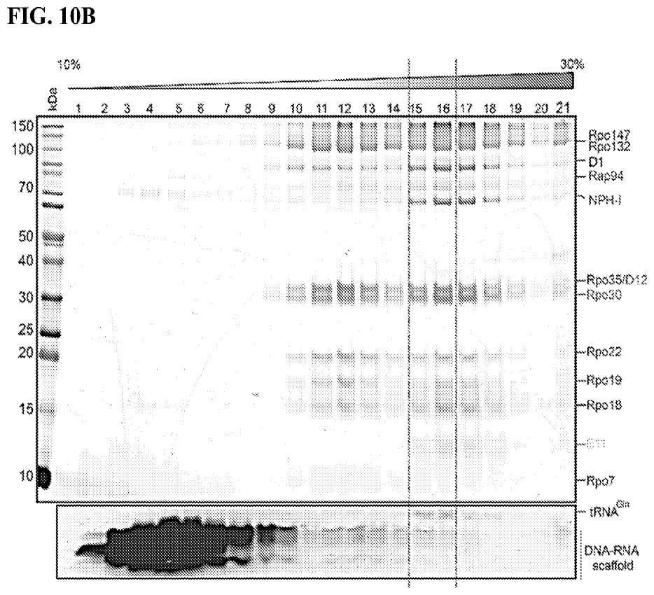

Vaccinia vRNAP complexes were purified as described (Example 3) and transcribing complexes were formed on a DNA/RNA scaffold consisting of a double-stranded DNA with a mismatch bubble (FIG. 10A), a strategy previously used for the structural characterization of Pol I, II and III (Hoffmann et al., 2015; Kettenberger et al., 2004; Neyer et al., 2016). The DNA fragment was derived from an early gene in the Vaccinia genome, encoding the largest subunit of vRNAP, Rpo147. To mimic the nucleic acids in an actively transcribing complex, the single-stranded template strand in the mismatched region was hybridized to RNA that contained nine nucleotides at its 3′ end that were complementary to the template strand.

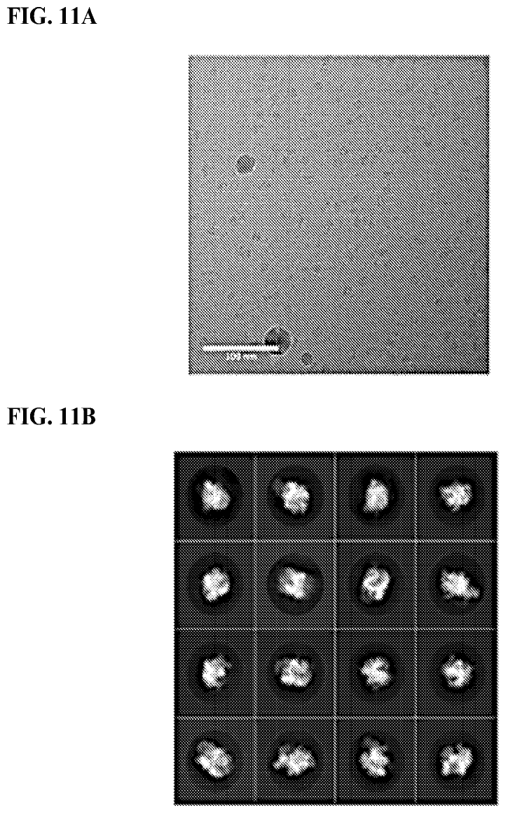

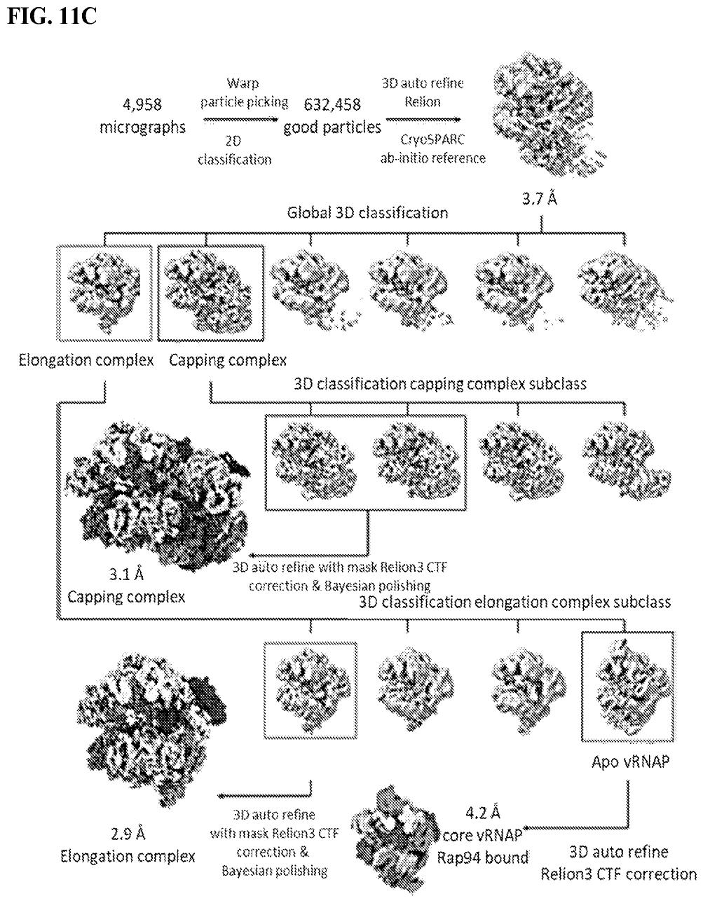

To facilitate stabilization of a co-transcriptionally capping complex, the RNA was produced by in vitro transcription in order to contain the 5′-triphosphate moiety also found in naturally synthesized transcripts. A 31 nt RNA was chosen based on previous results demonstrating that co-transcriptional capping occurs at a nascent RNA length of 27-31 nt (Hagler and Shuman, 1992a). To assemble vRNAP elongation complexes, vRNAP was incubated with a large excess of the pre-formed DNA/RNA scaffold following initial FLAG-purification (FIG. 11A). After further purification by sucrose gradient centrifugation, two populations with distinct sedimentation coefficients could be observed, similar to the previously observed vRNAP complexes lacking nucleic acids (FIG. 11B).

Structure Determination of vRNAP Bound to Nucleic Acids

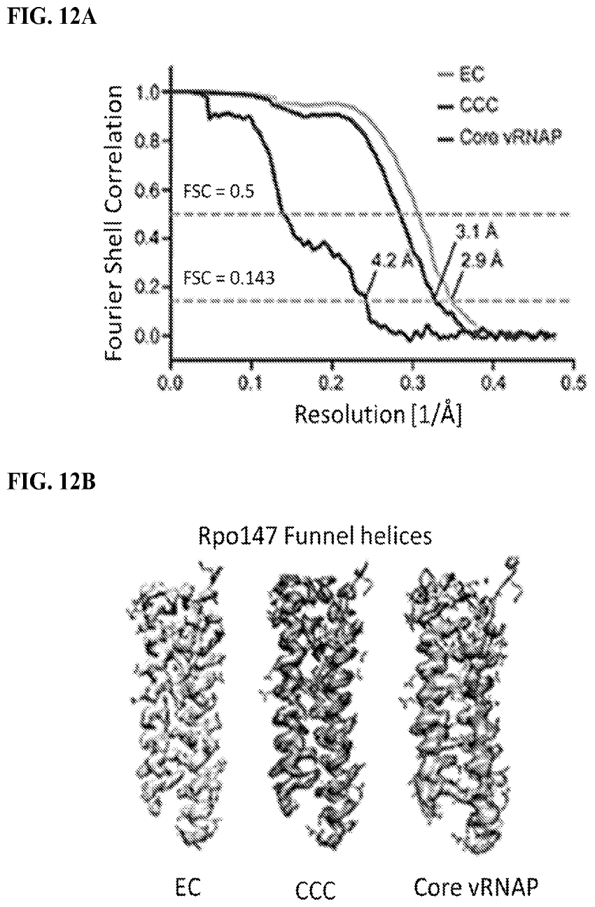

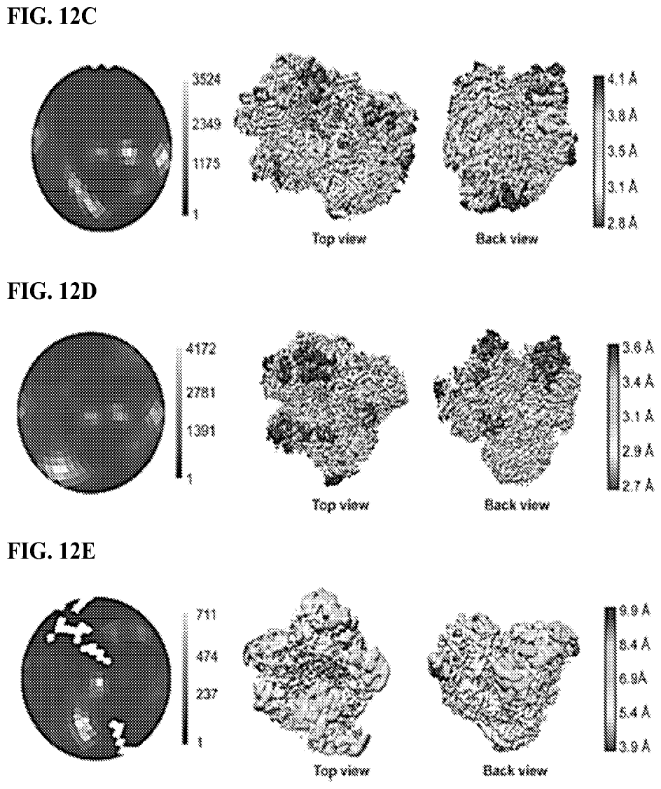

The fractions corresponding to the larger molecular weight complex were subjected to single-particle cryo-EM analysis. Unsupervised 3D classification of the obtained dataset revealed two distinct populations of particles (FIG. S2). The first resembled closely the previously determined core vRNAP structure (Grimm et al., submitted in parallel), but showed additional density for nucleic acids in the active center cleft. The second class showed large additional densities on the enzyme surface where the nascent RNA is expected to emerge. Further sub-classification and 3D refinement yielded high-resolution reconstructions for both classes at 3.0 Å and 3.2 Å, respectively (FIGS. 11 and 12).

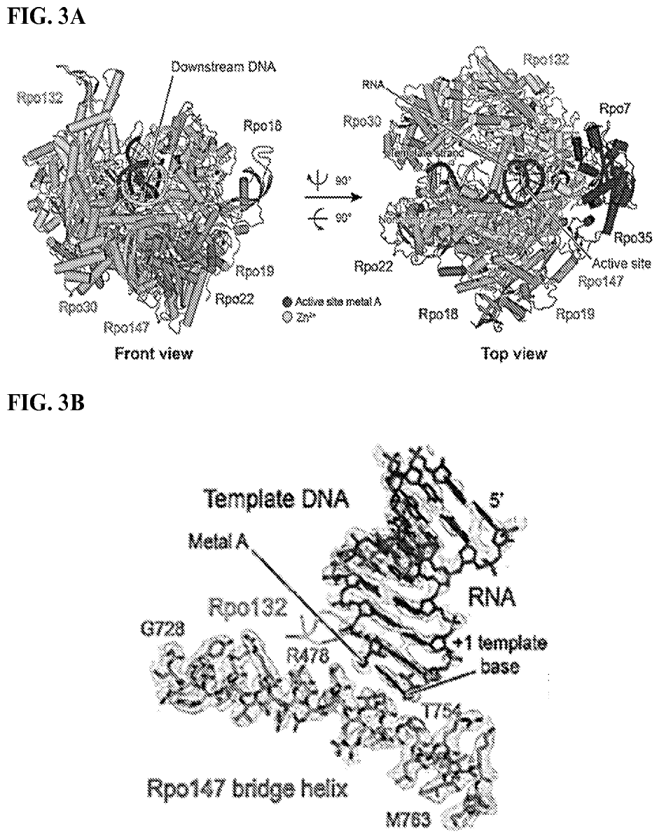

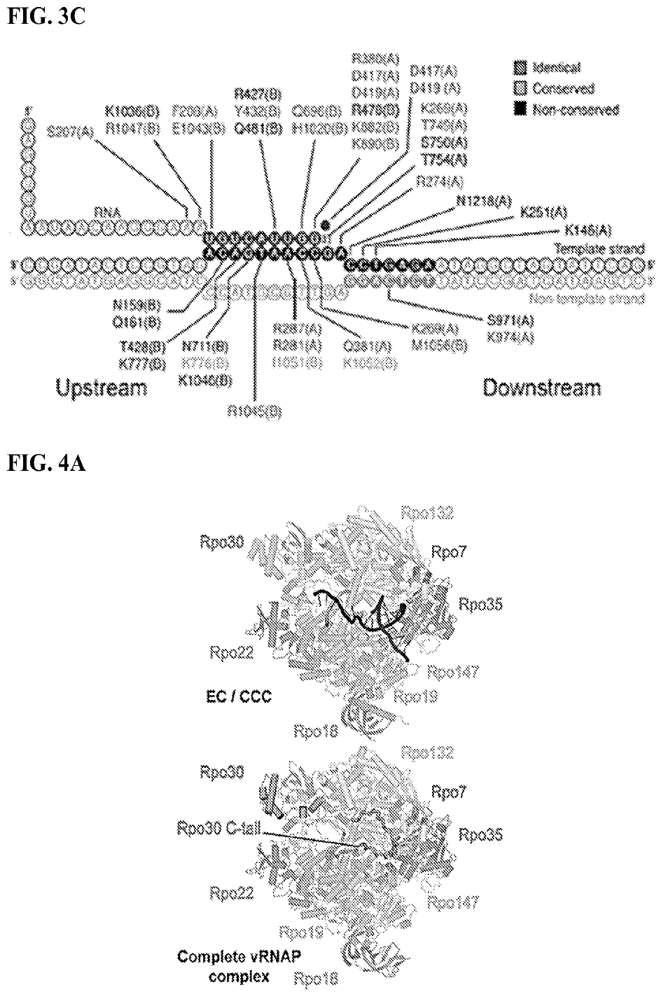

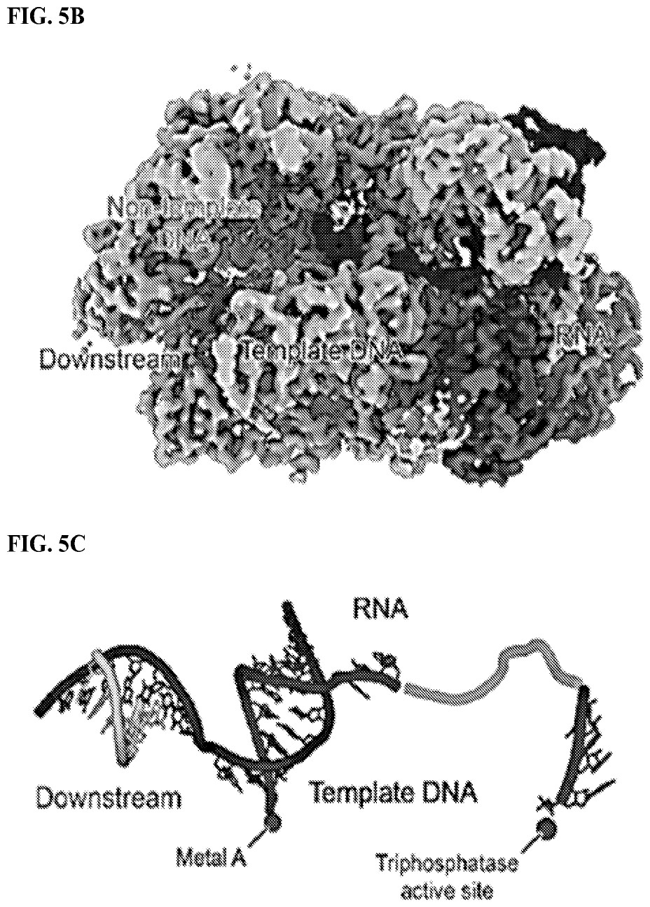

Analysis of the obtained densities confirmed that the first complex represents an elongation complex (EC) consisting of the core vRNAP enzyme with nucleic acids in the active center cleft (FIG. 3A). The density for the nucleic acid was of high quality around the DNA-RNA hybrid (FIG. 3B), and somewhat weaker for downstream DNA. Density for the single-stranded portion of the non-template DNA strand and for the upstream DNA became visible at low thresholds but did not allow for modelling (FIG. 5C). The large additional density in the second reconstruction could be fitted with the crystal structure of Vaccinia CE (Kyrieleis et al., 2014). Continuous RNA density was observed stretching from the vRNAP active site to the active site of the CE TPase (FIG. 5B). Thus, the second reconstruction represents a co-transcriptional capping complex (CCC).

Structure of the vRNAP Elongation Complex

The structure of the vRNAP EC reveals the active state of the enzyme. The overall structure of the eight-subunit polymerase is largely unchanged compared to the core vRNAP structure described in the accompanying Example (FIG. 3A). However, the viral transcription factor Rap94, which was associated with both core and complete vRNAP structures, is absent from the EC structure. The active center cleft is occupied by a 9 basepair (bp) long DNA-RNA hybrid (FIG. 3B). This is reminiscent of other multisubunit and single subunit RNA polymerases, which all bind an 8-9 bp hybrid in their active center (Cramer, 2002; Martinez-Rucobo and Cramer, 2012).

In the structure, vRNAP adopts the active, post-translocated state (FIG. 3B). The binding site for the nucleoside triphosphate substrate is empty and the +1 template base is positioned for base pairing along the bridge helix that spans the polymerase cleft. The duplex axes of downstream DNA and the hybrid enclose an angle of ˜90°. Analysis of the protein-nucleic acid interactions in the vRNAP EC reveals a high structural conservation to eukaryotic cellular RNA polymerases. The majority of residues involved in nucleic acid interactions are either identical or conserved in S. cerevisiae Pol II (FIG. 3C).

There are also some notable differences in the active sites of vRNAP as compared to cellular RNA polymerases. In particular, residue T754 in the bridge helix binds to the template DNA strand in between bases at positions +1 and +2. The corresponding residue is strictly conserved as tyrosine in Pol I, II and III (Y836 in S. cerevisiae Pol II) (Gnatt et al., 2001). In addition, residue R478 in Rpo132 is unique to vRNAP, as this position is strictly conserved to glycine in cellular polymerases. In vRNAP the arginine side chain projects towards the terminal 3′ nucleotide of the RNA and to the binding site for the substrate nucleoside triphosphate, and may be involved in early RNA synthesis. The conformation of the trigger loop, a structural element involved in catalysis by multisubunit RNA polymerases (Martinez-Rucobo and Cramer, 2012), appears most similar to the ‘locked’ conformation in the Pol II-TFIIS reactivated complex (Cheung and Cramer, 2011). Despite these differences, these results indicate that the fundamental mechanism of DNA-dependent RNA synthesis is conserved between cellular and viral multisubunit RNA polymerases.

Release of the Rpo30 Tail from the Catalytic Center

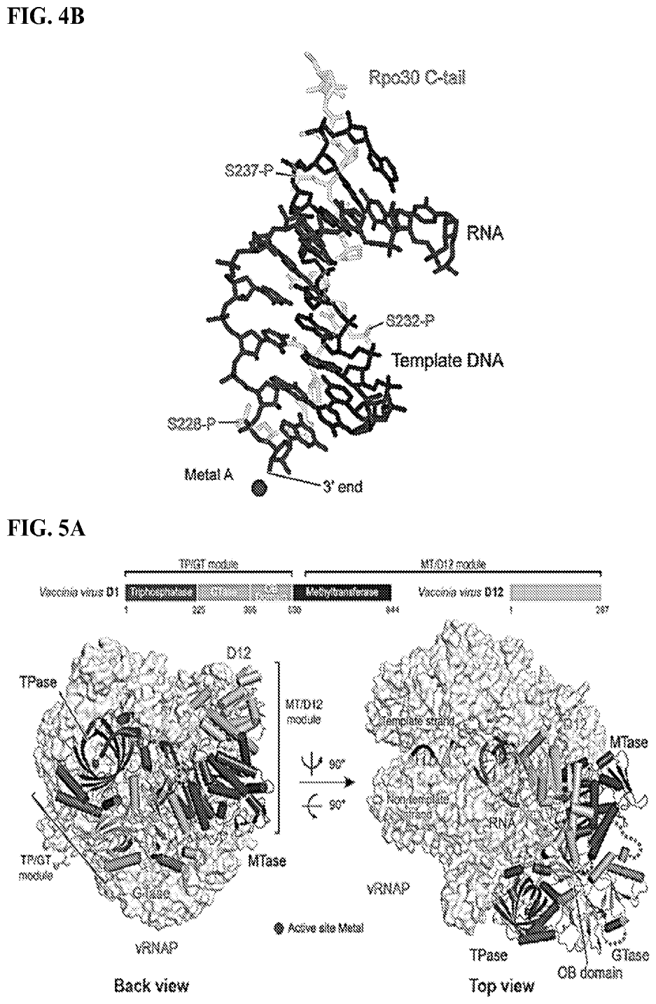

The EC structure also suggest rearrangements that must occur during the transition from the complete vRNAP structure to the EC. The complete vRNAP structure revealed a surprising feature of the vRNAP subunit Rpo30. This subunit showed a phosphorylated C-terminal tail that binds the active center (Grimm et al., submitted in parallel). Comparison of the EC structure described here with the complete vRNAP complex demonstrates that the Rpo30 C-terminal tail would clash with both DNA and RNA in the hybrid duplex (FIG. 4A). In particular, the phosphate moieties on residues S228, S232 and S237 overlap with the positions of backbone phosphate groups in the hybrid (FIG. 4B). Whereas the phosphorylated residue S228 occupies the phosphate binding site of the most 3′ RNA nucleotide in the EC, the phosphorylated residues S232 and S237 bind to the phosphate positions occupied by nucleotides −3 and −7, respectively, in the template DNA strand. These results suggest that the Rpo30 tail can inhibit vRNAP in a phosphorylation-dependent manner. It is speculated that Rpo30 phosphorylation provides a mechanism to regulate viral gene expression during the cellular replication phase and/or during the transition from the packaged state to the actively transcribing state.

Structure of vRNAP Co-Transcriptional Capping Complex

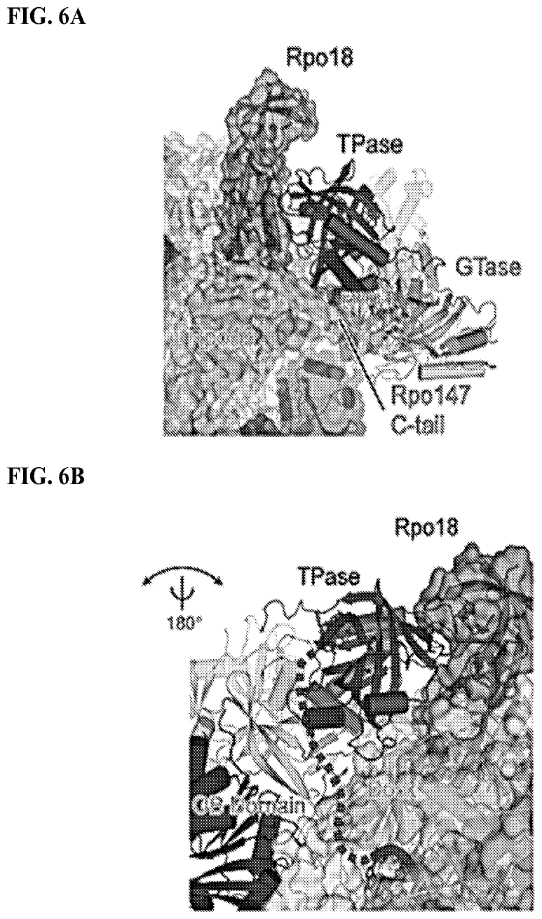

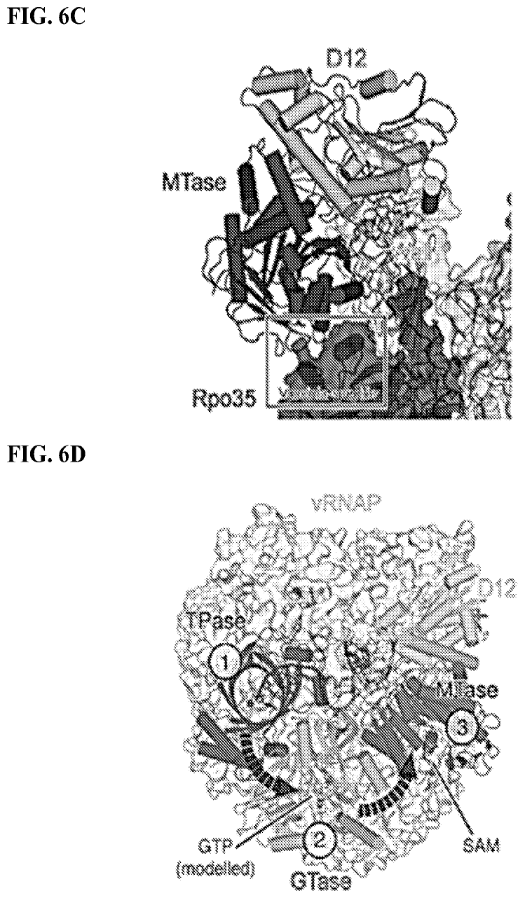

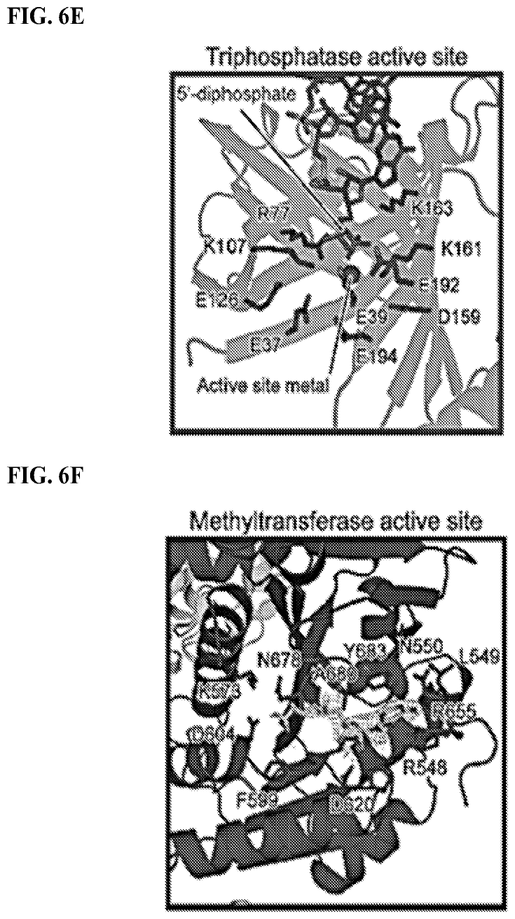

The structure of the CCC reveals the viral polymerase during co-transcriptional capping. The conformation of the polymerase is essentially identical to that observed in the EC structure. The viral capping enzyme is bound around the site where RNA exits the enzyme (FIG. 5A). Both subunits of CE, D1 and D12, engage in interactions with vRNAP, mainly to subunits Rpo147, Rpo132, Rpo18 and Rpo35 (FIG. 5A and FIGS. 6B-6D). The DNA-RNA hybrid is observed in the active center cleft, but the structure also reveals the trajectory of the RNA beyond the hybrid (FIG. 5B). At the upstream edge of the hybrid, the conserved residue F208 in the lid loop of vRNAP subunit Rpo147 separates RNA from the DNA template strand. RNA density is continuous through the RNA exit tunnel of the enzyme and over the surface of CE until its 5′-end, for which four bases are seen in the active site of the TPase domain of D1 (FIGS. 5B and 5C, FIG. 6E). The RNA appears to be partially mobile and scrunched in a central region located between the end of the hybrid and the TPase active site (Methods). Taken together, the CCC structure uncovers the architecture of transcribing vRNAP during capping, and reveals the path of the nascent RNA from the vRNAP active site to the CE TPase active site.

Capping Enzyme Contains Two Mobile Modules

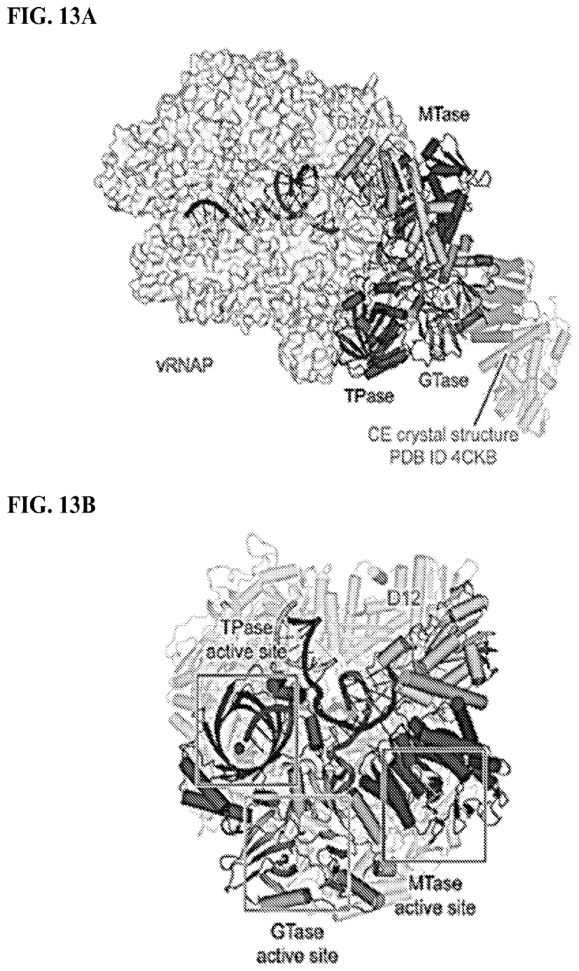

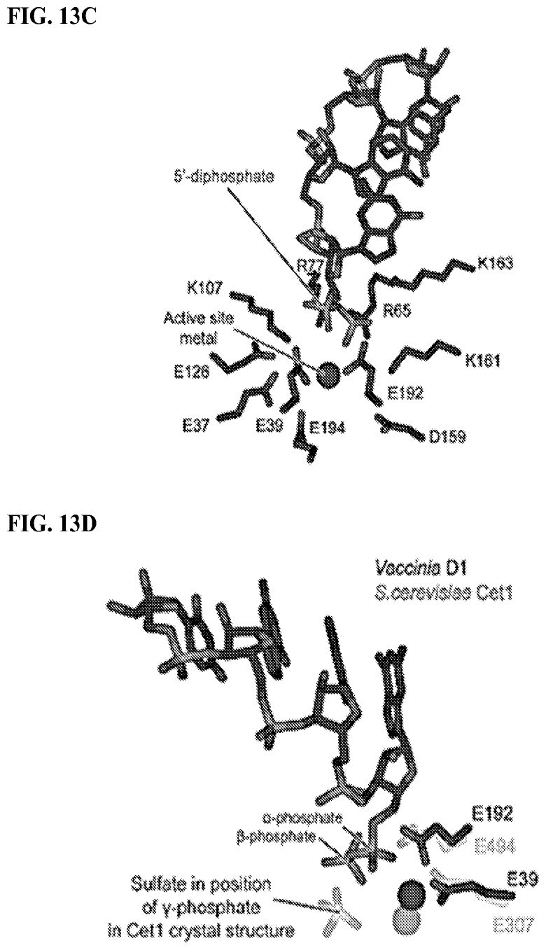

Superposition of polymerase-bound CE with the free CE crystal structure (Kyrieleis et al., 2014) reveals that the individual CE domains are essentially identical (FIG. 13A). However, the superposition also indicates that CE consists of two modules that can move with respect to each other. Whereas one module contains the TPase and GTase domains of subunit D1 (‘TP/GT module’), the other module consists of the MTase domain of D1 and subunit D12 (‘MT/D12 module’) (FIG. 5A). Relative movement of the two CE modules with respect to each other is enabled by a flexible intermodule linker (res. 529-560), as predicted (Kyrieleis et al., 2014). The observed conformation of CE positions the MT/D12 module in close proximity to the polymerase. Furthermore, the region between residues 116 and 124 of D12 is located close to exiting RNA, potentially enabling further interactions with the substrate. Thus, CE consists of two mobile modules that adopt a distinct relative orientation when CE binds to transcribing vRNAP. As a result, the three active sites of CE are positioned in the vicinity of the exiting RNA (FIG. 13B), likely facilitating the shuttling of RNA between active sites during subsequent reaction steps (FIG. 6A).

Interactions Between vRNAP and Capping Enzyme

The CCC structure reveals the detailed interactions between vRNAP and CE subunits D1 and D12 (FIGS. 6B-6D). The TPase domain stacks against the large subunit of vRNAP and against the stalk subunit Rpo18 (FIGS. 6B and 6C). As observed previously in the complete vRNAP complex (Grimm et al., submitted in parallel), the C-terminal tail (C-tail) of Rpo147 interacts with D1 by inserting its terminal residue F1286 into a pocket formed at the interface of the TPase and GTase domains (FIG. 6B). Further interactions are mediated by the Dock domain of vRNAP, which is sandwiched between the TPase domain and the OB fold of D1 (FIG. 6C). The latter two form a positively charged groove, along which the RNA is guided towards the TPase active site. In addition, Y409 in the OB fold may form stacking interactions with bases of the nascent RNA. The MTase domain and subunit D12 are positioned on the opposite side of the groove, where they bind to the Wall domain in Rpo132 (FIG. 6D). The MTase domain contacts region 164-171 of Rpo35, which is absent in the corresponding Pol II subunit Rpb3 (FIG. 6D). The MTase domain is connected to the OB fold by a flexible linker, which was also mobile in the previously reported crystal structure of the CE (Kyrieleis et al., 2014). Taken together, CE forms a set of viral-specific contacts with the polymerase around the site of RNA exit.

Interactions of the Triphosphatase with the 5′-End of RNA

The structure of the CCC also reveals the interactions between nascent RNA and CE during the first step of cap formation. The RNA 5′ end is stably bound to the TPase domain of CE (FIGS. 5A and 5C, FIGS. 6A and 6E). The TPase active site is located inside a beta-barrel structure, with basic residues lining one side and acidic residues lining the opposite side (FIG. 6E) (Kyrieleis et al., 2014). The structure uncovers that the RNA enters the barrel from the side previously proposed (Kyrieleis et al., 2014). The cryo-EM density observed within the active site, together with chemical considerations, is most consistent with a 5′-diphosphate moiety on the RNA, contacted by a catalytic metal ion (FIG. 6E and FIG. 13C). This is corroborated by a comparison to the structure of the S. cerevisae TPase homologue Cet1, which shows a very similar arrangement of basic and acidic residues in the barrel (Gu et al., 2010; Lima et al., 1999). Although lacking a substrate RNA, the Cet1 structure contains a catalytic metal ion and a sulfate ion that may mimic the leaving γ-phosphate. Superposition with the Cet1 structure positions this sulfate ion immediately adjacent to the 5′-diphosphate of the RNA in this structure, where the γ-phosphate is expected prior to cleavage (FIG. 13D). Thus, the CCC structure appears trapped after cleavage of the γ-phosphate and represents a product complex for the first step of co-transcriptional capping.

Guanylyltransferase and Methyltransferase

Following formation of the 5′-diphosphate, a GMP-moiety is added to the nascent RNA and this reaction proceeds via an enzyme-GMP intermediate in the GTase active site of D1 (Ghosh and Lima, 2010). GTP was omitted in the sample and therefore the GTase active site is empty (FIG. 13B). On the other hand, analysis of the MTase active site revealed density at the location where S-adenosyl-homocysteine (SAH) was observed in a SAH-bound crystal structure (Kyrieleis et al., 2014). The density is in good agreement with the S-adenosyl-methionine (SAM) cofactor required to methylate the RNA substrate (FIG. 6F). Since SAM, like GTP, was not added during purification and sample preparation, it likely originates from the source cells and was stably bound during the purification procedure. Understanding the structural mechanisms underlying the second and third steps of capping would require trapping of the CCC in corresponding functional states.

Capping Enzyme Rearrangements

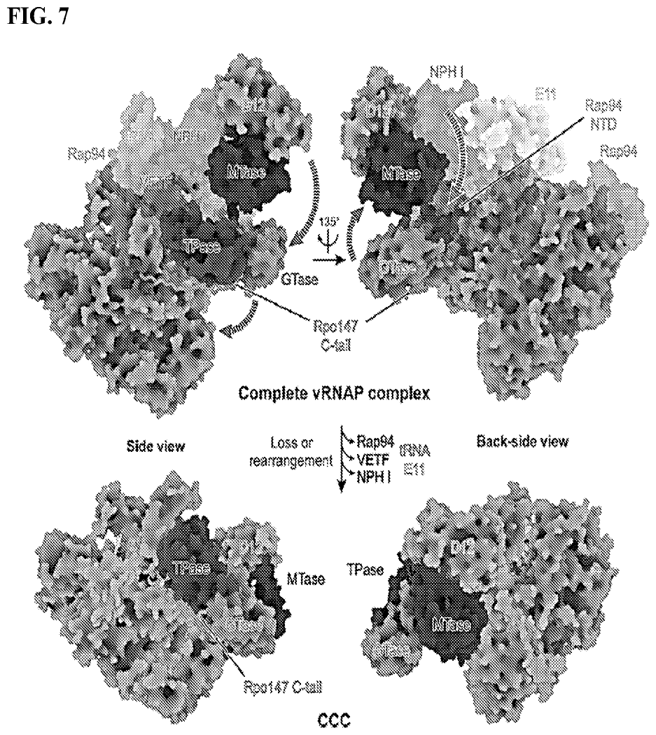

Next the CCC was compared to the complete vRNAP structure reported in the accompanying Example (Grimm et al.). The CCC lacks the viral transcription factors observed in the complete vRNAP complex. Despite extensive classification efforts, particle populations containing these transcription factors could not be detected in the dataset (FIG. 11). In the complete vRNAP, the orientation of CE relative to the vRNAP core differs substantially, as does the relative position of the two CE modules (FIG. 7). The GT/TP module is located on the same face of the polymerase near the Rpo18 stalk but is rotated by ˜90° and swung away from vRNAP. The MT/D12 module is hinged upward, rotated and placed away from the polymerase surface. The different arrangement of the two CE modules in the complete vRNAP structure is stabilized by the N-terminal domain of the transcription factor Rap94, which forms a wedge between the two modules. Thus, formation of the active CCC described here involves displacement of Rap94, which allows for a rearrangement of CE and its docking to the vRNAP surface around the exiting RNA substrate.

Repositioning of Capping Enzyme Intermodule Linker

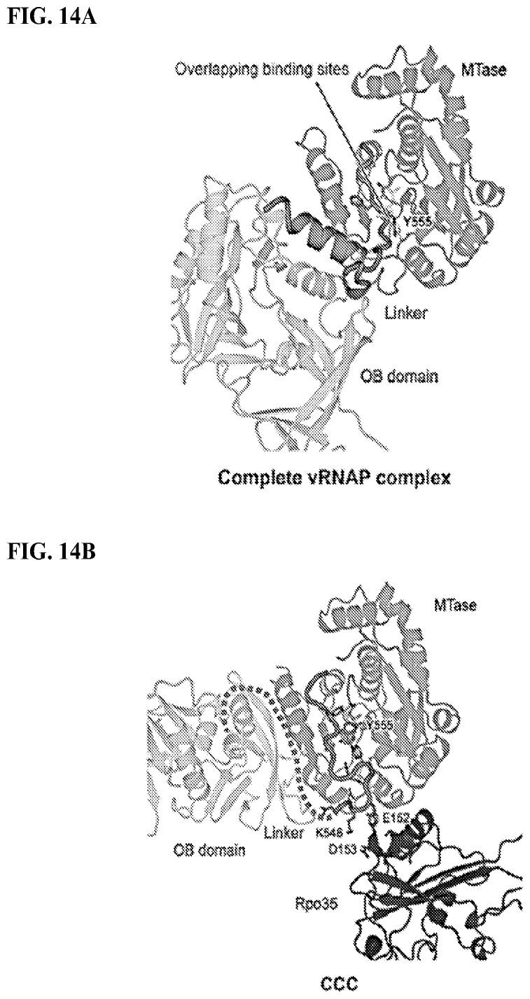

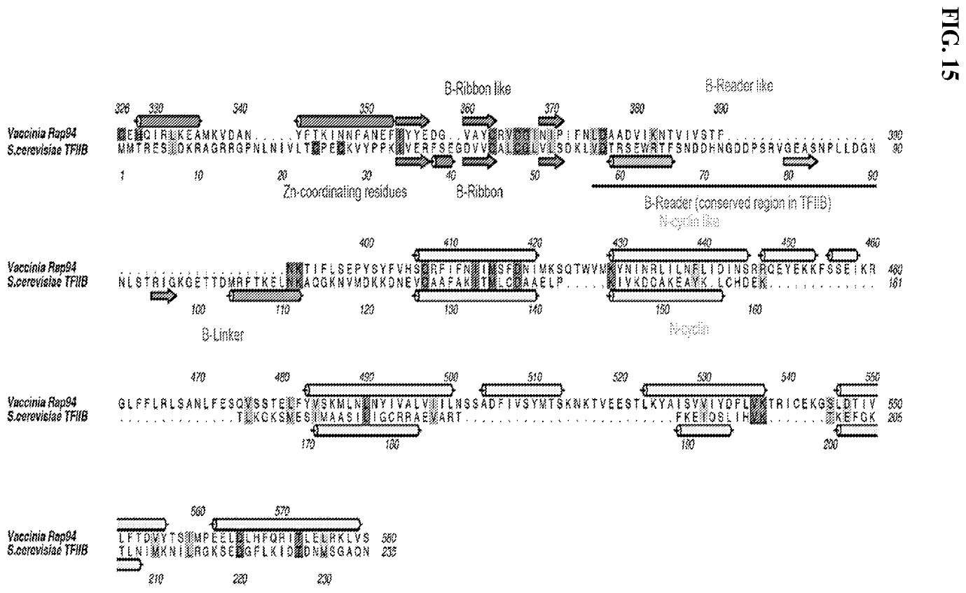

Comparison of the CCC structure to the complete vRNAP complex also reveals a repositioning of the intermodule linker that connects the two CE modules (D1 res. 530-560). The intermodule linker is ordered in the complete vRNAP complex (Example 3). Residues 550-560 are located near the MTase active site and Y555 occupies the site for the adenine base in the SAM cofactor (FIG. 14A). Thus, the intermodule linker sterically interferes with binding of the SAM cofactor to the MTase. In the CCC structure, however, the interdomain linker is partially displaced, appears to interact with a Vaccinia-specific region of Rpo35, and adopts a conformation that is now compatible with SAM binding to the MTase (FIG. 14B). This position of the intermodule linker residues 545-560 corresponds to that previously observed in crystal structures (Kyrieleis et al., 2014; la Peña et al., 2007). The linker was also previously shown to contribute to SAM binding (la Peña et al., 2007). Taken together, the interdomain linker likely contributes to an inactivation of CE in the complete vRNAP and its displacement and repositioning in the CCC is required to convert the CE to a fully active conformation.

The C-Tail of Rpo147 is a Spring-Like Tether for CE

Although the CE is present in the complete vRNAP complex, its location and orientation differ from that observed in the CCC (FIG. 7). In the complete vRNAP structure, the extensive interactions between CE and vRNAP that are observed in the CCC structure are not observed. The only CE-vRNAP contact present in the complete vRNAP complex is an interaction with the Rpo147 C-tail (res. 1259-1286). This C-tail undergoes a folding transition during the major rearrangements of CE that occur during conversion of the complete vRNAP into the CCC. In particular, the C-tail adopts an extended conformation in the complete vRNAP structure (Grimm et al., submitted in parallel), whereas it adopts an alpha helical conformation in the CCC (FIG. 7). This suggests that the C-tail of Rpo147 forms a flexible tether for CE, acting like a loaded spring that could help to pull the TP/GT module onto the polymerase surface during CCC formation.

Rap94 Displacement During the Initiation-Elongation Transition

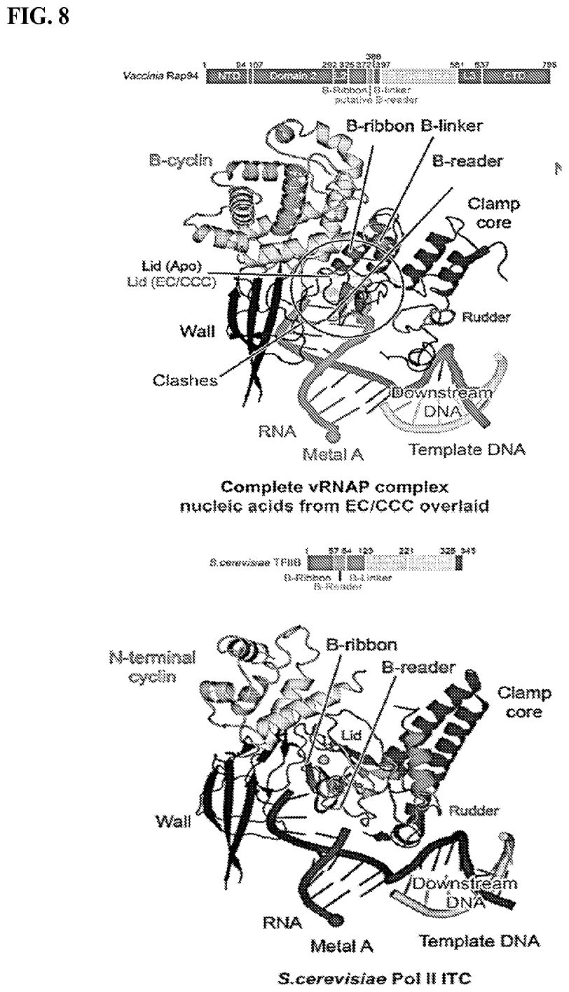

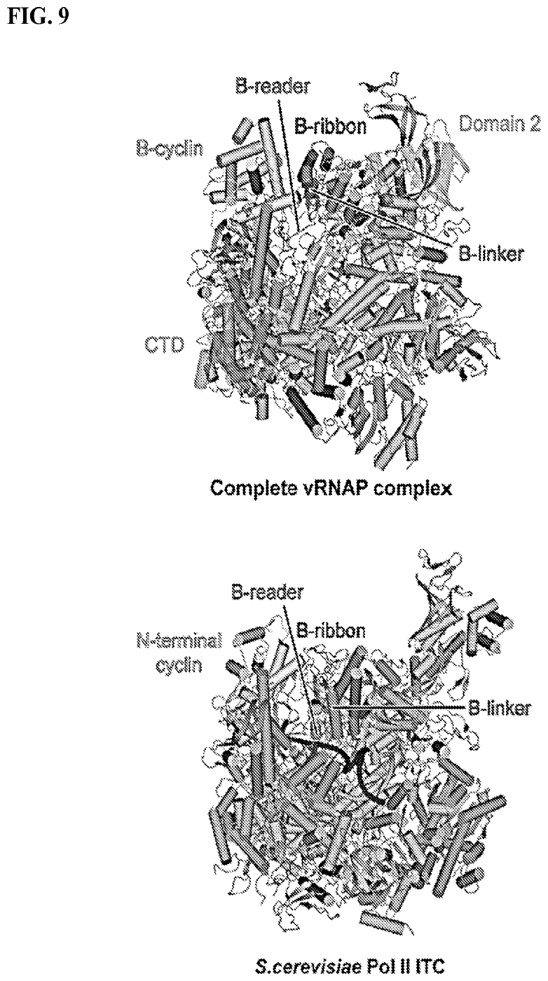

Due to steric restraints, repositioning of CE is only possible after the initiation factor Rap94 is displaced from its location in the complete vRNAP complex. This raises the question when and how Rap94 is displaced. As described in other Examples, Rap94 contains a middle domain that structurally resembles the eukaryotic general transcription initiation factor TFIIB (Grimm et al., submitted in parallel). This suggests that Rap94, like TFIIB, 15 displaced during the initiation-elongation transition. Indeed, structural comparisons between the CCC and the complete vRNAP complex indicate that the growing RNA transcript displaces Rap94 from the vRNAP surface, similar to displacement of TFIIB from Pol II upon RNA extension (Kostrewa et al., 2009; Sainsbury et al., 2013) (FIG. 8 and FIG. 9). When the RNA grows to a length of 7-8 nt, it would clash with the B-reader element of Rap94, which is reduced compared to TFIIB (FIG. 15). When the RNA grows to a length of about 12 nt, it would also clash with the B-ribbon domain of Rap94. In addition, the upstream DNA duplex in the CCC structure resides at the location occupied by the B-cyclin domain of Rap94, also requiring Rap94 displacement upon EC formation. These observations show that elongation of the RNA transcript beyond a critical length leads to clashes with the B-homology region of Rap94, which are predicted to displace Rap94 from the vRNAP surface, and to reposition the CE around the RNA exit tunnel.

Binding of Rap94 and Nucleic Acids is Mutually Exclusive

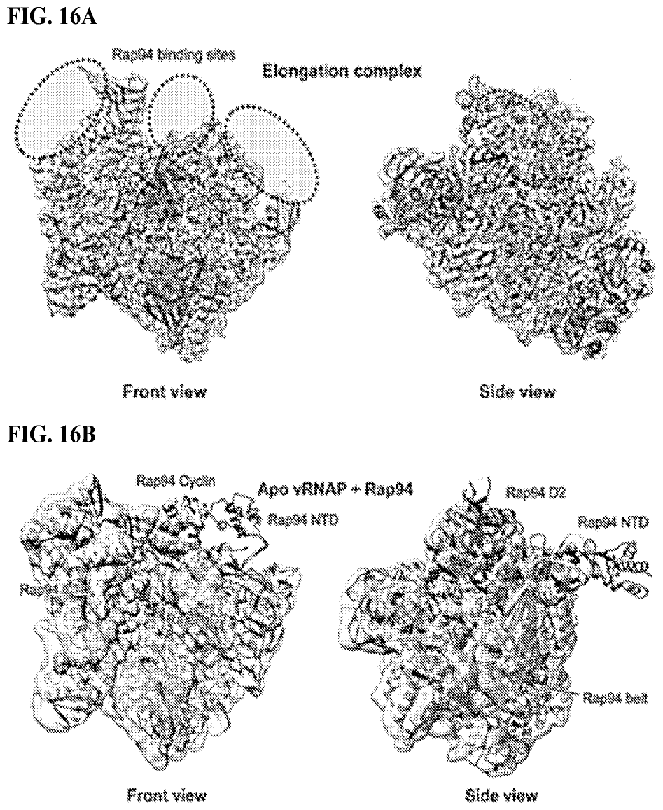

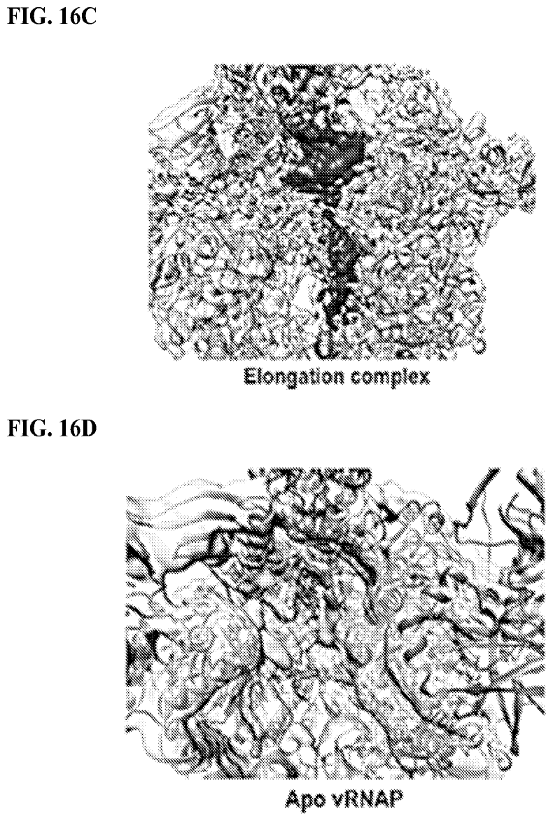

The model for the initiation-elongation transition described above predicts that the active center of vRNAP can either accommodate the B-homology region of Rap94 or the DNA-RNA hybrid, but not both. Evidence for this comes from a further classification of the cryo-EM data for the EC (FIG. 11). A fraction of particles lacking nucleic acids was sorted out, which led to a reconstruction at an overall resolution of 4.2 Å (FIGS. 11 and 12). This reconstruction showed density for Rap94, including the B-homology region, but lacked the DNA-RNA hybrid (FIG. 16). This shows that the absence of Rap94 from the EC and CCC structures cannot be attributed to a lack of the factor from the sample. Instead, Rap94 is present in the sample and must be displaced from vRNAP when nucleic acids bind to induce a functional state of the enzyme.

### DISCUSSION

Here is provided detailed structural information on Vaccinia virus transcribing complexes in two different forms. The structure of the elongation complex (EC) reveals that the nucleic acid arrangement in the active center is highly similar to that observed in cellular multisubunit RNA polymerases, indicating the same general mechanism of DNA-dependent RNA synthesis. The structure of the co-transcriptional capping complex (CCC) provides the first high-resolution snapshot of co-transcriptional capping and reveals how the RNA substrate binds the triphosphatase (TPase) active site. Together with published functional information and the structures of free vRNAP reported in the accompanying Example (Example 3), the results elucidate viral transcription mechanisms and suggest the nature of the rearrangements that occur during the transition from transcription initiation to elongation.

From the available data, the following model of Vaccinia virus transcription emerges. First, vRNAP engages with the promoter DNA duplex, and this is mediated by the initiation factors Rap94 and VETF in a way that remains to be understood structurally (Broyles and Li, 1993; Broyles and Moss, 1988; Broyles et al., 1991; Broyles, 2003; Hagler and Shuman, 1992b). The partial similarity of Rap94 with the Pol II initiation factor TFIIB indicates that aspects of promoter binding resemble this process in the Pol II system, where TFIIB positions the DNA above the active center cleft of the polymerase (Kostrewa et al., 2009; Plaschka et al., 2016; Sainsbury et al., 2013). The DNA is then opened, and the template strand inserted into the active site, where it may interact with the Rap94 B-reader and B-linker elements. During open promoter complex formation, the Rpo30 C-tail must liberate the active center, and this could lead to a repositioning of the B-reader. RNA synthesis can now commence and leads to a displacement of Rap94 when the RNA reaches a critical length and interferes with the B-homology region of Rap94 that occupies the RNA exit tunnel.

Displacement of Rap94 also frees the polymerase surface that binds the capping enzyme (CE). The CE can now dock near the RNA exit tunnel, and this involves a major rearrangement of its two mobile modules. As a result, the three active sites of CE are aligned around the tunnel exit where the nascent RNA 5′-end emerges from the polymerase surface. For cap formation, the RNA 5′-end must now engage with the three active sites of CE in a sequential manner. The observed conformation of CE bound to vRNAP suggests a path for sequential transfer of the RNA substrate, which remains closely associated with the transcription machinery and thus likely protected from degradation. The RNA 5′-end may easily swing from the first active site, the TPase, into the neighboring second active site, the GTase, which resides in the same CE module. The third active site, the MTase, faced away from the GTase active site in a previous structure of free CE (Kyrieleis et al., 2014). However, rearrangement of the CE modules in the CCC structure reorients the MTase active site towards the GTase and create a positively charged surface that may facilitate RNA transfer. How RNA transfer is triggered remains to be explored.

Despite limited homology between 5′-capping machineries of different taxa, the structure of the Vaccinia CCC may be relevant for understanding co-transcriptional capping in other systems. In S. cerevisiae, the first two steps of capping are carried out by a complex of two enzymes, Cet1 and Ceg1 (Rodriguez et al., 1999; Shibagaki et al., 1992; Tsukamoto et al., 1997), which are structurally similar to Vaccinia D1 (Gu et al., 2010; Kyrieleis et al., 2014). A cryo-EM reconstruction of the Pol II EC with bound Cet1-Ceg1 complex (Martinez-Rucobo et al., 2015) indicates that Cet1 binds to the polymerase in a similar location as the TP/GT module of D1, but did not reveal any details due to the low resolution. Furthermore, there is a similarity in how vRNAP and Pol II recruit CE to the polymerase surface. Whereas the C-tail of the largest vRNAP subunit tethers CE in the viral system (Chiu et al., 2002; Coppola et al., 1983; Moteki and Price, 2002), the phosphorylated CTD of the largest Pol II subunit is known to bind the CE in yeast (1997). The human capping enzyme differs from that of Vaccinia and yeast, but it is likely that topological similarities will be observed in the future, because capping also occurs already when the RNA emerges on the Pol II surface (Chiu et al., 2002; Coppola et al., 1983; Moteki and Price, 2002).

The viral transcription cycle requires the additional transcription factors Rap94, VETF and NPH-I (Broyles, 2003). The structures of functional vRNAP complexes do not reveal these factors, consistent with the finding that Rap94 is displaced when transcribing complexes are formed but required to retain these factors in the complete vRNAP structure (Grimm et al., submitted in parallel). Although Rap94 and other transcription factors are displaced from the vRNAP surface, it is possible that at least some of them remain loosely associated with the polymerase via short tail or linker regions. After 5′ cap synthesis, transcription elongation can proceed to the end of the gene, where termination is mediated by NPH-I and VTF/CE (Christen et al., 1999; Hindman and Gollnick, 2016). In the future, structural insights into initiation and termination should reveal how the virus-specific factors Rap94, VETF and NPH-I mediate these phases of the transcription cycle. Results reported here and in the accompanying paper (Grimm et al., submitted in parallel) will enable such studies and provide the molecular basis for a complete mechanistic dissection of viral RNA synthesis during Poxvirus gene expression in the cytosol.

Experimental Model and Subject Details

Human HeLa S3 cells were cultured in a 37° C. incubator equilibrated with 5% CO₂ and 95% humidified atmosphere. The cells were cultured in DMEM (Gibco) supplemented with 10% FCS and 1% Penicillin/Streptomycin.

Method Details

Isolation of vRNAP Complexes

For purification of vRNAP from infected cells, the recombinant virus GLV-1h439 containing a HA/FLAG-doubletag at the end of A24R gene was used, encoding vRNAP subunit Rpo132 (see also Grimm at al, submitted in parallel). Hela S3 cells were grown in 15-cm plates up to 80-90% of confluence and infected with GLV-1h439 with a MOI of 1.2. Cells were pelleted 24 h later and resuspended in lysis buffer (50 mM HEPES, pH 7.5, 150 mM NaCl, 1.5 mM MgCl₂, 0.5% [v/v] NP-40, 1 mM DTT, and complete EDTA-free protease inhibitor cocktail [Sigma-Aldrich]). For vRNAP purification, the extract was incubated for 3 h at 4° C. with 200 μl anti-FLAG Agarose beads (Sigma). Beads were washed four times with buffer containing 50 mM HEPES, pH 7.5, 150 mM NaCl, 1.5 mM MgCl₂, 0.1% [v/v] NP-40 and 1 mM DTT and equilibrated with elution buffer (50 mM HEPES, pH 7.5, 150 mM NaCl, 1.5 mM MgCl₂ and 1 mM DTT). The beads-bound proteins were eluted with 3× FLAG peptide and analysed by SDS-PAGE.

Preparation of vRNAP Elongation Complex

Synthetic DNA oligonucleotides (template strand: 5′-GACTTATGATCGGATAAGAGTCCAGCCAATGACAGATGCCTCATAGCC-3′ (SEQ ID NO:1); non-template strand: 5′-GGCTATGAGGCATCCCATGCGTTGAGGACTCTTATCCGATCATAAGTC-3′ (SEQ ID NO:2)) were purchased from Integrated DNA Technologies. The RNA containing a 5′-triphosphate (5′-GAGUUGUAAUAACAAGGGAAAUGUCAUUGGC-3′ (SEQ ID NO:3)) was in vitro transcribed from a modified pSP64 plasmid (Promega) containing a self-cleaving Hepatitis Delta Ribozyme (HDV) fused to the 3′ end of the sequence of interest (Müller et al., 2006). Following large-scale plasmid purification using a Maxi Prep kit (Qiagen), the plasmid was linearized with Hind III (New Englang Biolabs) and the product was purified by phenol chloroform extraction. In vitro transcription was carried out over night at 37° C. using T7 RNA polymerase (Thermo Fisher Scientific) in the supplied buffer in the presence of 100 μg linearized template DNA and 4 mM of each NTP. The RNA was precipitated with isopropanol and purified by gel electrophoresis on a 10% denaturing polyacrylamide gel. RNA visualization by UV shadowing revealed two closely co-migrating bands corresponding to the expected product size after HDV-cleavage, and the major product was excised from the gel. The RNA was extracted in 0.3M Sodium acetate (pH=5.2) and precipitated with isopropanol. Residual salt was removed using a PD-10 desalting column (GE Healthcare). The 3′-terminal 2′-3′ cyclic phosphate resulting from the HDV cleavage reaction was removed using T4 polynucleotide kinase at 37° C. over night and the product RNA was further purified by phenol-chloroform extraction followed by isopropanol precipitation. The purified RNA was annealed to the template strand by mixing equimolar amounts of both in water and heating to 95° C., followed by step-wise cooling to 4° C.). (90 s/°). vRNAP was purified as described above (see also Grimm et al., submitted in parallel). To form the vRNAP-nucleic acid complexes, 4 μM of template strand-RNA scaffold were added to the FLAG-eluate and the sample was incubated at room temperature for 20 min before the addition of 8.45 μM non-template strand DNA (corresponding to a scaffold:vRNAP molar ratio of approx. 60:1). The sample was then concentrated to and further purified by sucrose gradient ultracentrifugation as described in the accompanying manuscript (Grimm et al., submitted in parallel). In brief, the native transcribing vRNAP complexes, was layered on top of a 10%-30% sucrose gradient and centrifuged for 16 h and 35.000 rpm at 4° C. in a Beckman 60Ti swing-out rotor. Gradient fractions were fractionated manually, separated by SDS-PAGE and proteins and nucleic acids were visualized by silver staining and ethidium bromide staining, respectively.

Cryo-Electron Microscopy

The fractions corresponding to the larger of two molecular weight species (15+16) were pooled and dialyzed twice against 500 ml of dialysis buffer (10 mM HEPES, pH 7.5, 150 mM NaCl, 1.5 mM MgCl₂, 2 mM DTT) at 4° C. using Slide-a-lyzer mini dialysis pins (20,000 MW cut-off, Thermo Fisher). The sample was diluted in an equal volume of dialysis buffer and 4 μl were applied to glow-discharged UltrAuFoil R 2/2 grids (Quantifoil) and incubated for 10 s in a Vitrobot (FEI) at 100% humidity and 4° C. prior to plunge-freezing in liquid ethane. Cryo-EM data was acquired on a Titan Krios (FEI) operated at 300 kV and equipped with a Gatan energy filter and K2 direct electron detector using a slit width of 20 eV. Movie stacks consisting of 40 frames were collected with a total dose of 40.63 electrons per Å² at a nominal magnification of 105,000×, corresponding to a pixel size of 1.05 Å/pixel.

Structure Determination and Model Building

Micrographs were processed and CTF-corrected on-the-fly using Warp (Tegunov and Cramer, 2018) and automated unsupervised particle picking was performed using a custom-trained neural network in Warp. The resulting particles were subjected to unsupervised 2D classification in Relion (Scheres, 2012; Zivanov et al., 2018) followed by initial 3D refinement using a low-pass filtered ab initio model generated in cryoSPARC as reference (Punjani et al., 2017). All subsequent steps were performed in Relion. The aligned particles were subjected to 3D classification, which resulted in two well-defined classes which differed with regard of the presence of VTF/CE. Further 3D sub-classification of each of these classes yielded homogenous particle populations of the CC and the EC, respectively (FIG. 10). Automated 3D refinement using a soft mask around the entire complex followed by per-particle CTF estimation and repeated 3D refinement yielded EM reconstructions at 3.0 Å for the EC and 3.2 Å for the CCC after post processing, respectively (FIG. 11). Resolution estimates are according of the FSC=0.143 gold standard criterion and the sharpening B-factors were automatically determined as implemented in the Relion post processing algorithm. In addition to these two reconstructions, sub-classification of the EC particle population revealed a small subset of particles to which neither nucleic acid nor VTF/CE was bound. 3D refinement of this particle subset did not reach high resolution due of the low number of particles, but the resulting density allowed for docking of the known structures and revealed the nucleic acid-free core vRNAP with Rap94 bound (FIG. 11).

The structure of the EC was modelled by placing the previously determined core vRNAP structure (Grimm et al., submitted in parallel) in the density, followed by rigid body fitting and real space adjustment in Coot (Emsley et al., 2010). An initial model of the nucleic acid was obtained by superimposing the mammalian Pol II elongation complex structure (PDB 5FLM) (Bernecky et al., 2016) and was subsequently rigid body fitted and real space adjusted in Coot. Notably, the in vitro transcription template used encoded a 31 nt RNA with a 10 nt complementary stretch to the template strand (see above). Based on several observations, however, it was concluded that the RNA present in the elongation and capping complexes is likely only 30 nt long, lacking the 3′-most nucleotide: (1) Denaturing gel electrophoresis following in vitro transcription revealed two closely co-migrating bands (not shown), suggesting 1bp-heterogeneity. This could be a result of Hepatitis Delta Virus ribozyme mis-cleavage, which was fused of the 3′ end of the RNA in the in vitro transcription template. However, the smaller product represented the vast majority and was thus selectively excised. (2) When incubated with synthetic RNAs representing either the 30- or 31-nt RNA, vRNAP displays backtracking activity on the 31 nt template, but not the 30nt template, suggesting that the 31 nt RNA is cleaved at the 3′ end by the enzyme (data not shown). (3) Although not unambiguous at the resolution obtained, the fit of the cryo-EM density is more consistent with the proposed 30 nt RNA from which the 3′-most nucleotide is absent. The quality of the density rapidly declined after the point of strand separation of the 5′ end of the RNA from the template strand, and thus no further modelling was performed. The EC structure was real space refined using phenix.real_space_refine (Adams et al., 2010) and shows excellent stereochemistry.

The CCC structure was modelled by first fitting the EC structure into the CC cryo-EM reconstruction using UCSF Chimera (Pettersen et al., 2004). This revealed a large, unmodeled density around the back of vRNAP, which could be unambiguously fitted with the TP/GT module of the previously reported VTF/CE crystal structure (PDB ID 4CKB) (Kyrieleis et al., 2014). The MT/D12 module had to be substantially rotated and translated to accommodate the remaining density. The structure was then rebuilt manually in real space in Coot. In addition to the previously observed DNA-RNA hybrid in the active site, the cryo-EM density allowed modelling of three additional bases past the point of strand separation at the upstream edge of the transcription bubble. While the trajectory of the entire nascent transcript was clearly visible in the unsharpened cryo-EM density, the quality of the B-factor sharpenend (Relion) or denoised (Warp) map was not sufficient for atomic modeling in the region between RNA residues 5 and 18, indicating conformational flexibility. Despite extensive efforts, the density for this region could not be improved by focused classification and refinement procedures. The length of the unmodeled RNA region (14 nt) suggests that it may be scrunched, which would explain the lower quality density for this region due to mobility. RNA residues 1-4, which engage in interactions with the CE Tpase barrel, showed well-defined density and thus allowed for atomic modelling. Similar as of the RNA, density of the D12 loop 116-124 in vicinity of the nascent transcript was weak, suggesting some mobility of this loop. Based on the density and comparison of the Cet1 crystal structure (Lima et al., 1999), the 5′ end of the RNA was modelled as a diphosphate product complex, as described in the main text. The CE interdomain linker showed weak density for the region 549-560, but comparison to the previous crystal structures clearly indicated an identical location of this helical fragment (Kyrieleis et al., 2014; la Peña et al., 2007) and sidechain Y555 was thus modelled as in these structures, although it lacked clear sidechain density in the EM reconstruction. Importantly, due to the clear density of the peptide backbone in this region, it cannot occupy the SAM binding site as observed in the complete vRNAP complex (Grimm et al., submitted in parallel). The CCC structure was real space refined using phenix.real_space_refine (Adams et al., 2010) and shows excellent stereochemistry.

Figures were created with PyMol (Schrodinger, LLC, 2015) and UCSF Chimera (Pettersen et al., 2004). Angular distribution plots were created with Warp (Tegunov and Cramer, 2018).

### Example 3. Structural Basis of Poxvirus Transcription: Vaccinia RNA Polymerase Complexes

Poxviruses encode a multi-subunit DNA-dependent RNA polymerase (vRNAP) that carries out viral gene expression in the host cytoplasm. Reported here are cryo-EM structures of core and complete vRNAP enzymes from Vaccinia virus at 2.8 Å resolution. The vRNAP core enzyme resembles eukaryotic RNA polymerase II (Pol II), but also reveals many virus-specific features, including the transcription factor Rap94. The complete enzyme additionally contains the transcription factor VETF, the mRNA processing factors VTF/CE and NPH-I, the viral core protein E11, and host tRNA^{Gln}. This complex can carry out the entire early transcription cycle. The structures show that Rap94 partially resembles the Pol II initiation factor TFIIB, that the vRNAP subunit Rpo30 resembles the Pol II elongation factor TFIIS, and that NPH-I resembles chromatin remodelling enzymes. Together with the other Examples provided herein, these results provide the basis for unravelling the mechanisms of poxvirus transcription and RNA processing.

The eukaryotic nucleus contains the machineries for DNA replication and gene transcription. Many viruses rely for their replication and transcription on factors of the host cell and therefore require at least a transient nuclear phase to ensure viral propagation. A remarkable exception amongst eukaryotic DNA viruses are the members of the Poxviridae family, whose replication and transcription are confined to the cytoplasm (Moss, 2013). These processes require virus-encoded factors for the production of mature mRNAs from the viral genome. Such cytosolic gene expression events were extensively studied for Vaccinia virus, a non-pathogenic prototype of the Poxviridae family. These studies revealed a virus-encoded multi subunit RNA polymerase (vRNAP) and an array of associated factors that ensure the expression of the viral genome (Broyles, 2003; Kates and McAuslan, 1967; Munyon et al., 1967).

Upon infection Vaccinia virus enters the cell via micropinocytosis and becomes uncoated (Chi and Liu, 2012; Moss, 2012). Whereas the viral genome is silent in these initial events, all subsequent steps of the replication cycle are dependent on viral transcription and translation processes. Poxviruses coordinate the different processes of DNA replication and virion formation through timing of expression of individual genes grouped into early, intermediate and late classes (Baldick and Moss, 1993). Accordingly, early genes encode factors involved in events that shortly follow infection, such as viral DNA replication and intermediate gene expression, whereas later processes of the infection cycle, such as virion assembly, require the expression of intermediate and late class gene products.

vRNAP consists of eight subunits encoded by early viral genes and termed according to their apparent molecular masses Rpo147, Rpo132, Rpo35, Rpo30, Rpo22, Rpo19, Rpo18 and Rpo7 (Rosel et al., 1986). These subunits show varying degrees of homology to subunits of Pol II, suggesting an evolutionary relationship with the host transcription apparatus (Table 3) (Ahn and Moss, 1992; Ahn et al., 1990; Amegadzie et al., 1992; 1991; Broyles and Moss, 1986; Knutson and Broyles, 2008). At the level of amino acid residues, the two largest subunits (i.e. Rpo147 and Rpo132) are approximately 20% identical to RPB1 and RPB2 of Pol II, respectively. To date, there is no structural information on vRNAPs and their complexes.

vRNAP has the catalytic potential to synthesize RNA in a DNA-dependent manner. However, in vivo it requires additional factors in order to become specifically directed to viral early, intermediate and late class genes. Early transcription has been studied most extensively and shown to require the heterodimeric Vaccinia early transcription factor (VETF), which interacts with early promoters upstream and downstream of the initiation site (Broyles, 1991; Broyles and Li, 1993; Broyles and Moss, 1988; Hagler and Shuman, 1992). Together with Rap94, VETF mediates the recruitment of vRNAP to its promotors and its transition into active elongation (Broyles, 2003). Rap94 has also been proposed to connect vRNAP with VETF and NPH-I to facilitate termination (Christen et al., 1999; Hindman and Gollnick, 2016; Mohamed and Niles, 2001; Piacente et al., 2003). Other virus-encoded proteins are used to add a 5′-terminal m⁷G-cap and a 3′-terminal poly(A)-tail to viral RNAs. They include the heterodimeric Vaccinia termination factor/capping enzyme (VTF/CE), consisting of subunits D1 and D12, and the termination factor NPH-I, which acts together with a poly(A) polymerase to form polyadenylated 3′-ends. Whether these factors are part of defined functional vRNAP complexes is unknown.

Here is described the isolation of two distinct vRNAP complexes from human cells infected by Vaccinia virus: The ˜500 kDa vRNAP core enzyme and the ˜900 kDa complete enzyme with six additional viral proteins plus tRNA^{Glu} from the host. The structures of these two complexes were determined by cryo-electron microscopy (cryo-EM). Whereas the core complex represents the active core RNA polymerase, the complete enzyme apparently represents the packaged machinery containing the factors for early gene transcription. The structures reveal similarities and differences between the viral cytoplasmic transcription apparatus and the nuclear RNA polymerase machinery. These results form the basis for unravelling the molecular mechanisms of poxvirus gene transcription and RNA processing, and enabled structure determination of functional vRNAP complexes as shown in the other Examples.

Results

Purification of Vaccinia vRNAP Complexes

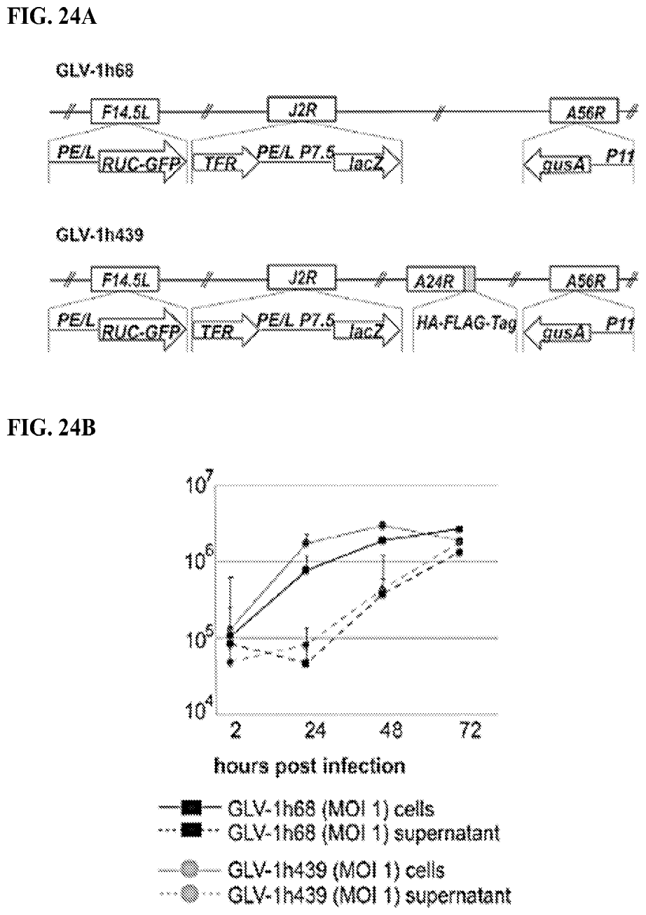

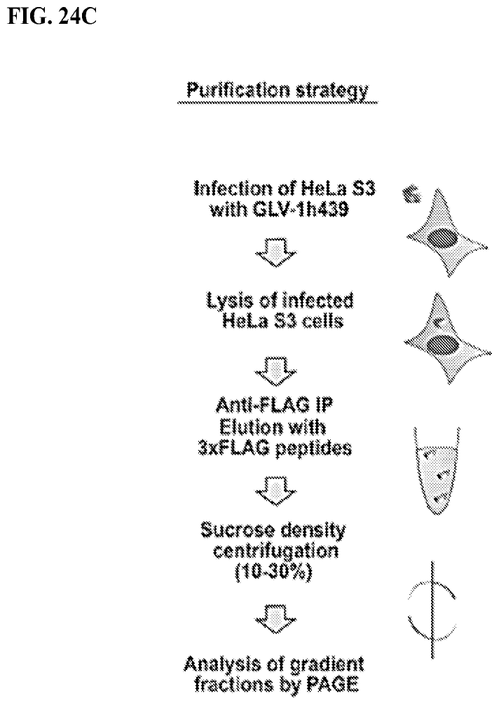

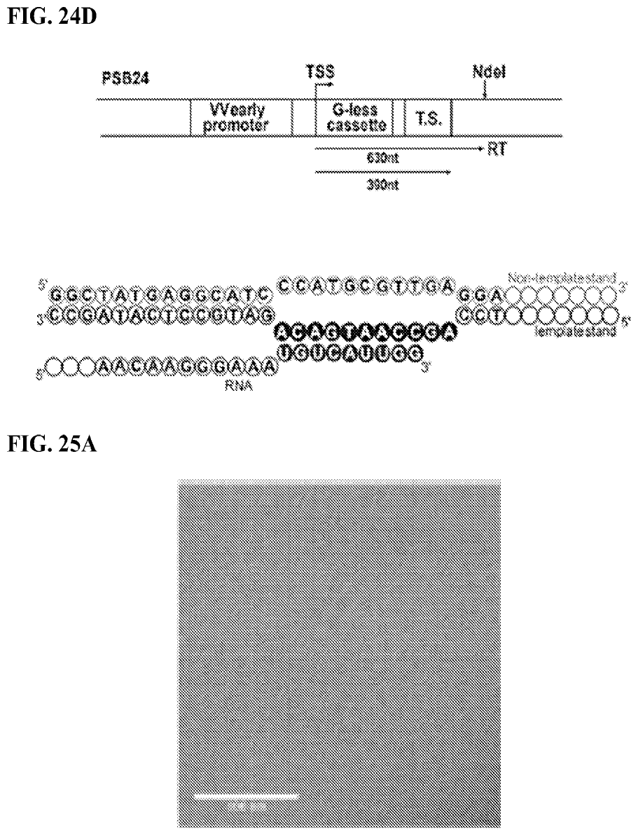

A purification strategy for the isolation of vRNAP complexes was developed based on the recombinant Vaccinia virus strain GLV-1h439. This virus is derived from the Vaccinia Lister strain GLV-1h68 and expresses a C-terminally HA/FLAG tagged vRNAP subunit Rpo132 (FIG. 24A). GLV-1h439 multiplied at similar rates as the untagged parental GLV-1h68 strain upon infection of HeLa cells, suggesting that the tag on Rpo132 does not interfere with the transcriptional activity and replication of the virus (FIG. 24B).

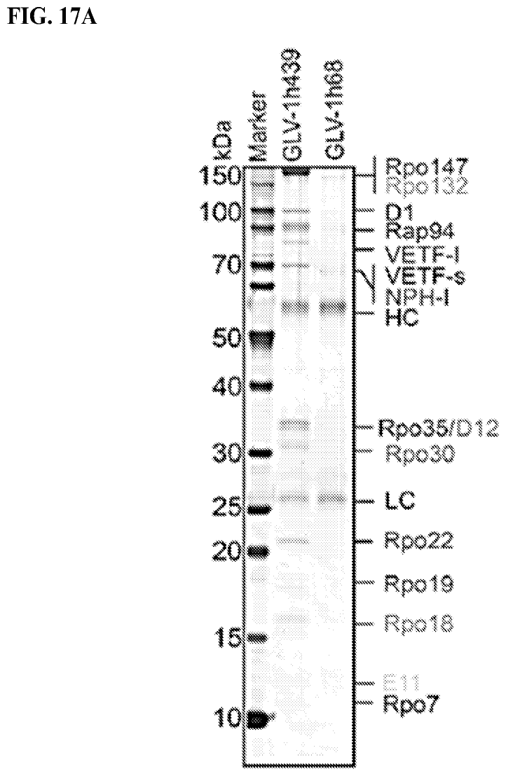

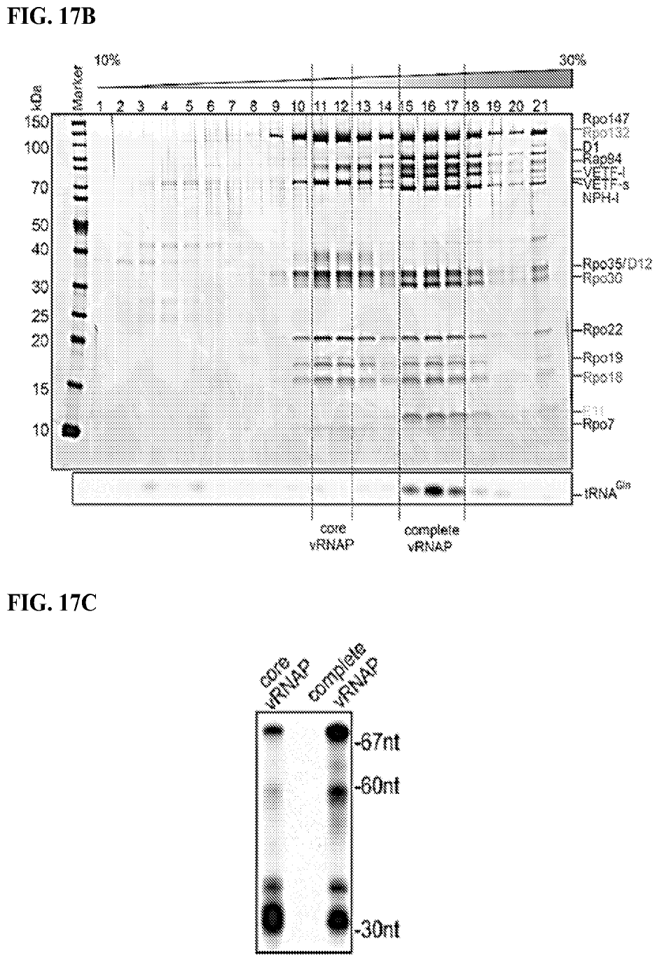

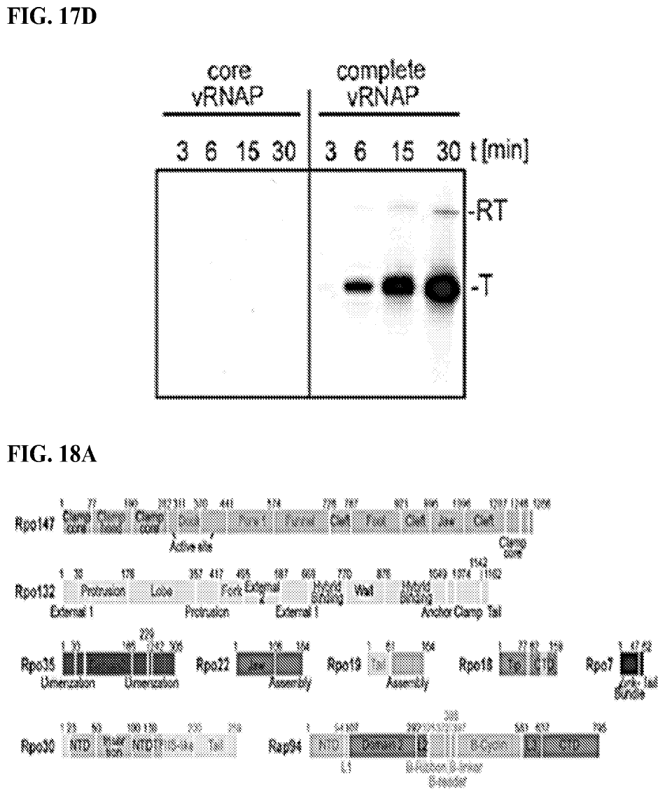

For affinity-purification of vRNAP, HeLaS3 cells were infected with GLV-1h439. Extract from infected cells was then subjected to purification on an anti-FLAG column and tagged Rpo132, along with its interacting partners, was eluted with FLAG peptide (FIG. 24C). The eluate was separated by gel electrophoresis (FIG. 17A) and analysed by mass spectrometry. All known subunits of the vRNAP core enzyme as well as the transcription factor Rap94, the capping enzyme VTF/CE (D1/D12), the termination factor NPH-I, and the early transcription factor subunits VETF-1 and VETF-s (A7/D11) were enriched in the GLV-1h439 elution. None of these factors was enriched in a control purification performed with extracts from cells infected with the untagged virus (FIG. 17A). This purification also identified the viral core protein E11L and the host tRNA^{Glu} as new factors associated with the Vaccinia virus transcription apparatus.

Vaccinia RNAP Complexes are Functional

When the eluate was analyzed by sucrose gradient centrifugation and mass spectrometry, two major complexes became apparent. The lighter complex contained all subunits of the vRNAP core enzyme including sub-stoichiometric amounts of Rap94 (FIG. 17B). Biochemical characterization revealed that this complex represents the catalytically active RNA polymerase core enzyme, as it is capable of elongating an RNA primer in vitro (FIG. 17C). However, no transcriptional activity was detected on an artificial gene under the control of a fully double-stranded viral promoter (FIG. 17D), confirming that the core enzyme requires additional factors for initiation.

The second, heavier complex contained all subunits of the core enzyme and additionally VTF/CE, NPH-I, VETF-1, VETF-s, E11L and tRNA^{Glu} (FIG. 17B). This complex was capable of early promoter-dependent transcription initiation, elongation and termination at a viral termination signal in vitro (FIGS. 17C and 17D). Taken together, the first complex represents the catalytically active core vRNAP enzyme, whereas the second complex represents a complete enzyme that comprises core vRNAP and viral transcription and RNA processing factors, and is competent of carrying out all steps of the early Vaccinia transcription cycle.

Structure of Vaccinia Core vRNAP

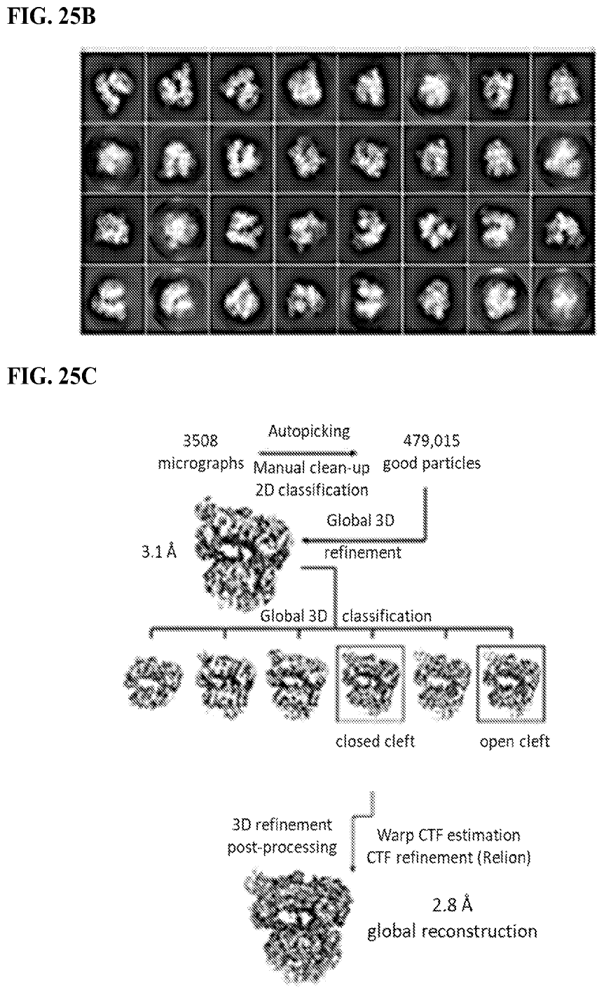

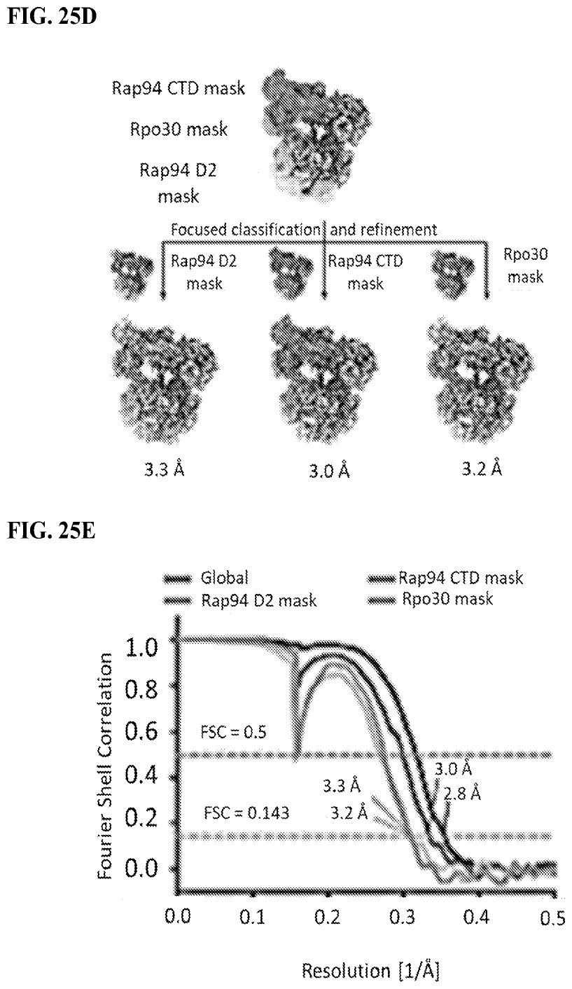

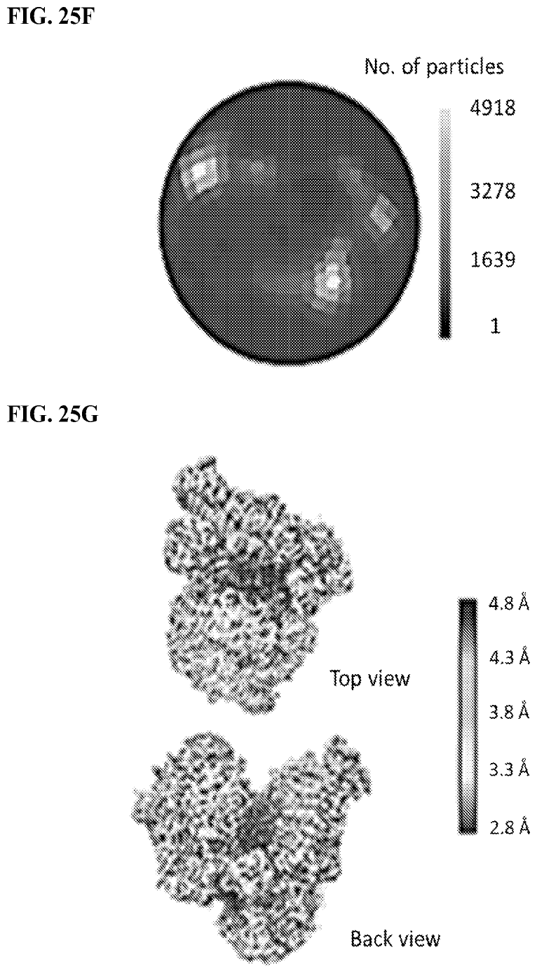

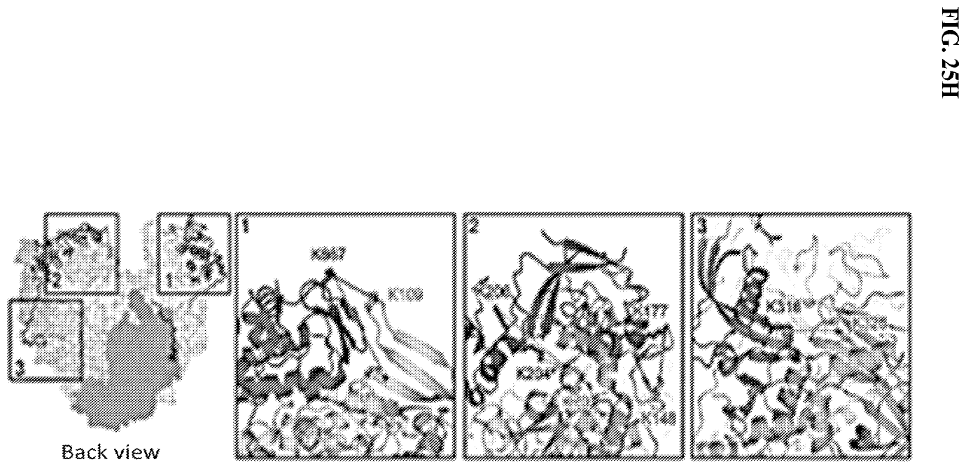

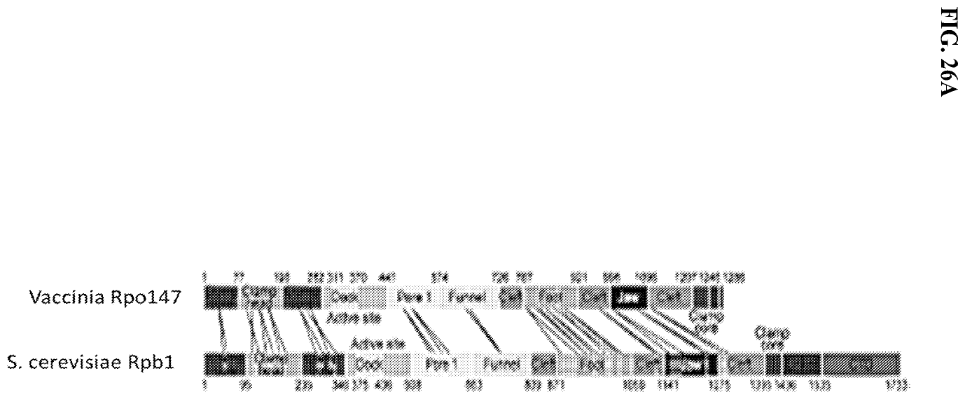

The core vRNAP was analyzed by single-particle cryo-EM and obtained a reconstruction at 2.8 Å resolution (FIGS. 25A-25G). The high resolution allowed for placement and adjustment of homology models or de novo modelling of all eight subunits. The reconstruction showed additional densities, which were found to stem from Rap94 according to chemical cross-linking (FIG. 25H). Focused classification and refinement yielded improved maps that allowed for modelling of two domains of Rap94 on opposite sides of the polymerase. The resulting structure of the vRNAP core enzyme has good stereochemical quality and contains all eight core vRNAP subunits, four structural zinc ions, the catalytic magnesium ion A, and two domains of Rap94.

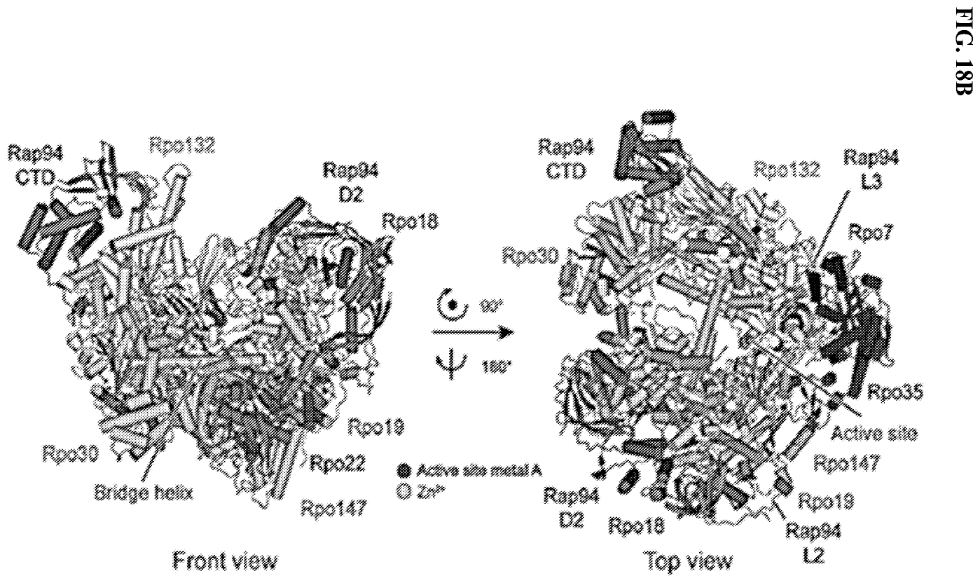

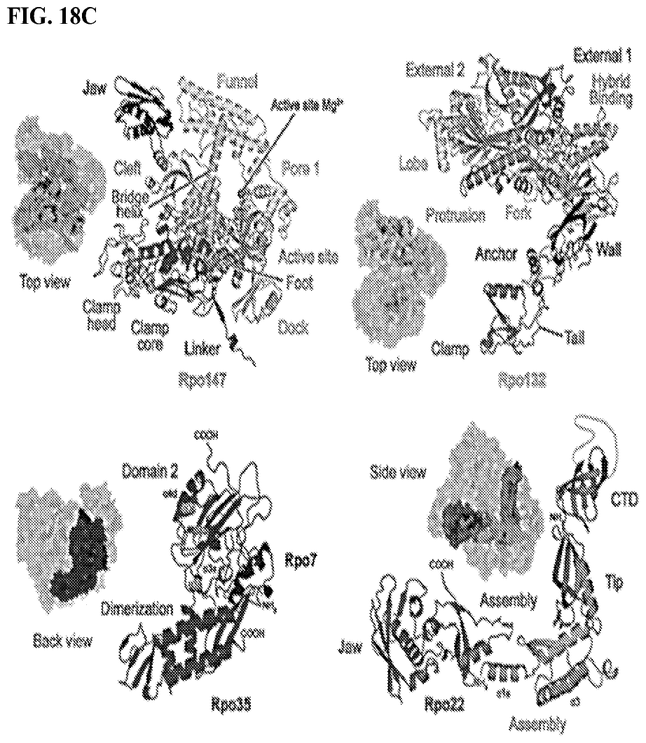

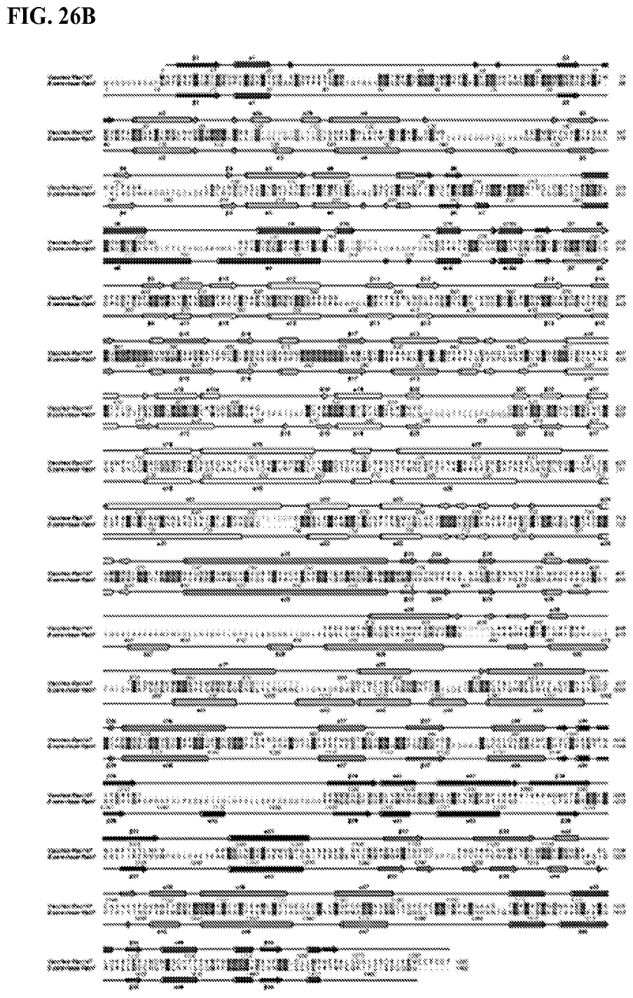

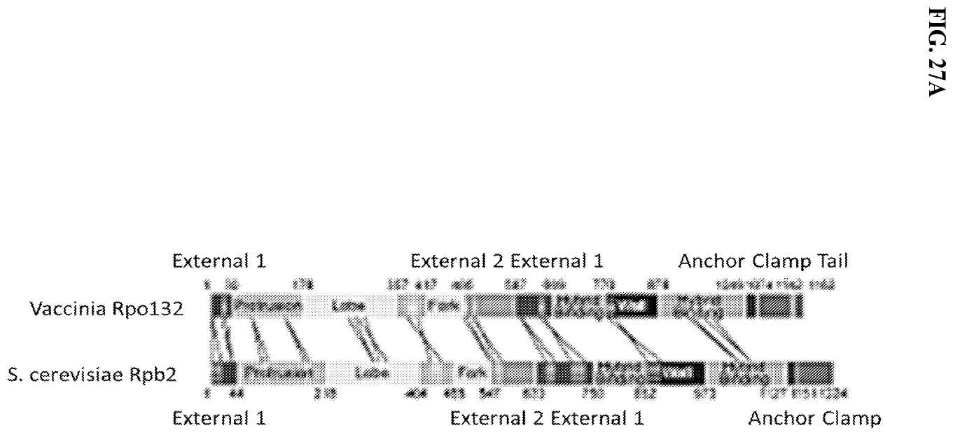

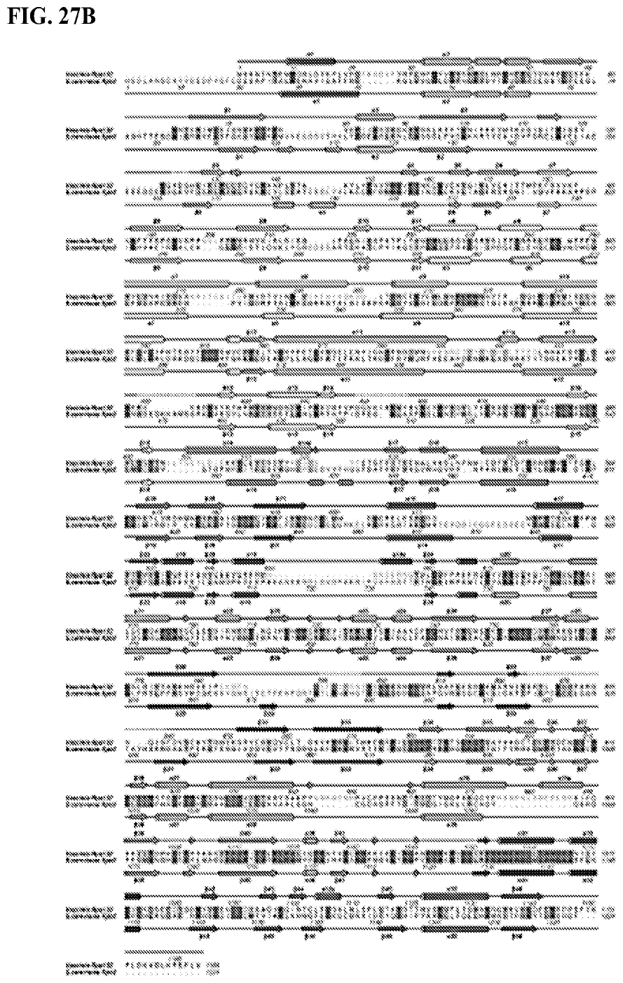

The structure shows that core vRNAP resembles multisubunit RNA polymerases in eukaryotic cells, and in particular Pol II (FIG. 18). Based on structural and sequence homology, domains in all subunits were annotated in accordance to their counterparts in S. cerevisiae Pol II, which serves as a paradigm for eukaryotic multisubunit RNA polymerases (FIG. 18, FIGS. 26-28) (Armache et al., 2005; Cramer et al., 2001; 2000). The two large subunits, Rpo147 and Rpo132, form two sides of a central cleft that holds the active center, giving vRNAP the typical bilobal appearance of multisubunit RNA polymerases found in all three domains of life (Cramer et al., 2000; Hirata et al., 2008; Zhang et al., 1999) (FIG. 18B). Subunits Rpo35 and Rpo7 form a subassembly on the back of the polymerase body that contacts both large subunits (FIG. 18C).

The entry path for the DNA duplex to the cleft is lined by two ‘jaws’ formed by Rpo147 and subunit Rpo22 (FIG. 18C). Rpo22 assembles with subunits Rpo19 and Rpo18 on the periphery of the polymerase (FIG. 18C). Rpo18 protrudes slightly from the polymerase body, forming a stalk. At its base, Rpo18 is anchored to the polymerase body and Rpo19, which in turn bridges to Rpo22. Rpo30 is only partially visible in the structure and binds with its N-terminal domain on the outside of the enzyme, near the ‘funnel’ domain of Rpo147 (FIG. 18B). The Vaccinia-specific transcription factor Rap94 is likewise only partially visible in the core vRNAP structure, with two of its domains (Domain 2 and C-terminal domain) binding to the periphery of the polymerase on opposite sides of the cleft (FIG. 18B).

vRNAP Contains a Conserved Core

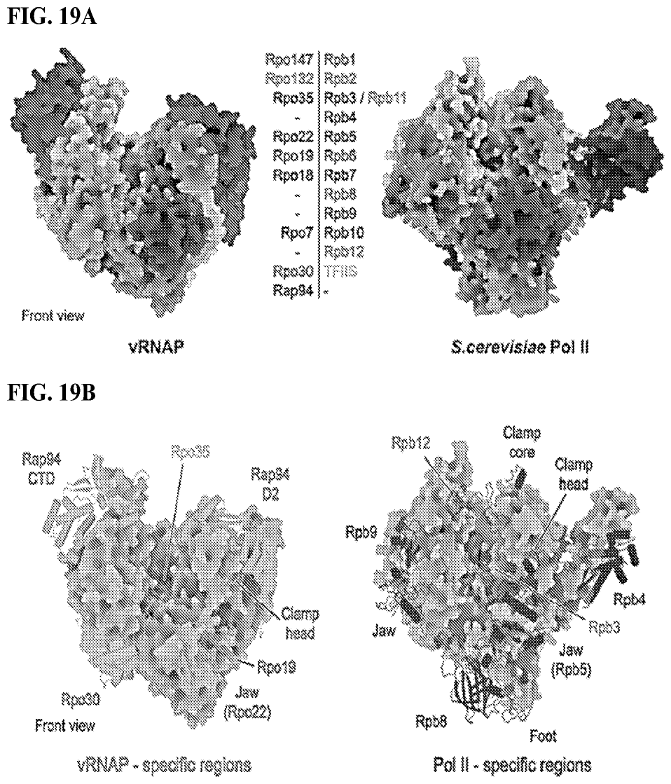

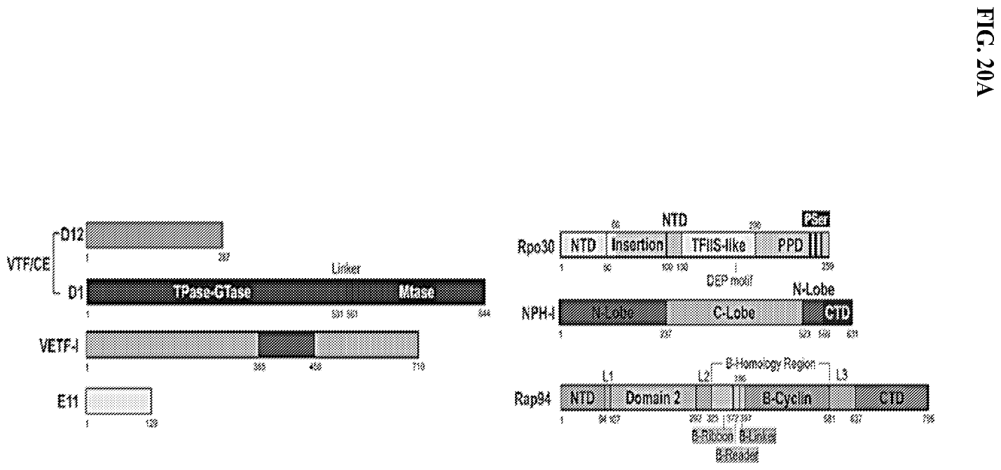

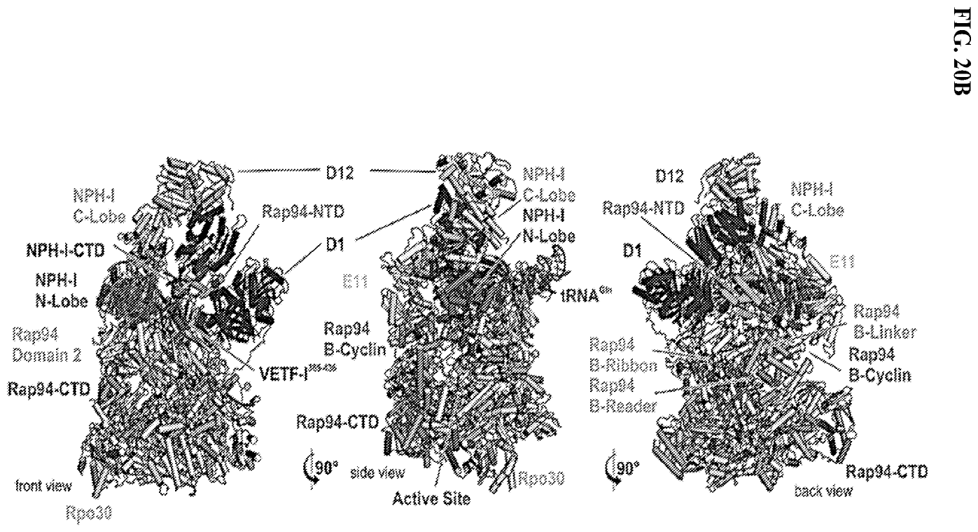

Seven of the eight core vRNAP subunits show structural homology to subunits found in Pol II, albeit their degree of similarity differs (FIG. 19A). Therefore, a structure-based comparison was carried out between vRNAP and S. cerevisiae Pol II that provides insight into the functional roles for the individual subunits of vRNAP (FIG. 19 and FIGS. 26-28) (Armache et al., 2005; Cramer et al., 2000; 2001). The two large subunits Rpo147 and Rpo132, which form the body of the polymerase, are highly similar to their Pol II counterparts Rpb1 and Rpb2, respectively (FIG. 19B and FIGS. 26 and 27). In particular, the active center and nucleic acid-binding regions are structurally conserved. The active site is formed by an invariant D×D×D motif in Rpo147 that binds the catalytic metal ion A (FIG. 18 and FIG. 26) and is flanked by the bridge helix of Rpo147 that traverses the cleft (FIG. 18B). However, both Rpo147 and Rpo132 lack several regions and are smaller compared to the yeast counterparts (FIG. 18B and FIGS. 26 and 27).

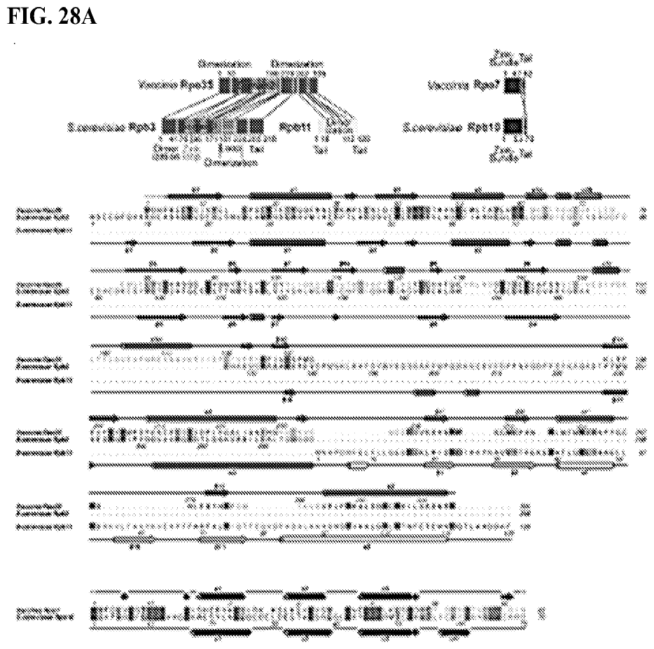

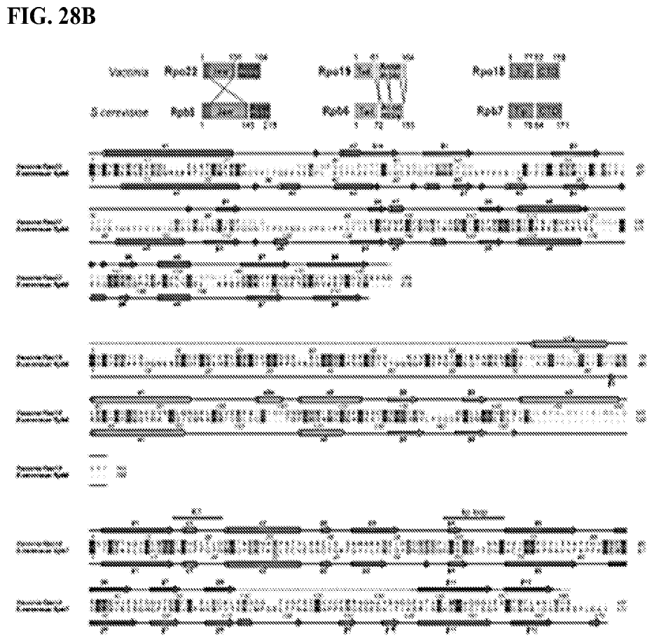

In all known multisubunit RNA polymerases, the two large subunits are anchored to a dimeric platform at the back of the enzyme, formed by Rpb3 and Rpb11 in the case of Pol II (Cramer et al., 2000; 2001; Engel et al., 2013; Fernandez-Tornero et al., 2013; Hoffmann et al., 2015). The vRNAP subunit Rpo35 combines features of both Rpb3 and Rpb11 in one polypeptide (FIG. 19A and FIG. 28). It contains a Rpb3-like N-terminal part and a C-terminal part that is similar to Rpb11. However, it lacks the zinc binding motif and the regions responsible for interactions with Rpb12 and Rpb10 in Pol II (FIG. 28A), consistent with the absence of a Rpb12-like subunit in vRNAP. The corresponding location of Rpb12 on vRNAP is instead occupied by a helical insertion in Rpo35. Rpo7 interacts with Rpo35 and closely resembles the Pol II subunit Rpb10, both in structure and location in the enzyme complex (FIGS. 18C and 28C). The C-terminal tail of Rpo7, however, extends further, forming additional interactions with Rpo35 and Rpo132. The Rpo35/Rpo7 subassembly therefore represents the viral equivalent to the Rpb3/10/11/12 subassembly in Pol II and the α₂ homodimer in bacterial RNA polymerases (Zhang et al., 1999).

Rpo22 structurally resembles Rpb5 and is located at a similar position (FIGS. 18B and 19A), as predicted previously (Knutson and Broyles, 2008). Rpo19 is a structural and functional homolog of the Pol II subunit Rpb6. As for the latter, the N-terminal tail of Rpo19 is mobile and hence invisible in the structure (FIG. 18A). The regions flanking the conserved assembly domain of Rpo19 (α1a and α3) are unique to the viral enzyme. Also, helix ala forms a contact to Rpo22 that is not observed between the corresponding Pol II subunits Rpb5 and Rpb6 (FIG. 18C and FIG. 28B). The foot domain of Rpo147 lacks some regions found in its Pol II counterpart, and this space is partially occupied by the Rpo19 ala helical insertion (FIG. 26). In summary, this detailed comparison of vRNAP to Pol II shows that the enzyme core is largely conserved between vRNAP and other multisubunit polymerases.

Vaccinia-Specific Polymerase Periphery

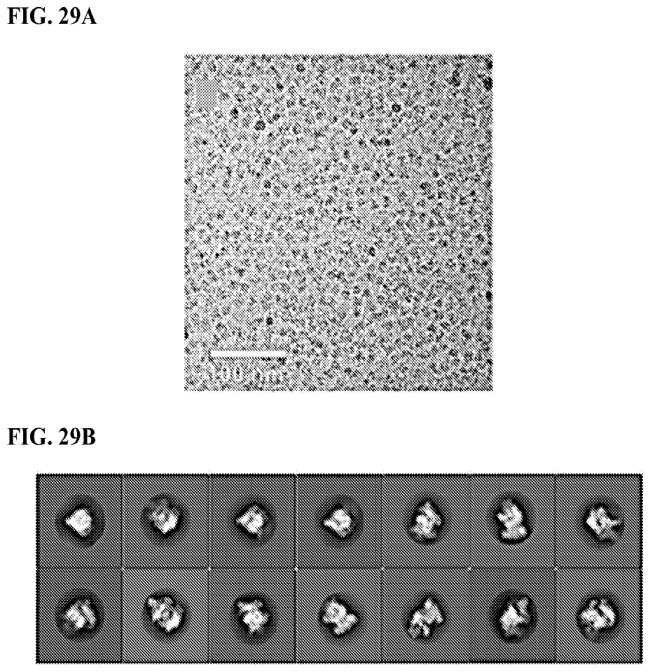

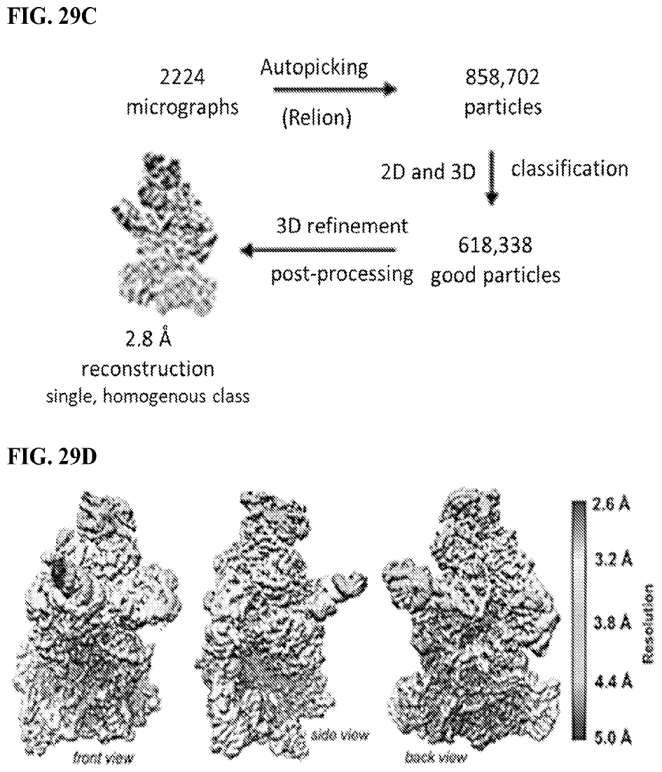

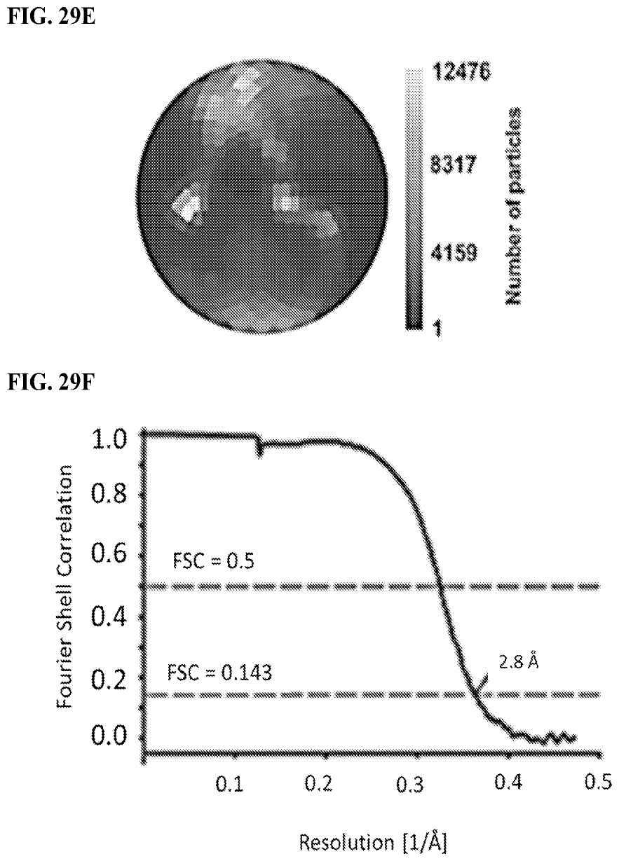

The structure-based comparison also demonstrates that the enzyme surface deviates substantially from that of other multisubunit RNA polymerases (FIG. 19B). In particular, vRNAP does not contain counterparts to the Pol II surface subunits Rpb4, Rpb8, Rpb9 and Rpb12 (FIG. 19A). Moreover, differences in related subunits of vRNAP and Pol II also map to the surface of the enzymes (FIG. 19B). For example, the clamp core domain in the largest subunit is smaller in vRNAP, but larger and involved in transcription factor interactions in Pol II (Bernecky et al., 2017; Martinez-Rucobo et al., 2011; Plaschka et al., 2016). Likewise, the jaw and foot domains in the largest subunit Rpo147 are also smaller. Rpo147 also does not possess the long and repetitive C-terminal domain (CTD) found in its Pol II counterpart Rpb1. Instead, it contains a short C-terminal tail (‘C-tail’) (res. 1259-1286) (FIG. 29B and FIG. 26), which is mobile in the vRNAP structure and hence not visible. The second largest subunit Rpo132 lacks several small regions and contains a few insertions compared to its Pol II counterpart Rpb2. It has an extended carboxy-terminal tail (‘C-tail’) that emerges from the clamp and wraps around the polymerase, traversing across subunit Rpo19 and towards the foot domain of Rpo147 (FIGS. 18C and 19 and FIG. 27).

The jaws of vRNAP, formed by Rpo147 and Rpo22, also show unique features. Whereas the C-terminal assembly domain of Rpo22 is highly conserved, its jaw domain adopts a unique fold (FIG. 18C) and lacks the ‘TPSA’-motif found in its Pol II counterpart Rpb5 that interacts with downstream DNA (FIG. 28B) (Bernecky et al., 2016). The opposite side of the jaw, formed by Rpo147, is smaller and adopts a different orientation than in Pol II. Near this domain, the unique viral subunit Rpo30 binds at the rim of the cleft (FIG. 18B and FIG. 19B). Rpo30 does not have a counterpart in Pol II, but its N-terminal domain (NTD) is located in a similar position on the polymerase as the dissociable Pol II elongation factor TFIIS (FIG. 19A), which Rpo30 has been suggested to functionally resemble based on sequence analysis (Ahn et al., 1990; Hagler and Shuman, 1993).

A prominent unique feature of vRNAP is its one-subunit stalk, formed by Rpo18, which is homologous to the Pol II subunit Rpb7. Eukaryotic nuclear RNA polymerases I, II and III and archeal RNA polymerase all contain a heterodimeric stalk (Armache et al., 2005; Engel et al., 2013; Fernandez-Tornero et al., 2013; Hirata et al., 2008; Hoffmann et al., 2015). In Pol II, the stalk is comprised of subunits Rpb4 and Rpb7 (Armache et al., 2003) and is involved in multiple protein interactions with transcription factors during different stages of the transcription cycle (Bernecky et al., 2017; Plaschka et al., 2016; Vos et al., 2018). The overall fold of Rpo18 is virtually identical to Rpb7, except for a smaller C-terminal region (FIGS. 18C and 28B). Rpo18 uses its tip domain to bind the polymerase core with conserved structural elements (FIG. 28B). The Rpo18 tip domain may restrict movement of the clamp, as proposed for Rpb7 (Armache et al., 2003). In comparison to the Rpb4-Rpb7 stalk, the C-terminal domain of Rpo18 appears tilted towards the polymerase as it protrudes from the enzyme surface (FIG. 19A). In summary, these comparisons suggest that the surface of vRNAP has evolved specialized features, likely to facilitate interactions with virus-specific transcription factors.

Transcription Factor Rap94 Spans the vRNAP Cleft

The core vRNAP structure contains the poxvirus-specific transcription factor Rap94 bound to the enzyme periphery. Rap94 may be involved in the recognition of early viral promoters (Ahn et al., 1994) and transcription termination (Christen et al., 2008). However, no structural information is available for Rap94 and sequence-based homology searches do not detect substantial homology to any known proteins. The two Rap94 domains resolved in the core vRNAP structure occupy distant locations on the polymerase surface on opposite sides of the cleft. One of these Rap94 domains, which is referred to as Domain 2 (D2), comprises residues 107-292 and binds to the top of the vRNAP clamp, interacting with both Rpo147 and Rpo132 (FIG. 18B). It is located close to Rpo18 and may stabilize the stalk in the observed orientation. It consists of a β sheet flanked by helical regions on either side and shows no structural similarity to factors known to interact with the clamp of Pol II. The carboxy-terminal domain (CTD) of Rap94 comprises residues 637-795 and is located at the lobe of Rpo132 (FIG. 18B). The CTD contacts the protrusion domain with a β sheet (res. 661-686). The fold of the Rap94 CTD does not resemble known Pol II transcription factors. The two Rap94 domains are connected via extended linkers that wrap around the polymerase like a belt (FIG. 18B). These linkers traverse the binding sites of Pol II subunits that are absent in vRNAP, including the C-ribbon domain of Rpb9 and the Zn-binding motif of Rpb12. The central region of Rap94 (res. 317-587) is not visible in the core vRNAP structure.

Structure of Complete Vaccinia vRNAP

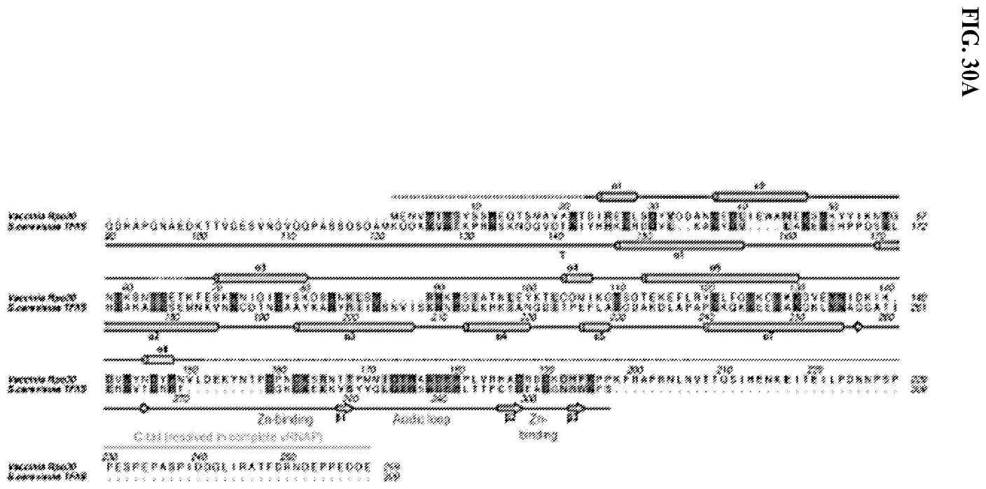

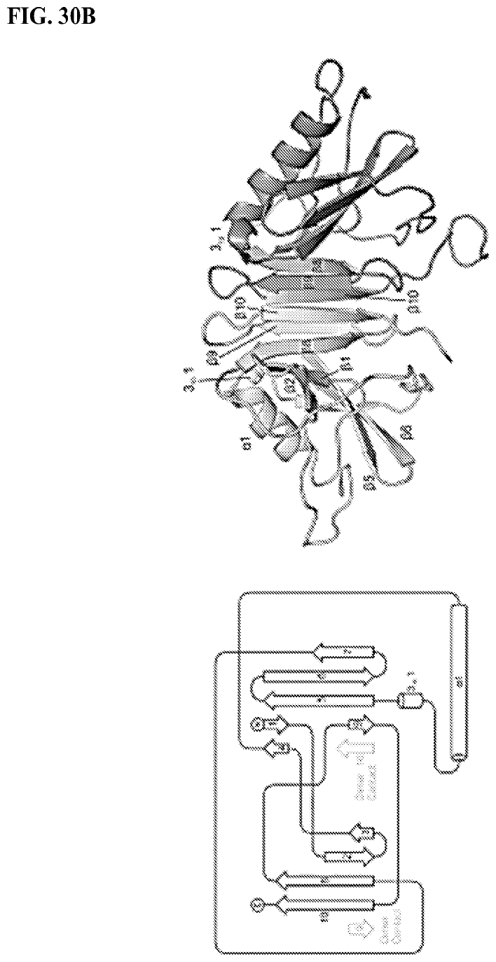

Next the structure of the complete vRNAP that contains additional transcription and RNA processing factors was determined. Acryo-EM dataset was collected from the pooled fractions 15-17 of the gradient shown in FIG. 17B, which yielded a reconstruction at 2.8 Å resolution (FIG. 29). The core vRNAP model could be unambiguously docked into the reconstruction with minor adjustments. Also, a newly determined crystal structure of the E11 core protein (FIG. 30C) was placed into the density. Then the crystal structure of VTF/CE was docketed (Kyrieleis et al., 2014). The location of the bound tRNA^{Glu} could also be identified. The remaining density regions were traced de novo and included NPH-I, the Rap94 N-terminal domain (NTD) and central region, the Rpo30 C-terminal region, a compact domain of VETF-1 comprising residues 365-436 (VETF-1³⁶⁵⁻⁴³⁶, FIG. 20), and several linker regions. The refined atomic model displays excellent stereochemistry. The complete vRNAP structure comprises 15 polypeptides and tRNA^{Gln}. It adopts an oval-shaped, bilobal structure with overall dimensions of 220 Å×150 Å×130 Å (FIG. 20B). Whereas one lobe is formed by the core vRNAP enzyme, the other lobe contains the additional factors E11, VTF/CE, NPH-I, VETF and Rap94 regions that are not resolved in the core vRNAP structure.

Rap94 Forms a Bridge Between vRNAP Core and Additional Factors

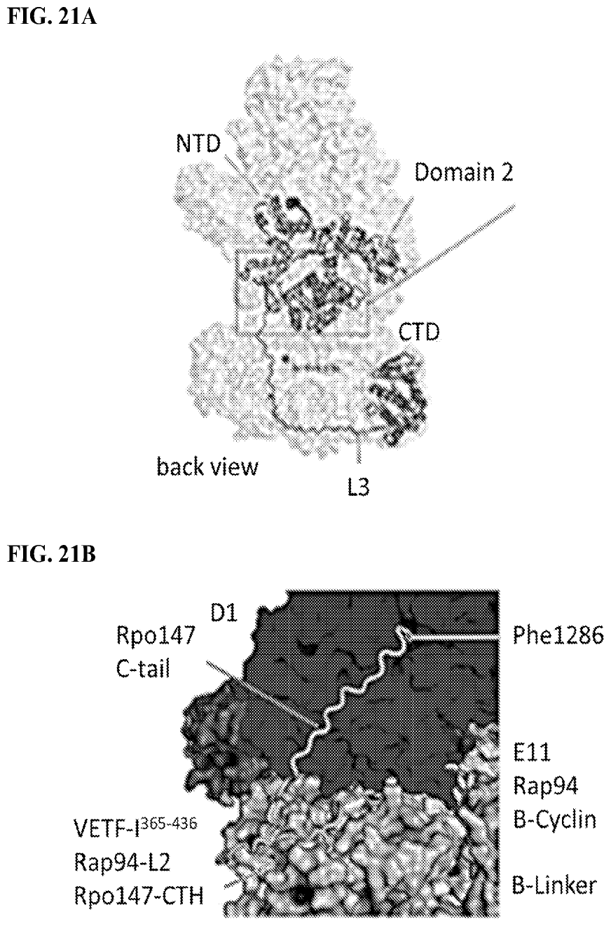

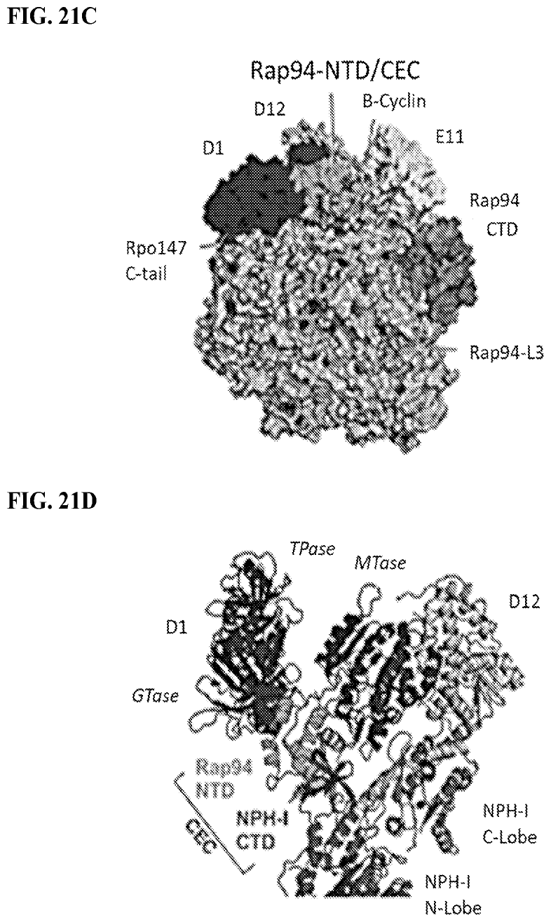

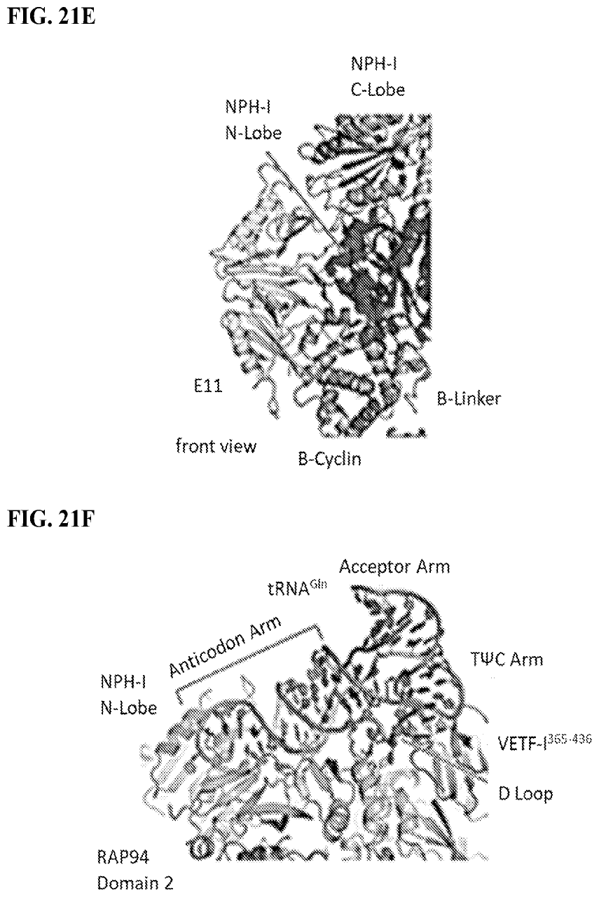

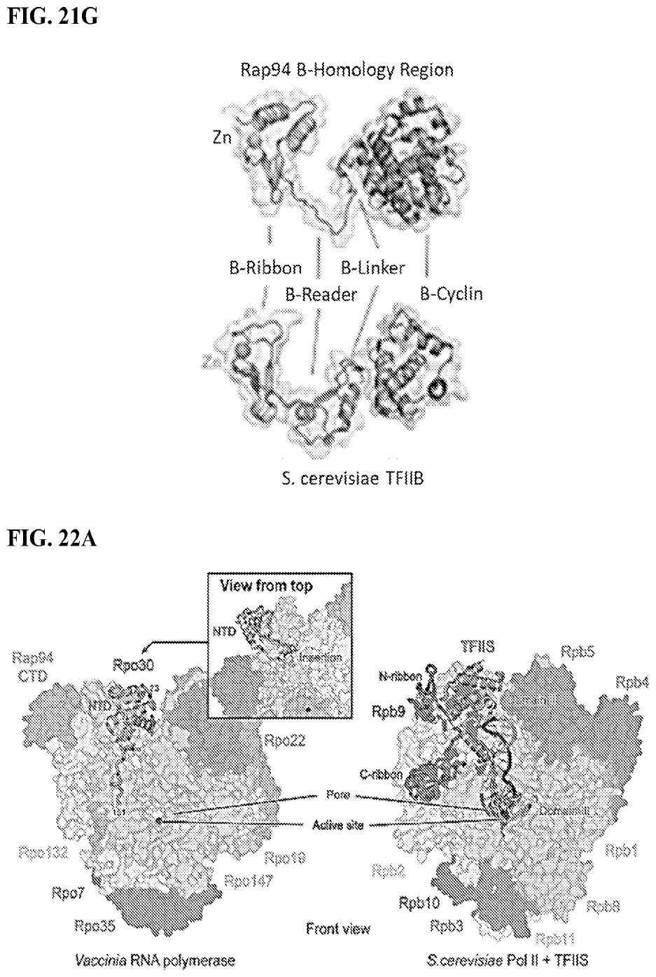

The complete vRNAP structure shows well-defined density for all parts of Rap94, which interacts with bound factors. In addition to the two domains observed in the core vRNAP structure, the NTD (res. 1-94) and the central region (res. 325-580) of Rap94 are well defined. The Rap94 domains are distributed over the entire complex and are connected by extended linker regions (FIGS. 20A and 21A). Linker 1 (L1; res. 94-107) connects the NTD to Domain 2. Linker 2 (L2; res. 292-325) emerges from Domain 2 next to the Rpo18 stalk and extends towards Rpo19, passing the C-terminal tail of Rpo147 (FIG. 21B). It then continues along the polymerase dock domain to the back of vRNAP. On the other side of the cleft, Linker 3 (L3; res. 581-637) extends near the wall and protrusion domains of Rpo132, where it traverses the binding site of Rpb12 in Pol II. L3 then extends through a groove formed by the wall and external domains of Rpo132, and to the funnel helices of Rpo147 and the Rap94 CTD (FIG. 21C).

The N-terminal region of Rap94 interacts with the C-terminal region of NPH-I. Together they fold into a domain-like module that contacts VTF/CE and that was termed the ‘CE connector’ (CEC). The CEC forms a wedge between the TPase/GTase and the MTase domains of VTF/CE, keeping the two domains apart by 10 Å compared to the VTF/CE crystal structure (FIG. 21D). A further contact between Rap94 and NPH-I is buttressed by the dimeric E11 core protein (FIG. 21E). Domain 2 of Rap94 adapts tRNA^{Glu} to the core vRNAP (FIG. 21F). In contrast to the situation in the core vRNAP structure, the C-tail of Rpo147 is ordered in the complete vRNAP and adopts an extended structure that tethers VTF/CE (FIG. 21B). Thus, Rap94 is highly modular and serves as a scaffold to assemble the complete vRNAP complex.

The Rap94 Central Region Resembles Pol II Initiation Factor TFIIB

The central region of Rap94 in the complete vRNAP (res. 325-580) is reminiscent of a large portion of the Pol II initiation factor TFIIB (FIG. 20G) and therefore it was termed ‘B-homology region’. It comprises a B-ribbon element (res. 325-371), a B-reader hairpin (res. 372-385), a B-linker (res. 386-396), and a B-cyclin domain (res. 397-580). In particular, the zinc ribbon fold and the zinc binding site in the B-ribbon are well conserved between Rap94 and TFIIB. However, the N-terminal part of the B-ribbon is formed by two unique helices in Rap94 that participate in Zn coordination via H328 instead of a cysteine. The B-linker and B-reader appear reduced compared to their TFIIB counterparts, but occupy comparable locations between the dock and clamp domains of the polymerase (Sainsbury et al., 2013). The B-cyclin domain of Rap94 corresponds to the N-terminal cyclin domain of TFIIB with respect to its fold and location. Thus the B-homology region in Rap94 occupies a similar location as TFIIB in Pol II transcription initiation complexes (Plaschka et al., 2016; Sainsbury et al., 2013), suggesting that Rap94 may function like TFIIB during transcription initiation.

Subunit Rpo30 Distantly Resembles the Pol II Elongation Factor TFIIS

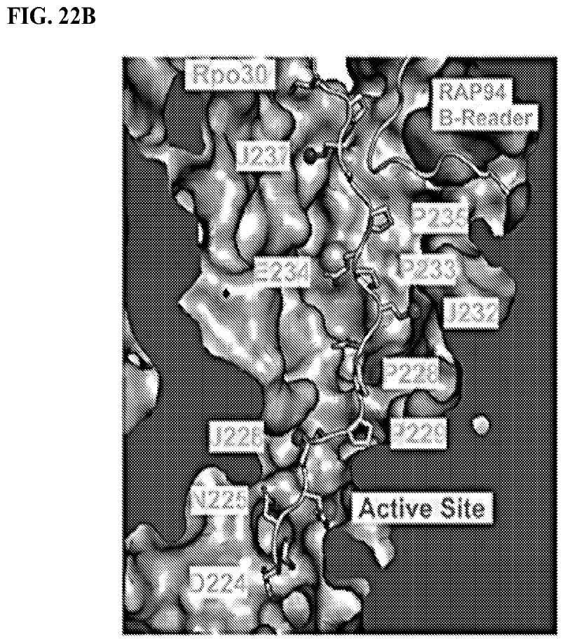

The structures show that the core vRNAP subunit Rpo30 shares similarities with eukaryotic TFIIS, as suggested based on sequence analysis (Ahn et al., 1990; Hagler and Shuman, 1993). The Rpo30 N-terminal domain (res. 23-139) binds to the rim of the polymerase funnel (FIG. 22A), at the location occupied by TFIIS domain II on Pol II (Kettenberger et al., 2003; 2004). Despite their similar location, these domains differ in sequence and structure. In particular, the Rpo30 N-terminal domain contains an insertion (res. 52-100) that wraps around the base of the jaw domain and meanders into the cleft towards the trigger loop, a mobile element of the active center (FIG. 22A, inset). The N-terminal domain of Rpo30 is connected to a linker region that extends to the Rpo147 funnel helices, forming a short single-turn helical segment (FIG. 22A).

The C-terminal domain of Rpo30 (res. 152-259) shows sequence similarity to domain III of TFIIS, a zinc ribbon that inserts into the polymerase pore to reach the active site of the enzyme (FIG. 30A) (Kettenberger et al., 2003). This domain is mobile in both of the structures, but it can likely insert into the polymerase pore and reach the vRNAP active site, as observed for domain III of TFIIS (FIG. 22A) (Kettenberger et al., 2003; 2004). This domain can trigger nucleolytic RNA cleavage at the Pol II active site, and Vaccinia vRNAP has been shown to harbour nucleolytic activity and this has been suggested to be conferred by Rpo30 (Hagler and Shuman, 1993). Thus, Rpo30 contains an N-terminal domain that binds to the polymerase in a manner reminiscent of domain II of TFIIS, and a mobile C-terminal domain that likely uses a TFIIS-like mechanism to trigger RNA cleavage at the vRNAP active site.

Rpo30 Places its Phosphorylated C-Tail in the Active Center

Rpo30 additionally contains a C-terminal tail (C-tail; res. 207-259) that is not resolved in the core vRNAP structure but is clearly visible in the complete vRNAP structure (FIG. 30A). This tail inserts into the pore of the polymerase, running past the active site and into the region that is predicted to interact with the DNA-RNA hybrid at the floor of the active center cleft (FIG. 22B). The interactions that hold the C-tail in place are centered around three phosphorylated SP sequence motifs for which clear density peaks were found that allowed for obtention of an atomic model for this Rpo30 region. Although the function of the Rpo30 C-tail remains unknown, structural superposition with a Pol II elongation complex (Gnatt et al., 2001) show that it may interfere with binding of the DNA-RNA hybrid and thus impair formation of a transcribing complex. In the accompanying paper (Hillen et al., submitted in parallel) it was shown that the DNA-RNA hybrid indeed binds at the expected position and may clash with the Rpo30 C-tail. This suggests that the Rpo30 C-tail must be displaced for transcription.

Termination Factor NPH-I Resembles Chromatin Remodelers

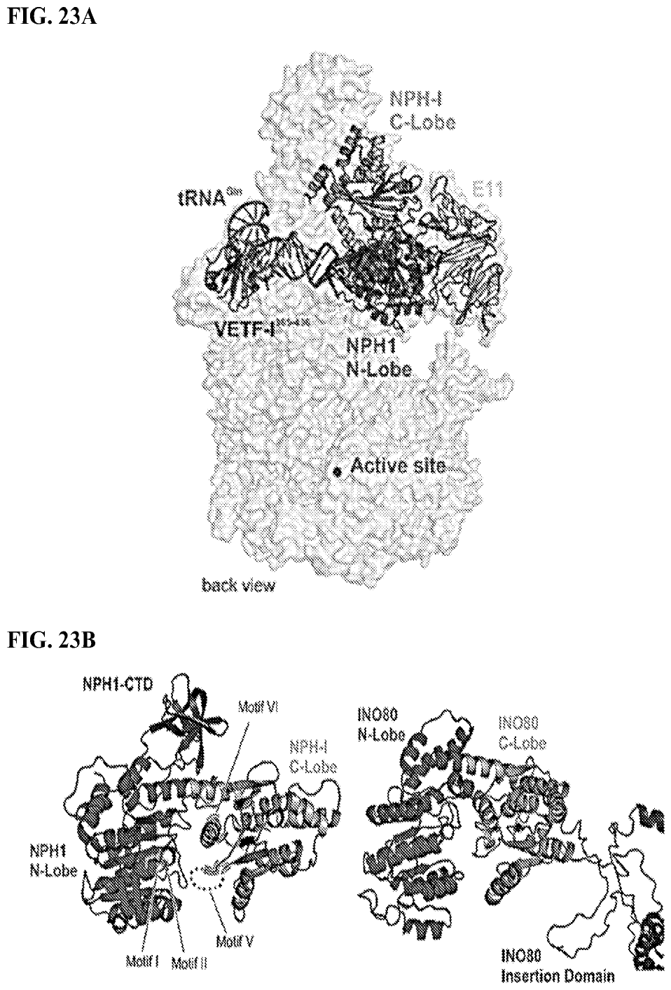

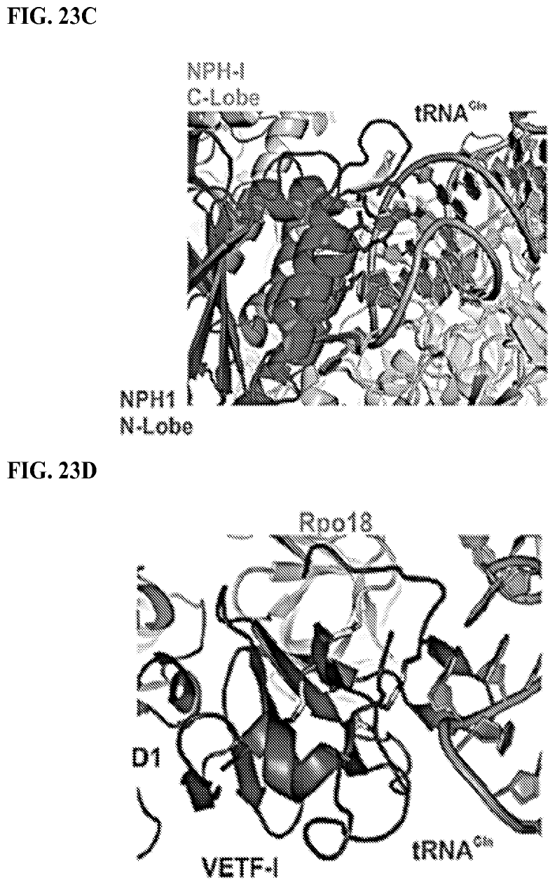

The complete vRNAP structure also contains the Vaccinia termination factor NPH-I, consisting of N- and C-terminal domains (N-lobe and C-lobe, respectively). NPH-I is located with its N-lobe near the RNA exit pore of vRNAP (FIGS. 20B and 23A). A structural homology search shows a striking similarity to the chromatin remodeler INO80 of the SNF2 family (Eustermann et al., 2018) (FIG. 23B), confirming previous predictions (Henikoff, 1993). SNF2 family proteins are ATP-driven motors with two lobes that are connected by one (INO80, FIG. S7B, middle panel) or two (SNF2, FIG. S7B, right panel) extended ‘brace’ helices, and two protrusions that facilitate DNA interactions. The lobes of NPH-I are connected by a single brace helix, and the C-lobe contains the ‘protrusion II’ found in members of the SNF2 family (FIG. 30B, left panel). An additional common feature is the surface at the inside of the ‘brace’ formed by the two helicase domains, which is lined by stretches of conserved amino acid motifs denoted as motif I-VI (FIG. 30B, left panel). The motif II (Walker B) sequence qualifies NPH-I as a DExH helicase and is strictly conserved over all members of the poxviridae family (Deng and Shuman, 1998). NPH-I additionally contains a unique C-terminal region (res. 561-639) that contacts the NTD of Rap94 as part of the CEC through multiple interactions, including an inter-protein β-sheet. NPH-I may therefore have evolved from a common ancestor of the SNF2 family and has adapted to its virus-specific function by the acquisition of its C-terminal domain.

Host tRNA^{Glu} is an Integral Component of the Complete vRNAP

A peculiar feature of the complete vRNAP complex is the presence of the host tRNA^{Gln}. RNA sequencing identified the isoacceptor tRNAs GlnTTG and GlnCTG as the predominant species. Therefore, the tRNA was modelled as tRNA-GlnTTG (chr17.trna16-GlnTTG, termed tRNA^{Glu}). The binding site of this tRNA molecule is located on the periphery and the acceptor arm points away from the center of the complex (FIG. 20B). Only weak density could be detected for the acceptor arm of the tRNA, as it is not supported by any protein contacts and hence partially mobile. tRNA^{Glu} contacts Domain 2 of Rap94, which forms a broad interface with the anticodon- and D-arm (FIG. 21F). This interaction displays no prominent contacts to particular bases in this area and hence does not confer binding specificity. However, the anticodon loop of tRNA^{Gln} is oriented in a manner that it can be specifically read out by the NPH-I N-lobe (FIG. 23C) and VETF-1³⁶⁵⁻⁴³⁶ (FIG. 23D), which may confer specificity for tRNA^{Gln}. Due to the many observed interactions of tRNA^{Glu}, it is likely important for the stability of the complete vRNAP complex.

The Initiation Factor VETF is Anchored to Complete vRNAP

The Vaccinia initiation factor VETF is known to bind promoter DNA up- and downstream of the TSS during initiation of early transcription (Broyles et al., 1991). In the complete vRNAP structure, observed was a central domain of the large VETF subunit (VETF-1³⁶⁵⁻⁴³⁶). This domain has a novel fold that is stabilized by three disulfide bonds and provides the connection between tRNA^{Gln}, the TPase module of VTF/CE and the Rpo18 stalk of the vRNAP core enzyme (FIGS. 23A and 23D). Although only this domain of the 710 amino acid VETF-1 peptide chain is visible in the density, it is likely that the entire heterodimeric protein is anchored to the complex by this means, as VETF-1 and VETF-s were detected in stoichiometric amounts in the sucrose gradient peak fraction (FIG. 17B). Consistent with this, VETF has been described as a stable heterodimer of VETF-1 and VETF-s (Broyles and Moss, 1988). It is likely that during promoter recognition there are major rearrangements in the complete vRNAP that lead to a positioning of mobile VETF regions onto the promoter DNA.

### DISCUSSION

Here is presented a purification procedure for endogenous Vaccinia vRNAP complexes from infected cells and report the first structures of core and complete vRNAP complexes. A comparison to cellular enzymes, in particular eukaryotic Pol II, confirms the common evolutionary origin of multisubunit RNA polymerases and suggests functions of various vRNAP subunits during transcription. Whereas the two large subunits and the active center cleft are generally conserved, peripheral domains, subunits and factors display virus-specific features.

In particular, the viral factor Rap94 associates with vRNAP and contains a central region that resembles the Pol II initiation factor TFIIB and is thus likely involved in transcription initiation. Further, the subunit Rpo30 distantly resembles the Pol II elongation factor TFIIS and likely confers RNA cleavage activity to vRNAP. Such nucleolytic activity appears conserved among multisubunit RNA polymerases and allows for rescue of the transcription machinery in case of backtracking or misincorporation (Fish and Kane, 2002). Whereas the protein that facilitates transcript cleavage is stably associated with Pol I and Pol III (Engel et al., 2013; Fernandez-Tornero et al., 2013; Hoffmann et al., 2015; Neyer et al., 2016), Pol II requires the auxiliary factor TFIIS (Kettenberger et al., 2003). A similar function is fulfilled by the transcript cleavage factors GreA and GreB in bacterial transcription (Borukhov et al., 1993; Opalka et al., 2003; Polyakov et al., 1998; Stebbins et al., 1995). Rpo30 also contains a C-terminal tail that is specific to poxviridae and not found in other large DNA viruses (Mirzakhanyan and Gershon, 2017). Phosphorylation of this tail region occurs in packaged virions (Ngo et al., 2016) and it can occupy the vRNAP active site, raising the possibility that this is a regulatory modification. A comparable observation has been made in the apo form of Pol I, in which a peptide region of the largest subunit occupies the active center cleft (Engel et al., 2013; Fernández-Tornero et al., 2013).

A striking feature of vRNAP is the C-terminal tail located on the largest subunit Rpo147. Whereas this tail is flexible in the core vRNAP complex, it binds to the capping enzyme in the complete vRNAP structure. Although structurally not related, the vRNAP C-tail may thus resemble the Pol II CTD with respect to its function in capping enzyme recruitment, although the Pol II CTD more generally acts as an integration hub for transcription-coupled processes (Harlen and Churchman, 2017; Jasnovidova and Stefl, 2013). The CTD recruits various factors during different phases of transcription in a phosphorylation-dependent manner (Buratowski, 2009; Hsin and Manley, 2012) and is also involved in recruitment of the capping enzyme (Cho et al., 1997; Fabrega et al., 2003; McCracken et al., 1997; Noe Gonzalez et al., 2018). In the accompanying Example, it is shown that the Rpo147 C-tail acts as a tether and alters structure upon rearrangements in the complete vRNAP complex that accompany the formation of an active co-transcriptional capping complex (Hillen et al., this issue of Cell).

The additional factors observed in the complete vRNAP structure are unique to the viral machinery. Rap94 acts as an integral building block of the complete vRNAP, as it bridges the interaction between the polymerase and the associated factors. Consistent with this, a loss of this factor leads to generation of virions that lack vRNAP (Zhang et al., 1994). Rap94 binds NPH-I and locks VTF/CE away from the vRNAP core. The structural similarity and location of the Rap94 central region to TFIIB hint at a functional role during transcription initiation. Consistent with this, Rap94 domain 2 occupies a position that resembles the location of the initiation factor TFIIE in the Pol II pre-initiation complex (Plaschka et al., 2016), and the Rap94 CTD is found at a location that is congruent with that of TFIIF in Pol II initiation complexes (He et al., 2016; Plaschka et al., 2016). Based on its biochemical composition and activity it is likely that the complete vRNAP complex represents a unit that is packaged into viral progenies and used for early viral transcription upon virus entry into a host cell.

Our structures also rationalize known functional data. Antibodies directed against an epitope within the CEC of Rap94 inhibit the formation of the pre-initiation complex (PIC) in vitro (Mohamed et al., 2002), underlining the importance of Rap94 for transcription initiation. Likewise, mutations and deletions within the NPH-I portion of the CEC inhibit termination without affecting its ATPase activity (Mohamed and Niles, 2000; Piacente et al., 2003). For early transcription termination, a sequence motif in the transcribed mRNA triggers the ATPase activity of the ssDNA helicase NPH-I (Broyles, 2003). It has previously been demonstrated that both Rap94 and VTF/CE are involved in the recognition of the termination motif, which may pause the elongating polymerase (Christen et al., 2008; Luo et al., 1995; Tate and Gollnick, 2015). NPH-I may then cause transcript extrusion from the active site by a 5′ to 3′ translocase activity on the non-template strand (Hindman and Gollnick, 2016; Tate and Gollnick, 2011). Provided the observed location of the CEC near the putative RNA exit tunnel is relevant for a termination intermediate, CEC may be involved in the recognition of the termination signal. Finally, the finding that NPH-I structurally resembles chromatin remodeling ATPases supports the forward translocation model of Vaccinia transcription termination.

Also identified were the homodimeric viral core protein E11 as a stoichiometric component of the complete vRNAP. The structure suggests that E11 is a major contributor to the stability of the complete vRNAP. E11 is a late viral product and two temperature-sensitive mutants have been previously identified to map to its gene (Kato et al., 2008; Wang and Shuman, 1996). One of these, G66R, does not affect the virus morphogenesis but rather leads to the formation of non-infectious viral particles under non-permissive conditions (Wang and Shuman, 1996). According to the crystal structure of E11, this G66R mutant maps to a tight beta-hairpin and is likely to be a structural mutant. Of note, temperature sensitive mutations in VETF-s and Rap94 have been reported to lead to a defect in protein packaging into mature virions (Kane and Shuman, 1992; Li et al., 1994). These findings are consistent with the idea that the complete vRNAP is the unit that is incorporated into viral progenies and initiates early transcription immediately after virus internalization during the infection cycle.

The incorporation of an uncharged host tRNA^{Glu} molecule into a transcription complex is so far unprecedented. The tRNA^{Glu} forms an integral part of the complete vRNAP particle, and a presumed loss of tRNA^{Gln} is therefore likely to destabilize the complete vRNAP complex. These observations suggest that it might be part of a regulatory mechanism to synchronize the Vaccinia replication cycle to the metabolic status of the host cell. It is interesting in this regard to note that viral replication critically depends on the amino acid glutamine as the primary energy source (Fontaine et al., 2014). It is hence a possibility that the complete vRNAP forms in the late phase of viral infection when glutamine becomes limiting and uncharged tRNA^{Glu} accumulates.

Vaccinia virus transcription serves as a paradigm for the molecular biology of nucleo-cytoplasmic large DNA viruses, which include poxviruses and the African Swine Fever Virus. Unlike most other viruses which rely on the host transcription machinery, they utilize a virus-encoded multisubunit RNA polymerase, which contains a conserved core in different virus taxa (Koonin and Yutin, 2001; Mirzakhanyan and Gershon, 2017). The vRNAP structures presented here provide the first structural insight into the transcription machinery of poxviridiae. This provides a framework for future studies aimed at a mechanistic characterization of the viral transcription cycle. In particular, snapshots of vRNAP initiation, elongation and termination will shed light on the transitions that occur during these processes and decipher the mechanisms by which the virus-specific factors mediate transcription. As a first step in this direction, provided are structures of transcribing and co-transcriptional capping complexes of Vaccinia vRNAP in the accompanying paper (Hillen et al., submitted in parallel).

Experimental Model and Subject Details

African green monkey kidney fibroblasts (CV-1) were purchased from the American Type Culture Collection (ATCC) and cultured in DMEM (Gibco) supplemented with 10% Fetal Calf Serum (FCS, Gibco) and 1% Penicillin/Streptomycin solution (Gibco). Human HeLa S3 cells were cultured in a 37° C. incubator equilibrated with 5% CO₂ and 95% humidified atmosphere. The cells were cultured in DMEM (Gibco) supplemented with 10% FCS and 1% Penicillin/Streptomycin.

Method Details

Generation of Recombinant Vaccinia Virus GLV-1h439

GLV-1h439 was derived from GLV-1h68 with a HA-tag and FLAG-tag inserted at the end of A24R gene (encoding vRNAP subunit Rpo132). For insertion of the HA/FLAG-doubletag, an A24R transfer vector was constructed. DNA fragments (termed A and B), flanking about 500 bps of each side of the insertion site of the A24R gene were first amplified via PCR with primers A24R-5/A23R-tag3 (product A) and A25Ltag-5/A25L-3 (product B). A second round of PCR linked A and B fragments into product C with primers A24R-5 and A25L-3. The PCR product C was cloned into the pCR-Blunt II-TOPO vector using Zero Blunt TOPO PCR cloning Kit (Invitrogen). The resulting construct pCRII-A24Rtag4 was sequence confirmed. A p7.5E-gpt cDNA fragment (E. coli xanthine-guanine phosphoribosyltransferase gene under the control of vaccinia 7.5 early promoter), released by Xba I and Pst I restriction digest from the TK transfer vector, was then subcloned into pCRII-A24Rtag4. The gpt selection-expression cassette was located outside the Vaccinia virus DNA that directs homologous recombination into the virus genome, allowing for transient dominant selection of vaccinia recombinants (Falkner and Moss, 1990). The final construct A24Rtag-gpt2 was sequence confirmed, and used to make recombinant virus GLV-1h439, with GLV-1h68 as the parental virus.

Viral Replication Analysis

Replication of recombinant GLV-1h439 and GLV-1h68 was performed using a standard plaque assay (Cotter et al., 2017). HeLa S3 cells were grown in 24-well plates and infected with virus at a multiplicity of infection (MOI) of 1. After incubation for 1 h at 37° C., medium was replaced by fresh growth medium and samples were collected 2, 24, 48 and 72 h post viral infection (hpi). After three freeze-thaw cycles, lysates were titrated by plaque assay on CV-1 cells. The assay was performed in triplicate and all samples were measured in duplicates.

vRNAP Purification

For purification of vRNAP from infected cells, Hela S3 cells were grown in 15-cm plates up to 80-90% of confluence. The cells were infected with purified GLV-1h439 with a MOI of 1.2. After 24 h the cells were pelleted and resuspended in lysis buffer (50 mM HEPES, pH 7.5, 150 mM NaCl, 1.5 mM MgCl2, 0.5% [v/v] NP-40, 1 mM DTT, and complete EDTA-free protease inhibitor cocktail [Sigma-Aldrich]). For vRNAP purification, the extract was incubated for 3 h at 4° C. with 200 μl anti-FLAG Agarose (Sigma). Beads were washed four times with buffer containing 50 mM HEPES, pH 7.5, 150 mM NaCl, 1.5 mM MgCl2, 0.1% [v/v] NP-40 and 1 mM DTT and equilibrated with elution buffer (50 mM HEPES, pH 7.5, 150 mM NaCl, 1.5 mM MgCl2 and 1 mM DTT). The bead-bound proteins were eluted with 3×FLAG peptide, resolved in 12% Bis-Tris gels and visualized by silver staining. For purification of native vRNAP, the eluate of the anti-FLAG column was concentrated to 1 mg/ml and layered on top of a 10%-30% sucrose gradient and centrifuged for 16 h and 35.000 rpm at 4° C. in a Beckman 60Ti swing-out rotor. Gradient fractions were fractionated manually, separated by SDS-PAGE and proteins visualized by silver staining.

Initiation Assay

Plasmid pSB24, containing a G-less cassette downstream of a synthetic vaccinia virus early promoter was generously provided by Dr. Steven Broyles (Purdue University). Construction of the pSB24 vector with Vaccinia virus early termination signal was described in (Luo et al., 1991). Briefly, by standard genetic manipulation, the sequence from BamHI site to HindIII site of the pSB24 was replaced with the duplex oligonucleotides. The insert sequences include three tandem copies of Vaccinia early termination signal. A typical in vitro transcription had a volume of a 50 μl and contained 40 mM Tris-HCl, pH 7.9, 1 mM DTT, 2 mM spermidine, 6 mM MgCl2, 1 mM ATP, 1 mM CTP, 1 mM GTP, 0.1 mM UTP, 20 μCi α[³²P]-UTP [6000 Ci/mmol], 80 μM SAM, 400 ng of NdeI-linearised pSB24 template as well as purified core or complete vRNAP (Luo et al., 1991). The reaction was incubated at 30° C. for the indicated time points before RNA was extracted and precipitated with isopropanol. Transcripts were analysed by denaturing gel electrophoresis and visualised by autoradiography.

Mass Spectrometry Analysis

For protein identification by in-gel digestion each gel lane was cut into 15 slices. The gel bands were destained with 30% acetonitrile in 0.1 M NH₄HCO₃ (pH 8.0), shrunk with 100% acetonitrile, and dried in a vacuum concentrator (Concentrator 5301, Eppendorf, Germany). Digests were performed with 0.1 μg trypsin per gel band overnight at 37° C. in 0.1 M NH₄HCO₃ (pH 8.0). After removing the supernatant, peptides were extracted from the gel slices with 5% formic acid, and extracted peptides were pooled with the supernatant. Nano LC-MS/MS analyses were performed on an Orbitrap Fusion (Thermo Scientific) equipped with a PicoView Ion Source (New Objective) and coupled to an EASY-nLC 1000 (Thermo Scientific). Peptides were loaded on capillary columns (PicoFrit, 30 cm×150 μm ID, New Objective) self-packed with ReproSil-Pur 120 C18-AQ, 1.9 μm (Dr. Maisch) and separated with a 30-minute linear gradient from 3% to 30% acetonitrile and 0.1% formic acid and a flow rate of 500 nl/min. Both MS and MS/MS scans were acquired in the Orbitrap analyzer with a resolution of 60,000 for MS scans and 15,000 for MS/MS scans. HCD fragmentation with 35% normalized collision energy was applied. A Top Speed data-dependent MS/MS method with a fixed cycle time of 3 seconds was used. Dynamic exclusion was applied with a repeat count of 1 and an exclusion duration of 30 seconds; singly charged precursors were excluded from selection. Minimum signal threshold for precursor selection was set to 50,000. Predictive AGC was used with AGC a target value of 2e5 for MS scans and 5e4 for MS/MS scans. EASY-IC was used for internal calibration. Data analysis was performed against UniProt Vaccinia Virus database with PEAKS 8.5 software (Bioinformatics Solution Inc.) with the following parameters: parent mass tolerance: 8 ppm, fragment mass tolerance: 0.02 Da, enzyme: trypsin, variable modifications: oxidation (M), pyro-glutamate (N-term. Q), phosphorylation (STY), carbamidomethylation (C). Results were filtered to 1% PSM-FDR by target-decoy approach.

Cross-Linking Mass Spectrometry (XLMS)

Protein cross-linking of purified complexes and subsequent mass spectrometry was performed as described previously (Vos et al., 2018). Briefly, samples were crosslinked with BS3 (ThermoFisherScientific) and incubated for 30 min at 30° C. The reaction was quenched by adding 100 mM Tris-HCl pH 7.5 and 20 mM ammonium bicarbonate (final concentrations) and incubation for 15 min at 30° C. Proteins were precipitated with 300 mM sodium acetate pH 5.2 and four volumes of acetone overnight at −20° C. The protein was pelleted by centrifugation, briefly dried, and resuspended in 4 M urea and 50 mM ammonium bicarbonate. Crosslinked proteins were reduced with DTT and alkylated (Vos et al., 2016). After dilution to 1 M urea with 50 mM ammonium bicarbonate (pH 8.0), the crosslinked protein complex was digested with trypsin in a 1:50 enzyme-to-protein ratio at 37° C. overnight. Peptides were acidified with trifluoroacetic acid (TFA) to a final concentration of 0.5% (v/v), desalted on MicroSpin columns (Harvard Apparatus) following manufacturer's instructions and vacuum-dried. Dried peptides were dissolved in 50 μl 30% acetonitrile/0.1% TFA and peptide size exclusion (pSEC, Superdex Peptide 3.2/300 column on an ÄKTAmicro system, GE Healthcare) was performed to enrich for crosslinked peptides at a flow rate of 50 μl min−1. Fractions of 50 μl were collected. Fractions containing the crosslinked peptides (1-1.7 ml) were vacuum-dried and dissolved in 2% acetonitrile/0.05% TFA (v/v) for analysis by LC—MS/MS.

Crosslinked peptides were analysed as technical duplicates on an Orbitrap Fusion or Orbitrap Fusion Lumos Tribrid Mass Spectrometer (Thermo FisherScientific), coupled to a Dionex UltiMate 3000 UHPLC system (ThermoFisherScientific) equipped with an in-house-packed C18 column (ReproSil-Pur 120 C18-AQ, 1.9 μm pore size, 75 μm inner diameter, 30 cm length, Dr. Maisch GmbH). Samples were separated applying the following 58 min gradient: mobile phase A consisted of 0.1% formic acid (v/v), mobile phase B of 80% acetonitrile/0.08% formic acid (v/v). The gradient started with 5% B, increasing to 8% B on Fusion and 15% on Fusion Lumos, within 3 min, followed by 8-42% B and 15-46% B within 43 min accordingly, then keeping B constant at 90% for 6 min. After each gradient the column was again equilibrated to 5% B for 6 min. The flow rate was set to 300 nl min-1. MS1 spectra were acquired with a resolution of 120,000 in the Orbitrap covering a mass range of 380-1580 m/z. Injection time was set to 60 ms and automatic gain control target to 5×105. Dynamic exclusion covered 10 s. Only precursors with a charge state of 3-8 were included. MS2 spectra were recorded with a resolution of 30,000 in the Orbitrap, injection time was set to 128 ms, automatic gain control target to 5×104 and the isolation window to 1.6 m/z. Fragmentation was enforced by higher-energy collisional dissociation at 30%.

Raw files were converted to mgf format using ProteomeDiscoverer 1.4 (Thermo Scientific, signal-to-noise ratio 1.5, 1,000-10,000 Da precursor mass). For identification of crosslinked peptides, files were analysed by pLink (v. 1.23), pFind group (Yang et al., 2012) using BS3 as crosslinker and trypsin as digestion enzyme with maximal two missed cleavage sites. Carbamidomethylation of cysteines was set as a fixed modification, oxidation of methionines as a variable modification. Searches were conducted in combinatorial mode with a precursor mass tolerance of 5 Da and a fragment ion mass tolerance of 20 p.p.m. The used database contained all proteins within the complex. The false discovery rate was set to 0.01. Results were filtered by applying a precursor mass accuracy of ±10 p.p.m. Spectra of both technical duplicates were combined and evaluated manually.

RNAseq Analysis

Libraries were generated from the isolated RNA fraction following the Ion Torrent™ Ion Total RNA-seq kit v2 (Thermo Fisher; Art. No. 4475936) protocol with the following modifications. Before the libraries were generated 40 ng of the gel-purified RNA was digested with 10U of RNAse T1 (Thermos Fisher; Art. No. EN0541) for 1 minute at room temperature. After PCI extraction and ethanol precipitation, the RNA was pre-treated with 5 U Antarctic phosphatase (New England Biolabs; Art. No. M0289) for 30 minutes at 37° C. After heat inactivation at 65° C., the RNA was phosphorylated by 20 U T4 polynucleotide kinase (New England Biolabs; Art. No. M0201) for 60 minutes at 37° C. Adapter ligation was carried out for 16 hours at 16° C. followed by an incubation of 10 minutes at 50° C. Reverse transcription (RT) was performed employing SuperScript™ III with incubations at 42° C., 50° C. and 55° C. for 45, 15 and 10 minutes, respectively. The RT reactions were purified and the cDNA was amplified by Platinum PCR SuperMix High Fidelity. Resulting libraries were sequenced using a Ion Proton (Ion Torrent™) with high-Q.

Structure Determination of Core vRNAP

Following sucrose gradient purification, fraction 11 (FIG. 17B) was diluted 1:50 and concentrated in a Vivaspin concentrator to a concentration of roughly 50 μg/ml to remove the sucrose. For cryo-EM analysis the sample was centrifuged for 2h at 21,000 g and diluted 1:1 in a buffer containing 20 mM HEPES, pH 7.5, 200 mM (NH₄)₂SO₄, 1 mM MgCl₂ and 5 mM 2-mercaptoethanol. 4 μl of sample were applied to glow discharged UltrAu 2/2 (Quantifoil) grids at 4° C. and 95% humidity in a Vitrobot (FEI Company), blotted for 8.5 s at blot force 14 and plunge-frozen in liquid ethane. Cryo-EM data was collected on a Titan Krios G2 electron microscope (FEI Company) operated at 300 kV with a K2 direct electron detection device operated in counting mode (Gatan) and an energy filter (Gatan) set to a slit width of 15 eV. Movie stacks of 39 frames were acquired with a total dose of 55 e⁻/Å² in counting mode at a nominal magnification of 165,000×, corresponding to a calibrated pixel size of 0.81 Å/pixel. Dose weighting and motion correction was performed using MotionCor2 (Zheng et al., 2017). Per-micrograph contrast-transfer function (CTF) estimation was done using Gctf (Zhang, 2016), as implemented in Relion (Scheres, 2012). A subset of 4,065 particles was manually picked from the micrographs and used for reference-free 2D classification in Relion and the resulting class averages were used to generate reference projections. These were then used as templates for automated particle picking using Gautomatch (http://www.mrc-lmb.cam.ac.uk/kzhang/).

A total of 479,618 particles were extracted with a box size of 300 pixels in Relion and subjected to reference-free 2D classification followed by initial global 3D refinement using the B. taurus Pol II elongation complex structure as reference (EMD 3218) (Bernecky et al., 2016), which yielded a reconstruction at 3.1 Å overall resolution (FIG. 25). Further 3D classification revealed two distinct states of vRNAP corresponding to ‘open’ and ‘closed’ state clefts, similar to the motion observed previously for Pol II (Cramer et al., 2000; 2001). As the two reconstructions did not show any further differences and the closed state class contained more particles, this class was used for further refinement. Per-particle CTF and motion correction was performed on this particle subset using Warp (Tegunov and Cramer, 2018) and CTF and beam tilt refinement was additionally performed using Relion. The resulting final reconstruction from 3D refinement in Relion achieved an overall resolution of 2.8 Å after post-processing with a sharpening B-factor of −79 Å². This cryo-EM density was of excellent quality, with clear sidechain densities for the majority of the complex and occasional density for bound ions. However, modelling ions or waters was refrained from, with the exception of the catalytic metal ion A as its location and identity can be inferred from previous crystallographic studies as well as the structural zink ions which are each complexed by four cysteine or histidine residues. In addition to the well-resolved core, the cryo-EM map showed fragmented densities on either side of the vRNAP cleft which were not of sufficient quality for model building. To improve these regions, soft masks encompassing them were cut out from the global reconstruction that was previously low-pass filtered to 10 Å. Focused 3D classification using these masks and the particle subset used in the global refinement was then used to identify particle subpopulations with strong occupancy in the desired region. These particle subpopulations were then subjected to focused 3D refinement, which was initially run without a reference mask until the refinement converged to local searches, from where on the respective mask was provided for alignment of particles within the masked region. Post-processing of these maps was performed in Relion using the same soft masks also used in focused classification and refinement. This approach yielded improved densities for the previously poorly resolved regions.

The initial model of core vRNAP was constructed by docking homology models of Rpo147 and Rpo132 generated by Swissmodel (Biasini et al., 2014) into the cryoEM density, followed by manual rebuilding of all residues in Coot (Emsley et al., 2010). Subunits Rpo35, Rpo22, Rpo19, Rpo18 and Rpo7 were built de novo in Coot. The density for the most distal strands of Rpo18 was weak and improved only moderately upon focused classification and refinement, thus indicating potential mobility. Subunit Rpo30 was built de novo in the improved map obtained by focused refinement for its binding region. Cross-linking coupled to mass-spectrometry indicated that the initially fragmented densities remaining on either side of the cleft represent Rap94 (FIG. 25H), and these regions could be built de novo after focused classification and refinement in the respective maps. The Rap94 linker regions L2 and L4 could be partially built de novo in the global reconstruction. After fitting of all models, very weak density remained at the back of vRNAP, which corresponds to the B-homology domain of Rap94. Extensive focused classification and refinements efforts on this region yielded improved maps around the B-ribbon and B-cyclin domains, but these were not of sufficient quality for reliable model building and thus omitted these parts were from the core vRNAP model. In total, the structure contains models for Rpo147 (UniProt B9U1I2; res. 2-207; 217-1268), Rpo132 (UniProt B9U1Q1; res. 8-122; 126-418; 422-448; 458-789; 797-825; 841-1162), Rpo35 (UniProt B9U1R2; res. 3-305), Rpo22 (UniProt B9U1I0; res. 1-184); Rpo19 (UniProt B9U1M4; res. 61-164), Rpo18 (UniProt B9U1K4; res. 2-108; 136-159), Rpo7 (UniProt B9U1G3; res. 2-62), Rpo30 (UniProt B9U1D1; res. 23-62; 67-151) and Rap94 (UniProt B9U1I7; res. 106-134; 160-316; 588-619; 627-650; 655-795). The structure was refined using phenix.real_space_refine (Adams et al., 2010) against a composite map generated from the global refinement map and the focused refinement map using phenix.combine_focused_maps by weighting the individual parts according to their cross-correlation with the model. To validate this approach, the model was similarly refined against the locally sharpened density obtained during the Relion local resolution estimation, which yielded comparable final results. The final structure displays excellent stereochemistry, as verified by Molprobity (Chen et al., 2010).

Figures were created with PyMol (Schrodinger, LLC, 2015) and UCSF Chimera (Pettersen et al., 2004). Angular distribution plots were created using a tool distributed with Warp (Tegunov and Cramer, 2018). Sequence identity scores were calculated using Ident and Sim (website bioinformatics.org/sms2/ident_sim.html) (Stothard, 2000) with the structure-based alignments as input.

Structure Determination of Complete vRNAP

Sample were prepared as for the core vRNAP. For cryo-EM data collection, R 1.2/1.3 holey carbon grids (Quantifoil) were glow discharged for 90 s (Plasma Cleaner model PDC-002. Harrick Plasma Ithaca, N.Y./USA) at medium power and 3.5 μl of C2 sample was applied inside a Vitrobot Mark IV (FEI) at 4° C. and 100% relative humidity. The grids were blotted for 3 s and with blot force 5 and plunged into liquid ethane. The Cryo-EM datasets were collected with a Thermo-Fisher Titan Krios G3 and a Falcon III camera (Thermo-Fischer). Data was acquired with EPU at 300 keV and a primary magnification of 75,000 (calibrated pixel size 1.0635 Å) in movie-mode with 25 fractions per movie and integrating the electron-signal. The total exposure was 50 e/Å² over an exposure time of 4.5 s with 2 exposures per hole.

Dose-weighted, motion-corrected sums of the micrograph movies were calculated with Motioncorr2 (Zheng et al., 2017). The contrast-transfer function of each micrograph was fitted with CTFFind4 (Rohou and Grigorieff, 2015). An initial set of 1,500 particles was selected manually and subjected to a 2D-Classification in Relion3-beta (Zivanov et al., 2018). 12 reasonable class averages were selected as templates for subsequent automated particle picking within Relion and 256,452 particles were picked from 2,224 micrographs. The dataset was then cleaned up by four cycles of 2D classification and particle sorting followed by manual selection of classes based on the appearance of their class averages resulting in a final dataset of 190,000 good particles. A subset of 20,000 particles was used to generate an initial model. An initial 3D classification with Relion yielded two major classes which differed obviously in the density for VTF/CE, and were subjected to 3D refinement. The class of the large particle yielded a 3.3 Å reconstruction. A second round of automated particle picking was performed with projections from the reconstruction of the large particle as picking templates and yielded a dataset of 858,702 particles. This dataset was then cleaned up by four cycles of 2D classification and particle sorting followed by manual particle selection resulting in a final dataset of 618,338 good particles. A 3D classification of this dataset yielded only highly similar classes and the reconstruction using the full, unclassified dataset yielded the highest resolution of 2.98 Å. With further per-particle CTF refinement including a per-dataset beam-tilt refinement and per-particle motion-correction (‘polishing’) within Relion3 a reconstruction was obtained with 2.75 Å resolution.

For model building and refinement, the complete vRNAP density was unambiguously docked with the previously built core vRNAP model, the crystallographic models of VTF/CE (PDB ID 4CKB) (Kyrieleis et al., 2014), the E11 homodimer and bacterial tRNA^{Gln} extracted from PDB entry 1GSG. The residual density for VETF-1365-436, NPH-I, and Rap94 was assigned and traced manually within Coot (Emsley et al., 2010) with the guidance of secondary structure predictions from PsiPred (Jones, 1999) and XLMS data. The final model was refined with Phenix.real_space_refine including an ADP refinement step. During refinement secondary structure, mild Ramachandran and reference model restraints from the VTF/CE and E11 crystallographic models were imposed. After a further cycle of manual inspection and automated refinement, water molecules were placed with Coot and a final round of refinement with Phenix.real_space_refine was applied.

X-Ray Structure Determination of E11

Bacterially overexpressed, hexa-histidine tagged E11 protein was bound to Ni-NTA-Agarose, eluted with 200 mM Imidazole and dialysed against TBS. The tag was cleaved with tobacco etch virus protease and a final gel filtration chromatography was performed. Crystals were obtained with the hanging drop vapour diffusion method with reservoir solution containing 20% PEG 4000. For crystallographic phase determination the crystals were derivatized with sodium ethylmercurithiosalicylate and a SAD experiment was performed at beamline MX1/P13 of the PETRA III storage ring of the Deutsches Elektronen-Synchrotron (DESY). Phasing and initial model building were performed with Phenix.autosol. The model was then refined against a native dataset collected at the same beamline with Phenix.refine and completed manually within Coot. After three more cycles of manual corrections and automated refinement including water placement and TLS refinement, the R-factors converged.

### Example 4. Structure of Poxvirus Transcription Pre-Initiation Complex in the Initially Melted State

Multi-subunit DNA-dependent RNA polymerases (RNAPs) catalyze nuclear transcription of eukaryotic genes. While many viruses seize the host transcription machinery to express their genome, poxviruses replicate in the cytoplasm and thus depend on a unique viral RNAP (vRNAP). Here is presented the cryo-EM structure of the vRNAP pre-initiation complex (PIC) from the poxvirus vaccinia, disclosing how the heterodimeric transcription factor VETFUs enables viral transcription initiation. VETF adopts an arc-like shape, spans the polymerase cleft and anchors upstream and downstream promoter elements. Four domains of VETFI cooperate in upstream promoter recognition, enforcement of transcription directionality, and PIC stabilization. A fifth domain adopts a TATA binding protein-like fold that inserts asymmetrically into the DNA major groove, and triggers bending and initial melting of promoter DNA. VETFs, which displays a helicase fold that contacts the downstream promoter, induces a sharp bend in the DNA helix and fosters the initial melting event around the transcription start site. The structure, with the first bilobal TBP-like protein solved thus far, sheds light on the unique mode of poxvirus transcription initiation and provides the basis to assess the evolution of cytoplasmic transcription.

Gene transcription by DNA-dependent RNA polymerases (RNAPs) is the first step in the expression of the genome in all forms of life. Eukaryotic RNAPs are multi-subunit complexes that act in the cell nucleus or in DNA-containing organelles. Most DNA viruses make use of the nuclear transcription machinery of the host to express their genome. A remarkable exception are poxviruses, which cause smallpox in humans and various zoonoses¹⁻³. They replicate exclusively in the cytoplasm of infected cells and thus depend on their own set of transcription and mRNA processing factors. Studies on the prototypic poxvirus vaccinia identified a multi-subunit RNA polymerase (vRNAP) and factors that ensure the production of polyadenylated and m⁷G-capped mRNA⁴⁻⁸. Vaccinia gene expression has been biochemically well characterized, but only recently, cryo-EM gave insight into the structure of vRNAP complexes and their mechanisms of transcription elongation and transcription-coupled capping^{9,10}. These studies confirmed the evolutionary relationship of core vRNAP with the three eukaryotic RNAPs, but also revealed strong idiosyncrasies with regard their interacting factors¹¹⁻¹⁴.

A feature of core vRNAP is its association with five virus-encoded proteins and one host factor: the TFIM¹⁵-related transcription factor Rap94^{16,17}, the viral early transcription factor VETF, a heterodimer of subunits VETFs and VETF1^{7,18,19}, the capping enzyme D1/D12²⁰, the helicase NPH-I²¹, the core protein E11, and cellular tRNA^{Gln}. This unit, termed complete vRNAP, is necessary and sufficient to target the polymerase to early promoters and enable transcription of vaccinia early genes. Early genes are controlled by promoters containing a single, A/T-rich consensus sequence (the critical region, CR)²² located upstream of the transcription start site (TSS, Extended Data FIG. 1a). Here, complete vRNAP was used to reconstitute and purify the early promoter pre-initiation complex (PIC). The cryo-EM reconstruction of the PIC reveals the atomic structure of VETF bound to promoter DNA in the initially melted state and uncovers a thus far unknown mechanism of promoter recognition.

Cryo-EM Structure of the Vaccinia Pre-Initiation Complex

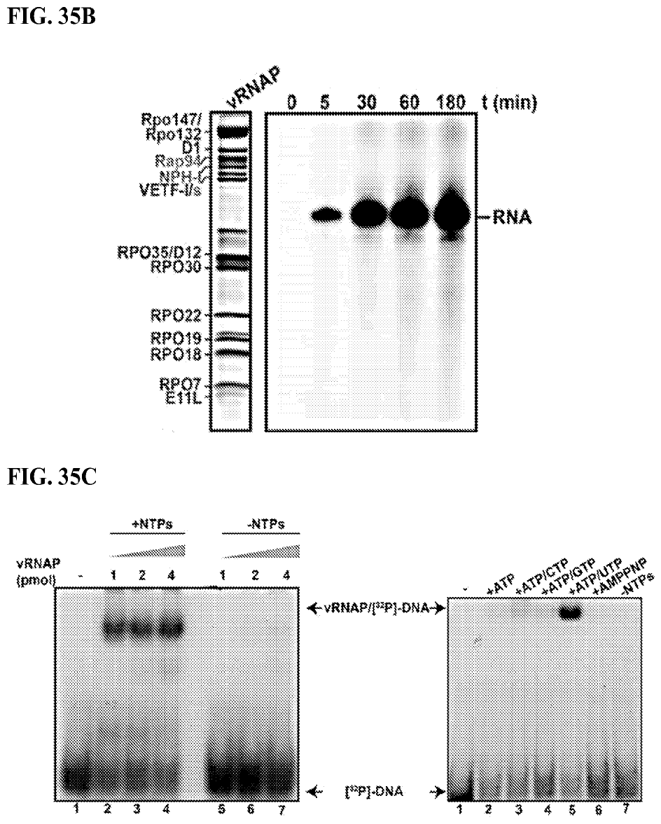

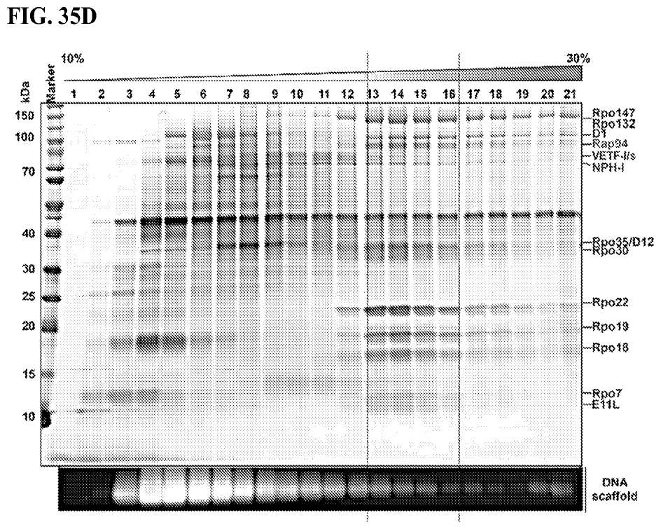

Complete vRNAP was affinity-purified from HeLa cells infected with an engineered vaccinia strain that expresses a FLAG-tagged vRNAP subunit, Rpo132¹⁰. The transcriptionally active complete vRNAP was used to reconstitute a complex with a DNA duplex that mimics the viral early promoter (FIG. 35b-35d). The DNA-bound vRNAP was isolated by gradient centrifugation (FIG. 35e), and three cryo-EM datasets were collected.

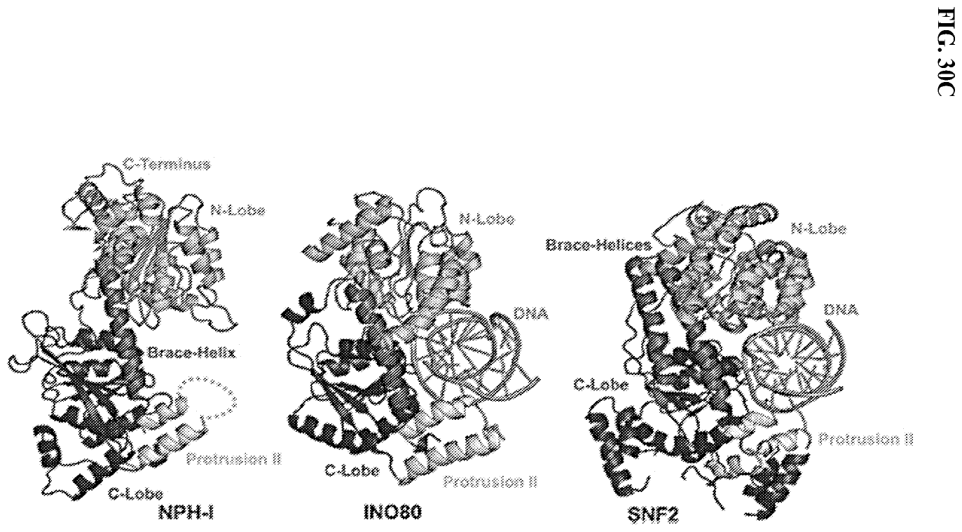

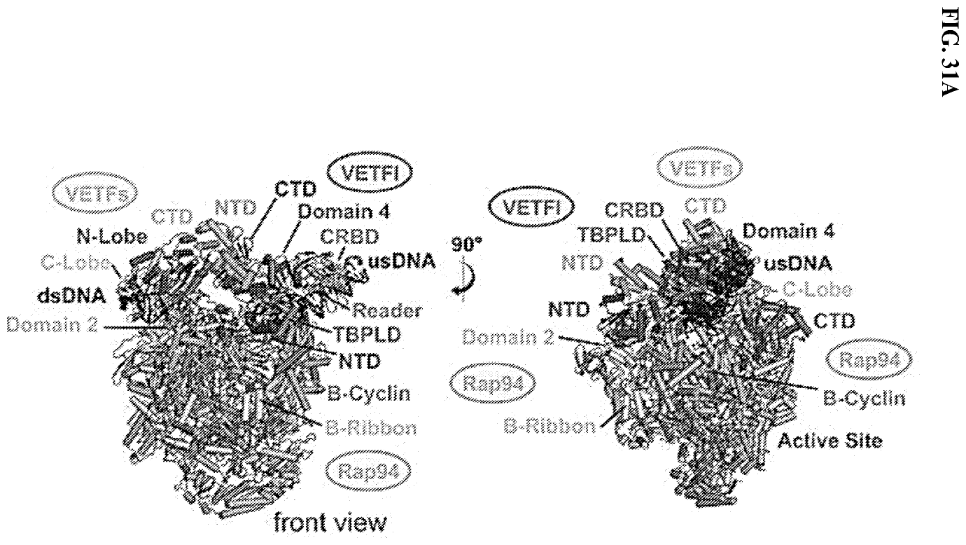

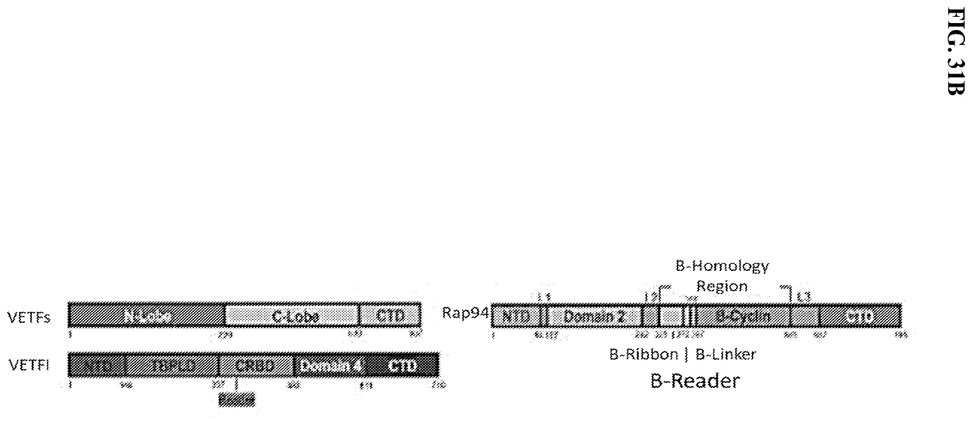

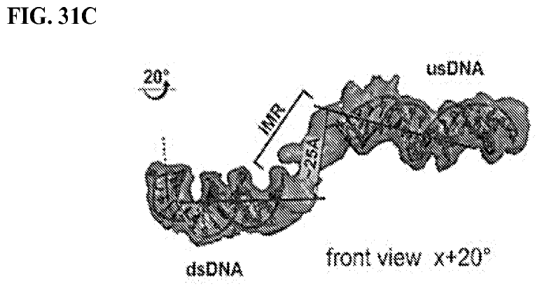

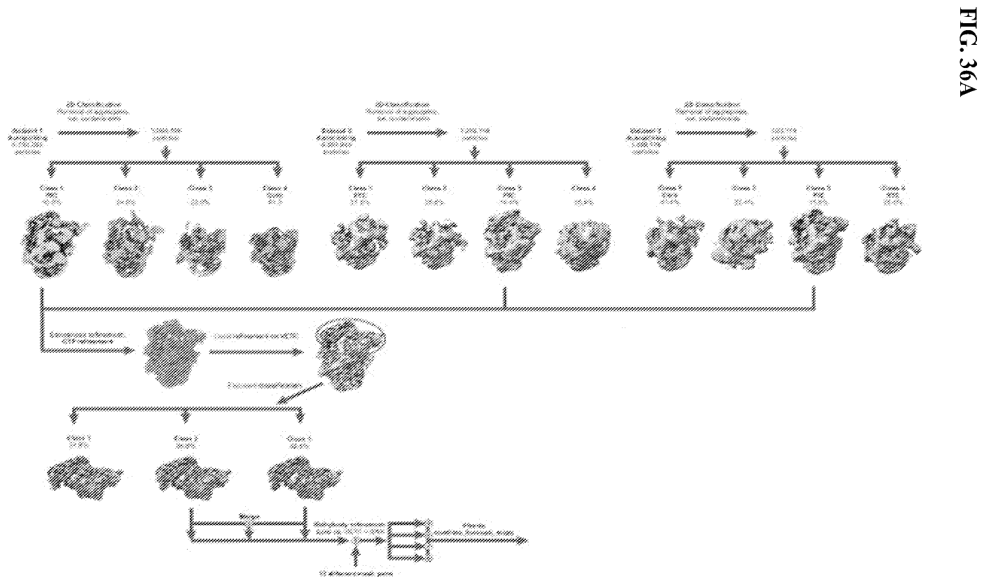

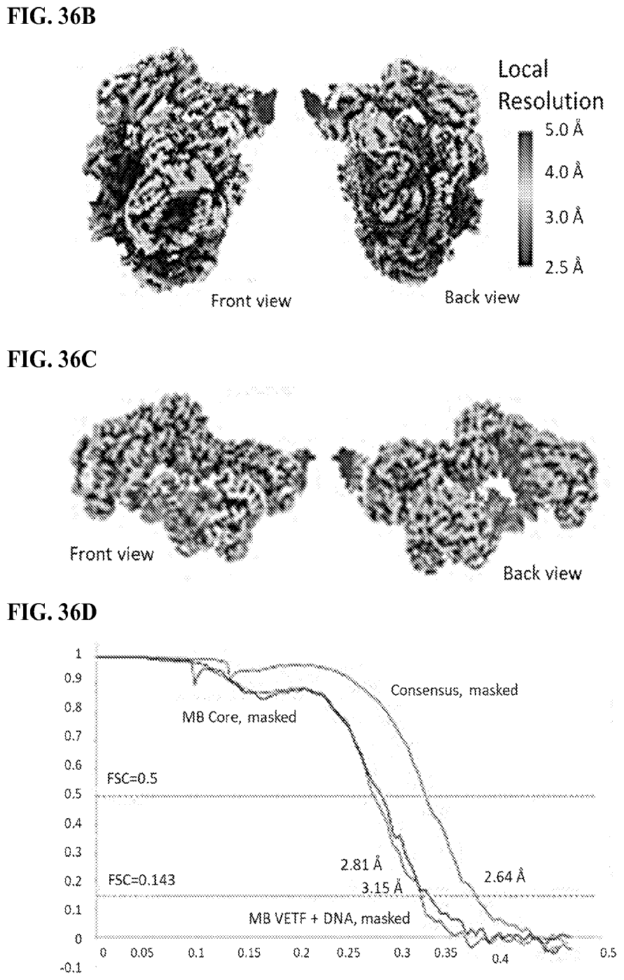

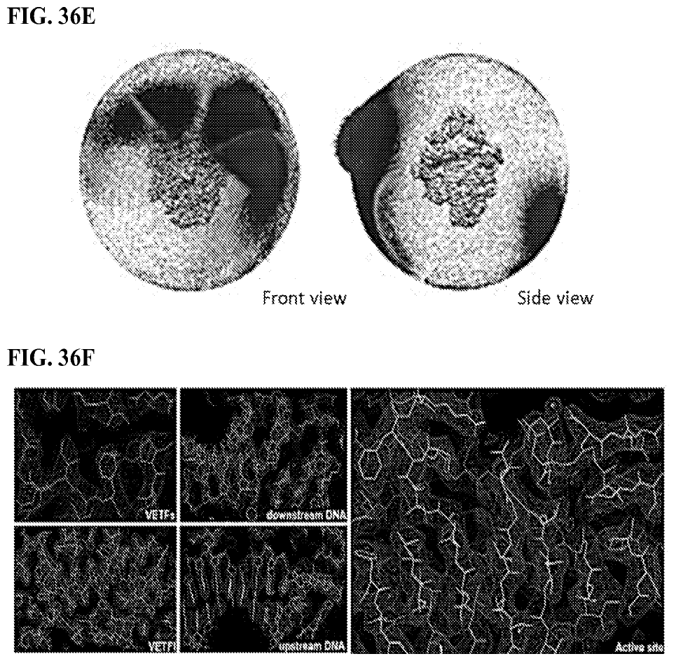

After extensive 3D classification, several distinctive vRNAP particle classes could be separated (FIG. 36a) that represented different transcription stages from the pre-initiation phase to capping (see also accompanying paper). One class represented the bona fide PIC, since it contained the core vRNAP together with initiation factors VETF^{16,23,24} and Rap94, and promoter DNA. The single-particle reconstruction of this class displayed an overall resolution of 3.0 Å with diffuse density for DNA and VETF. Signal subtraction and focused refinement resolved the VETF-DNA subcomplex at a local resolution ranging from 2.9 Å to 4.0 Å (Extended Data FIG. 2b-f, Extended Data Tab. 1). The density was docked with the core vRNAP model, manually adjusted, and the VETF1 and VETFs chains were traced de novo, thus allowing modelling of the entire PIC (FIG. 31a).

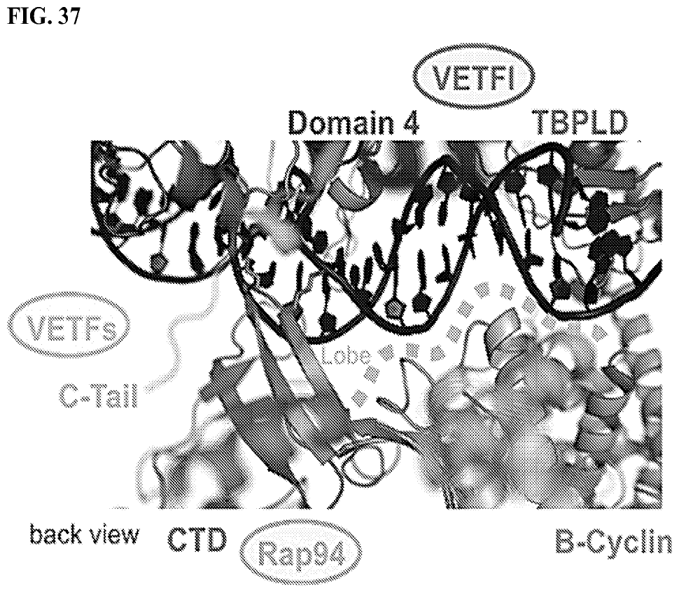

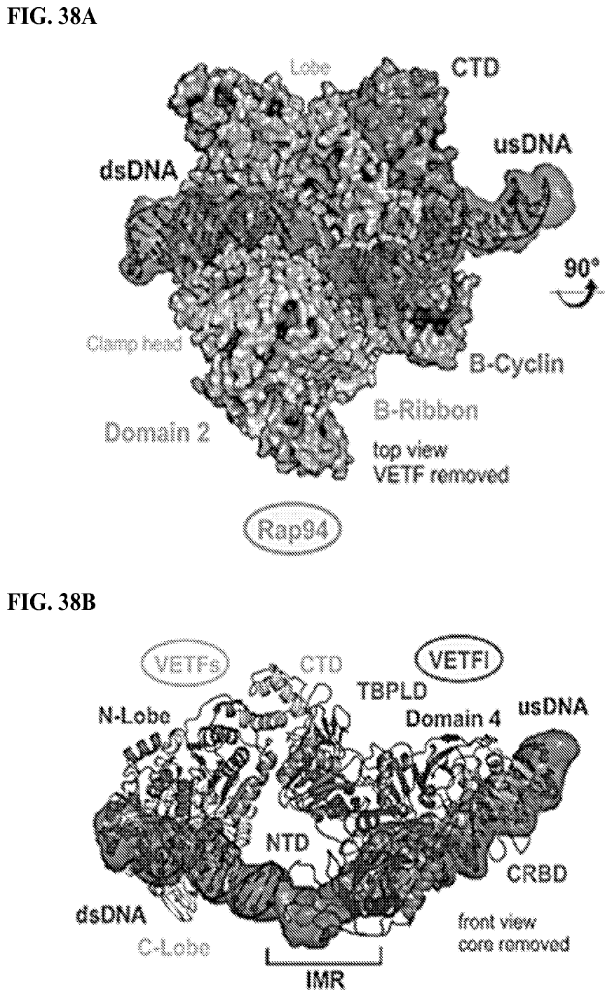

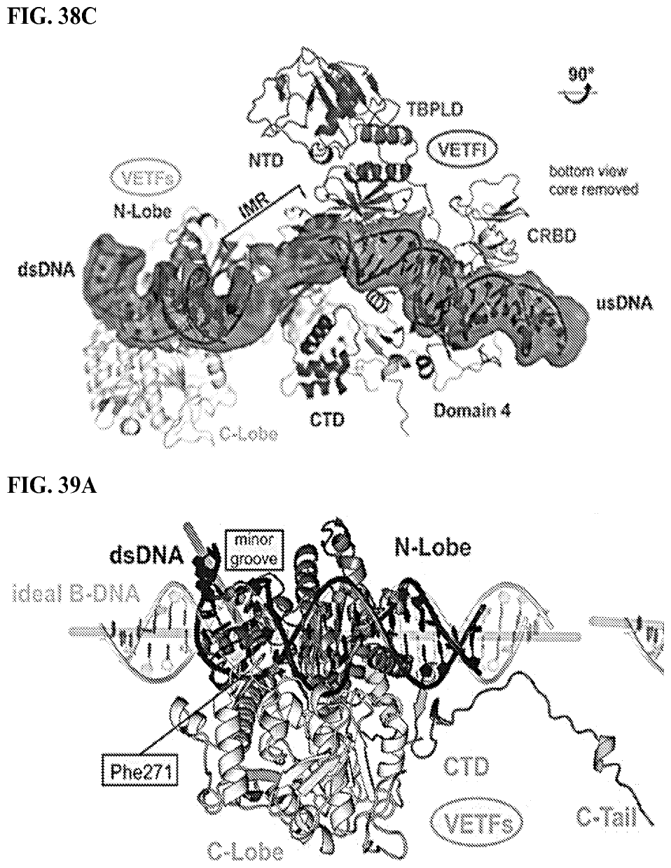

Within the PIC, the promoter is positioned above the polymerase cleft. The upstream DNA contacts the protrusion domain of the polymerase subunit Rpo132, directly adjacent to the C-terminal domain (CTD) of Rap94 (FIG. 31a, 31b and FIG. 37). The downstream promoter region interacts with the vRNAP core through positions on the clamp head (FIG. 31a, 31b, FIG. 38a). The melted promoter region is predominantly disordered but could be visualized with mild Gaussian filtering (FIG. 31c). It localizes centrally above the opening of the cleft forming a second contact zone with the clamp head (FIG. 38a). Both DNA strands appear only minimally separated within the bubble region. The latter joins the adjacent double-helical upstream and downstream sections in a 100° angle accompanied by a 25 Å translational shift of the helix axes (FIG. 31c). The structural data thus indicate that the DNA is in the initially melted state.

Of note, neither the B-homology region, nor other domains of the early transcription factor Rap94 establish DNA contacts (FIG. 31a, 31b). However, on the opposite side of the core vRNAP, VETFs and VETF1 are engaged in extensive DNA contacts in the respective distal upstream and downstream promoter regions. Therefore, and due to the absence of contacts in the initially melted region (IMR), the VETF heterodimer appears to be anchored like a bridge on both, the upstream and downstream region of the promoter (FIG. 31a and FIG. 38b).

Structure of the DNA-Bound VETF Heterodimer

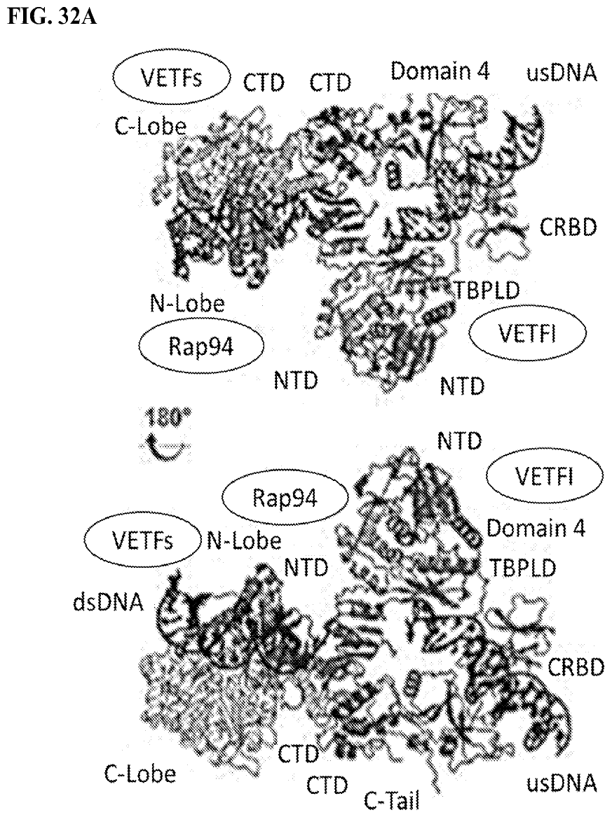

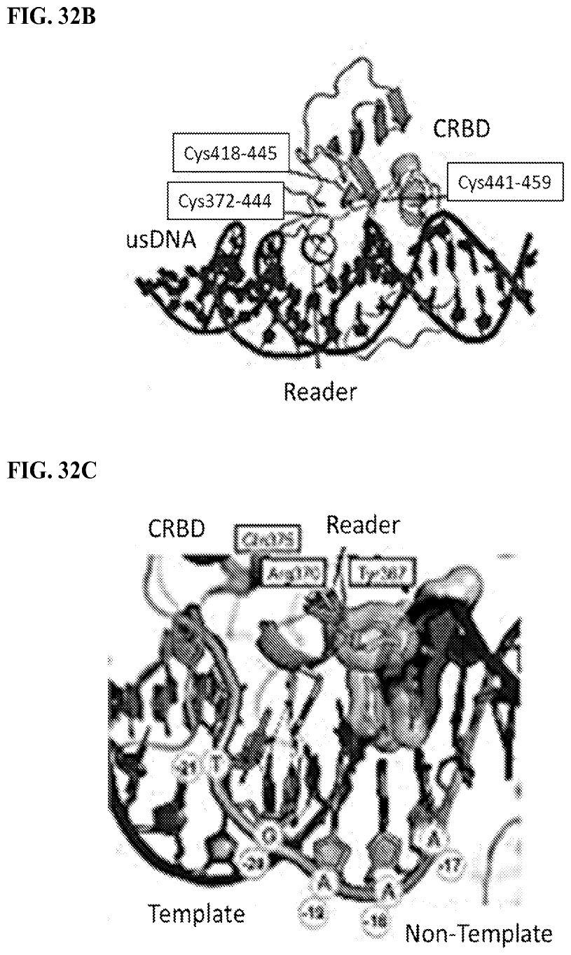

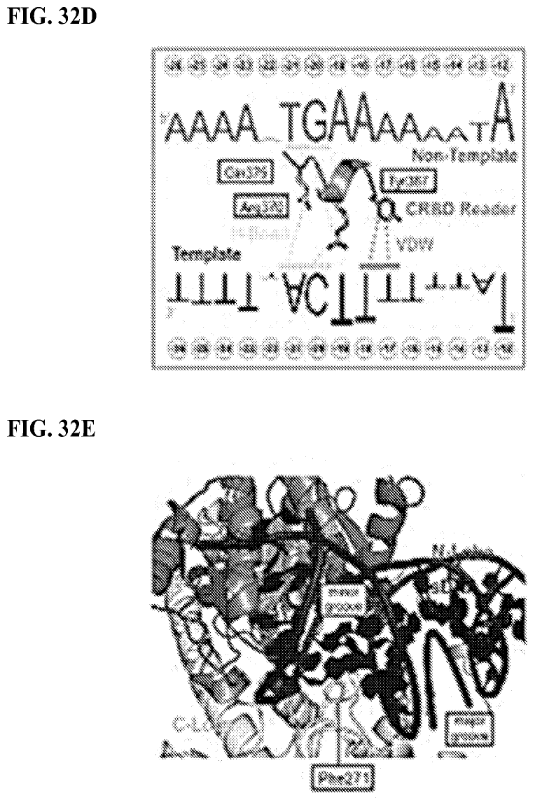

The structure of VETF allowed deciphering of the mechanisms of core vRNAP binding to the early promoter. VETF1 folds into five distinct domains, termed NTD, TBPLD, CRBD, Domain 4 and CTD (FIG. 31b). Despite the absence of any detectable sequence homology, the second domain displays a bi-lobal TATA-box binding protein (TBP) fold, and hence is a TBP-like domain (TBPLD). It is located centrally above the polymerase cleft and, unlike bona fide TBP, contacts the promoter in a sequence-independent manner. Instead, sequence-specific DNA binding is facilitated by the neighboring domain (FIG. 31b), which establishes the upstream promoter contact by recognizing the CR (FIG. 32a, 32b). Based on its fold and binding mode, it constitutes a novel type of double-stranded DNA binding domain, hence termed Critical Region Binding Domain (CRBD). While holding only a limited content of secondary structure elements, it gains structural rigidity through three disulfide bridges that position a 3₁₀-helix ideally for its insertion into the major groove of the DNA (FIG. 32b). The sidechain-to-base contacts of this helix are the major site for sequence-specific readout of the promoter sequence (FIG. 32c, 32d). Only weak bending of the DNA helix axis is introduced in this region (FIG. 32a, 32b).

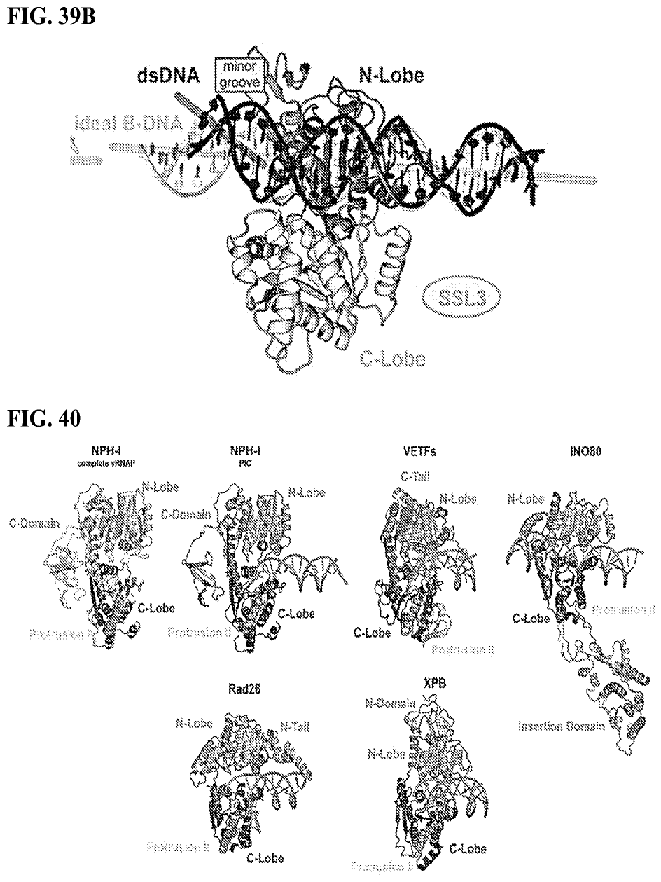

The joint structural context of TBPLD and CRBD establishes specific contacts of VETF-I to the upstream promoter. The latter is anchored on the core vRNAP via the interaction of domain 2 of Rap94 with the NTD of VETF1 (FIG. 31a, FIG. 39). All other domains of VETF-I (NTD, Domain 4 and CTD) contribute to the structural backbone of VETF. Domain 4 and the CTD of VETF1 make up the interface to VETFs (FIG. 32A).

The downstream promoter interacts almost exclusively with VETFs (FIG. 31a, FIG. 32 a, 32e). Only one additional pointed contact to the core vRNAP is established by the clamp head close to the TSS (FIG. 37). Observed was a striking similarity of the first two domains of VETFs with the canonical helicase fold of chromatin remodeling SNF2-type ATPases, of which INO80 is the closest homologue^{11,19}. With the latter, VETFs shares, along with the vRNAP-associated transcription factor NPH-I, an extended brace helix that stably bridges N- and C-lobe of the helicase fold (FIG. 40). The intense DNA interaction of the VETFs helicase module is accompanied by a strong bend of the helix (FIG. 38a). At the point of inflection, Phe271 intercalates via the minor groove, effectively disturbing the planar base-stacking over the range of roughly 3 base pairs on either side of the insertion site (FIG. 32c). Although melting of the two DNA strands at this position is not observed in the vaccinia PIC, this mechanism bears some similarity to the ‘scalpel’ method of strand-separating helicases²⁵.

Positioning on the Promoter and Enforcement of Transcription Directionality

Next it was asked how the DNA contacts established by the CRBD of VETF1 control the initiation process. The 3₁₀-helix of CRBD inserts into the major groove, making it the reader head of VETF (hence termed the CRBD reader, FIG. 32b). The CR is essentially a consensus sequence of 15 A nucleotides, interrupted by a TG dinucleotide^{22,26} (FIG. 32d, FIG. 35a). Arg370 and Gln375 engage in base-specific H-bonding that involves the bases of the TG motif on the non-template strand and the complementary AC dinucleotide on the opposing template strand (FIG. 32c, 32d). By this means, VETF1 anchors the promoter in a defined position relative to the polymerase cleft. The CR displays a high propensity for A nucleotides downstream of the TG motif (FIG. 32d, FIG. 35a). Consistent with this, it was found that only the C5 methyl groups of the corresponding complementary T nucleotides at positions −18 and −17 of the template strand can interact with the reader head by stacking with Tyr376. Promoter binding in the opposite direction would imply an unfavorable contact of Tyr376 with adenine bases (FIG. 32c) and thus a single promoter direction is coerced. By this means, the CRBD-DNA interaction ensures the i) identification of the CR, ii) alignment of the CR relative to the polymerase cleft, and iii) enforcement of transcription directionality. The CRBD is thus is the main control element of the transcription initiation process.

Unusual DNA Binding by the TBP-Like Domain of VETF1

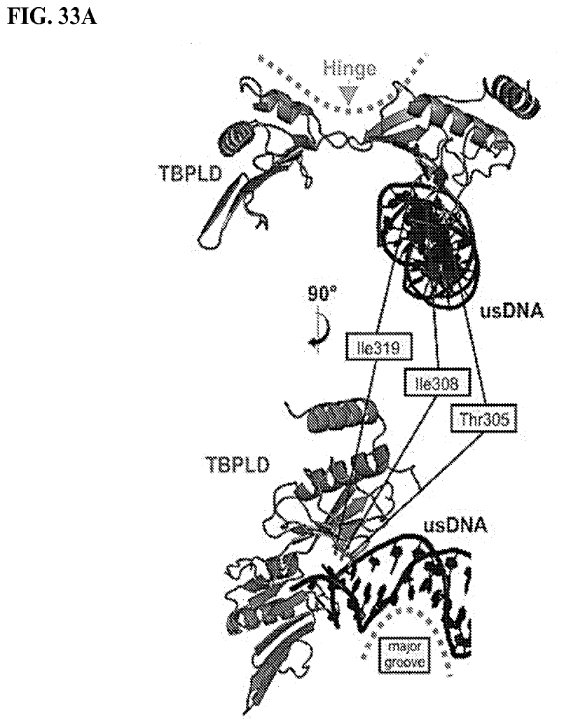

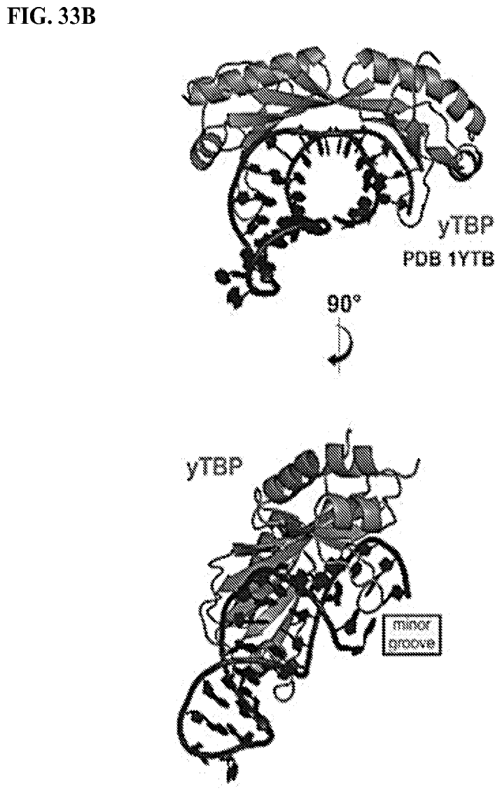

Our structure identified VETF1 as a TBP-like protein (TBPLP). Members of the TBPLD family had previously been identified solely by means of sequence homology. However, VETF1 stands apart from previously known TBPLPs because of its extremely divergent sequence that until now had prevented its classification as such. To compare their structures and binding modes, the VETF1 TBPLD—upstream DNA module (FIG. 33a) was aligned with the yeast TBP-TATA-box crystal structure (FIG. 33b). The TBPLD of VETF1 features the characteristic saddle structure that was previously described for TBP²⁷⁻³⁰, however, the evolutionary conserved symmetry of TBP^{31,32} appears broken. Furthermore, unlike TBP, which contacts TATA box symmetrically, VETF1 binds the promoter asymmetrically and sequence-independently solely through its C-terminal TBP lobe. Most strikingly, the TBPLD inserts into the DNA major groove, contrary to the canonical binding mode of TBP which inserts into the minor groove. In accordance with this observation, the two strictly conserved pairs of DNA-intercalating phenylalanine residues on each lobe of TBP²⁷⁻³⁰ are absent in the TBPLD. Still, the TBPLD induces a pronounced DNA bend via the intercalation of aliphatic, rather than aromatic, sidechains (FIG. 33a). In agreement with the fundamentally different binding mode of the TBPLD, a consensus TATA box is absent from vaccinia early promoters²².

Transition of Complete vRNAP to PIC

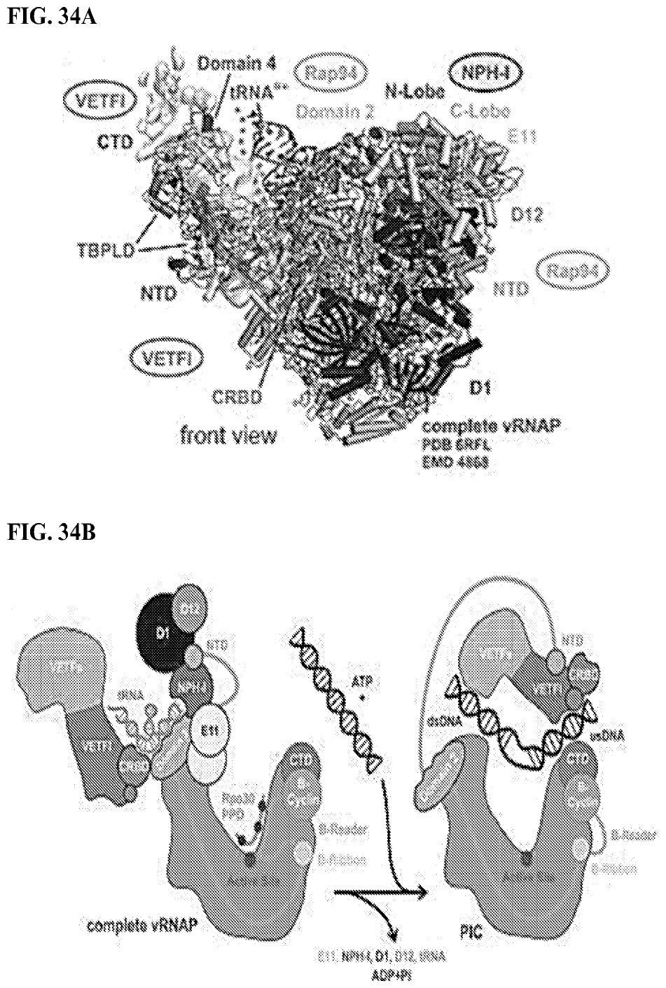

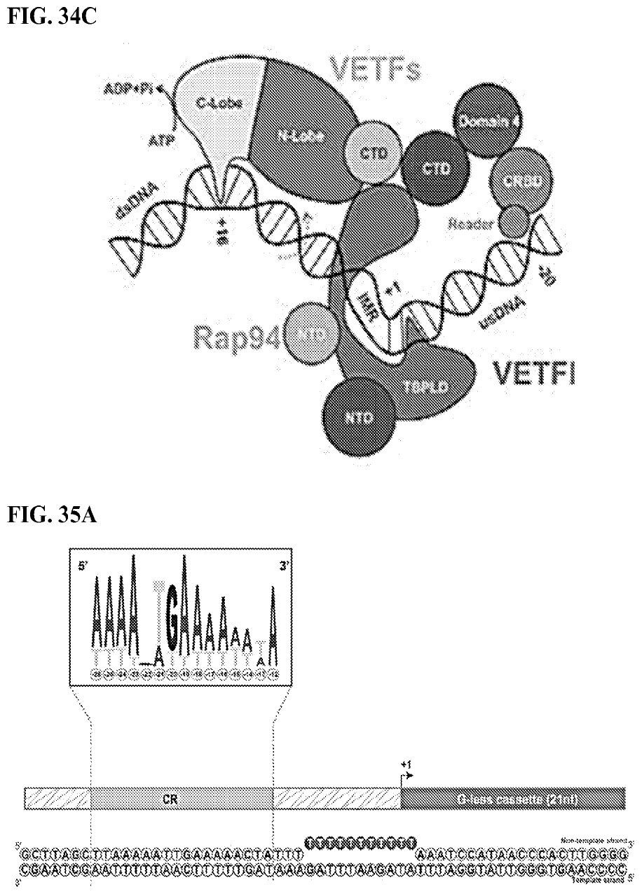

The complete vRNAP is the predominant polymerase complex found in infected cells and necessary and sufficient to carry out the entire early transcription process. It was hypothesized¹⁰ that it is packaged into virions as a pre-assembled unit to promote the restart of transcription in the next infection cycle. To approach the temporal order of events that occur in the transformation of the complete vRNAP to the PIC, both structures were compared. VETF is already present in the complete vRNAP, yet defined density could only be observed for the VETF1 CRBD whereas the remaining parts of VETF were mobile (FIG. 34a). Under the assumption that the adjacent TBPLD is connected flexibly to the CRBD, the diffuse residual density was docked in the vRNAP reconstruction with the VETF1 coordinates extracted from the PIC model, resulting in reasonable overlap. In the resulting structure of the complete vRNAP (FIG. 34a, 34b) VETF1 displays a flexible contact to the tRNA^{Gln}. A comparison with the PIC structure reveals major reconfigurations (FIG. 34b), as all associated factors from the complete vRNAP except for the VETF heterodimer and Rap94 are released. This underlines the importance of complete vRNAP as a viral packaging complex and the high plasticity of vaccinia transcriptional complexes.

### DISCUSSION

Our structure of the vaccinia PIC in the initially melted state provided insight into the unique mode of poxvirus transcription initiation. The CRBD of VETF1 is the decisive element for the sequence-specific recognition of early promoters. Of note, the CRBD constitutes a thus far unknown DNA-binding fold, which is stabilized by three disulfide bridges. Cystine formation in the CRBD may be introduced by vaccinia-encoded enzymes³³ rather than host factors, which localize in the endoplasmic reticulum. The TBPLD of VETF1, located adjacent to the CRBD introduces a sharp DNA bend, likely to be the nucleation site for melting of the IMR. TBPLDs had been bioinformatically predicted in a large number of proteins but their structure and mode of DNA binding remains elusive. Unexpectedly, the TBPLD of VETF1 displays an asymmetric rather than symmetric binding mode as shown for TBP in the context of Pol II transcription. Asymmetric binding to DNA has also been postulated to occur in the context of Pol I and Pol III PICs and may be also a feature of other TBPLDs^{31,34,35}.

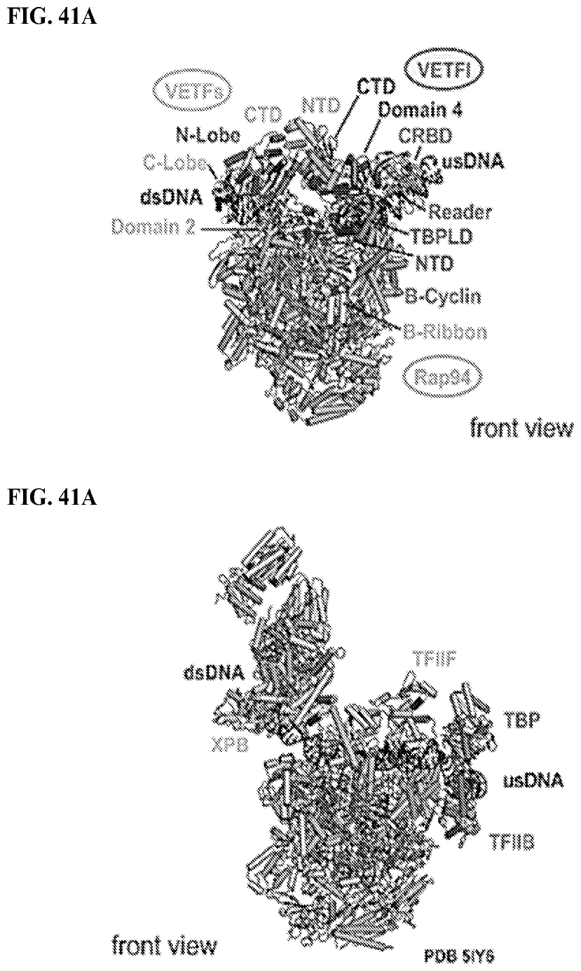

A structure-based comparison to eukaryotic transcription systems pins down obvious differences in the bound transcription factors whereas similar positioning of the bound promoter relative to the core polymerases is observed in all PICs. Likewise, the positions of the B-homology region of Rap94 in the vaccinia PIC and the corresponding domain of TFIIB in the Pol II PIC^{36,37} overlap (FIG. 41). However, whereas TFIIB directly contacts the promoter, the B-homology region in Rap94 does not bind DNA (FIGS. 31a, 31b).

Some features of in the distal section of the DNA path also appear to be conserved and a common principle might be the binding of a helicase transcription factor to the downstream promoter. It appears plausible that the helicase domains of VETFs and of the TFIIH subunit XPB (FIG. 41) are functional counterparts³⁸. However, in contrast to a recent study describing a Pol II PIC intermediate immediately prior to the initially melted state³⁹, underwinding of the DNA duplex in the Vaccinia PIC was not observed. This could be explained by the simple fact that the melted IMR has absorbed a presumable previous negative twist during the melting process.

On the promoter upstream side, it was noted that an architectural relationship of the VETF1 promoter complex and the positioning of the Rap94 CTD with the TBP/TFIIF module on the DNA in the Pol-II PIC. This notion is corroborated by the fact that despite their fundamentally different binding modes both, TBP and the VETF1 TBPLD, induce a strong bend of the DNA. Thus, although the architecture of the vaccinia PIC differs fundamentally from its nuclear counterparts (FIG. 41) with respect to the involved transcription factors, basic architectural features are conserved.

Based on the data reported here and prior findings³⁹ for the Pol II system, a mechanism was proposed for melting of the vaccinia early promoter (FIG. 34c): (i) The CRBD of VETF1 binds the promoter at the CR, thereby enforcing directionality. (ii) VETFs pulls the DNA in an ATP-dependent reaction towards the vRNAP clamp and lobe, analogous to the XPB helicase in the Pol II system. (iii) The promoter DNA becomes underwound and bent by 80° towards the C-lobe of VETFs, exposing bases for an interaction with the latter. (iv) The tip of the C-terminal lobe of the VETF1 TBPLD intercalates upstream of the IMR, inducing a second sharp bend in the promoter. (v) This bend triggers the initial melting event around the transcription start site, and the IMR absorbs the negative twist of the adjacent DNA segments. Thus, these results and those of the accompanying Examples describing structures of vaccinia initially transcribing complexes provide a comprehensive picture of vaccinia transcription initiation.

Methods

vRNAP Purification from Recombinant Vaccinia Virus GLV-1h439

The generation of GLV-1h439 has been described previously¹⁰. For vRNAP purification, Hela S3 cells were cultured in Dulbecco's modified Eagle Medium (DMEM), containing 10% fetal bovine serum at 37° C. in a present of 5% CO₂. Cells were grown up to 80-90% of confluency and then infected with purified GLV-1h439 with a multiplicity of infection (MOI) of 1.2. After 24 h, the infected cells were pelleted and resuspended in lysis buffer (50 mM HEPES, pH 7.5, 150 mM NaCl, 1.5 mM MgCl₂, 0.5% [v/v] NP-40, 1 mM DTT, and complete EDTA-free protease inhibitor cocktail (Sigma-Aldrich). The soluble supernatant of the cellular extract was incubated for 3h at 4° C. with anti-FLAG Agarose beads (Sigma Aldrich). Beads were washed four times with buffer containing 50 mM HEPES, pH 7.5, 150 mM NaCl, 1.5 mM MgCl₂, 0.1% [v/v] NP-40, 1 mM DTT, equilibrated with elution buffer (50 mM HEPES, pH 7.5, 150 mM NaCl, 1.5 mM MgCl₂ and 1 mM DTT) and eluted with a 200 μg/ml solution of 3×FLAG Peptide (Sigma-Aldrich). The eluate was analyzed by SDS-PAGE and the protein components were identified by mass spectrometry (see also FIG. 35b). Approx. 50 μg of purified vRNAP was obtained from one 15 cm petri-dish of Hela S3 cells infected with the virus.

Reconstitution of Promoter Bound vRNAP Complexes

A synthetic double stranded DNA oligonucleotide scaffold mimicking the vaccinia virus early promoter region was generated by annealing of two partially complementary DNA oligonucleotides (see FIG. 35a). Annealing was performed in buffer containing 100 mM NaCl, 20 mM HEPES, pH 7.5, and 3 mM MgCl₂ by heating the mixture to 95° C. for 5 min followed by slowly cooling down to room temperature. The resulting double stranded DNA oligo was precipitated by isopropanol and the dry pellet was resuspended in 1× resuspension buffer (10 mM Tris-HCl, pH 8.0, 1 mM EDTA).

For reconstitution of promoter-bound vRNAP complexes approx. 1 pmol of [³²P]-labeled DNA promoter-scaffold was incubated for 30 min at 30° C. with the indicating amount of vRNAP in the presence of 1 mM of the indicated NTPs (FIG. 35). Reconstitutions were analyzed by native gel electrophoresis (4% acrylamide and 0.13% bis-acrylamide, 25 mM Tris-HCl pH 7.4, 25 mM Boric acid and 0.5 mM EDTA) at 4° C. For large-scale reconstitution of promoter/vRNAP complexes, purified vRNAP was concentrated in a Viva-spin (Sartorius). A total of 400 μg of vRNAP was incubated with a 60 fold-molar excess of the DNA scaffold in reconstitution buffer (50 mM NaCl, 10 mM Tris-HCl, pH 7.5, 5 mM MgCl₂ and 1 mM DTT) in the presence of ATP and UTP (1 mM each) for 30 min at 30° C. The mixture was separated by 10%-30% sucrose gradient centrifugation (16h, 35.000 rpm, Beckman 60Ti rotor, 4° C.). Gradient fractions were collected manually and analyzed by SDS-PAGE followed by Silver staining and ethidium-bromide staining to visualize the proteins and the DNA scaffold, respectively. The indicated fractions (FIG. 35) were used for cryo-EM analysis after buffer exchange with modified reconstitution buffer (100 mM NaCl, 10 mM Tris-HCl, pH 7.5, 5 mM MgCl₂ and 1 mM DTT) in a Vivaspin concentrator (Sartorious; 10 MW cut-off).

Transcription Assay

A plasmid containing an early vaccinia virus promoter fused with a G-less cassette (termed psB24) was used. For vRNAP catalyzed transcription assays, 400 ng of SmaI-linearized pSB24 template was incubated with 100 μg vRNAP in buffer containing 40 mM Tris-HCl, pH 7.9, 1 mM DTT, 2 mM spermidine, 6 mM MgCl₂, 1 mM ATP, CTP and GTP, 0.1 mM UTP and 20 μCi [³²P]-UTP, and 80 μM S-adenosyl-methionine. The transcription mixture was incubated at 30° C. for the indicated time points. Radio-labelled RNA transcripts were tryzol-extracted, precipitated by isopropanol and analyzed by denaturing 5% urea polyacrylamide gel electrophoresis. Transcripts were subsequent visualized by autoradiography.

Cryo-EM and Model Building

Following sucrose gradient purification, the indicated fractions (see FIG. 35) was diluted 1:50 with a buffer containing 10 mM Tris-HCl, pH 7.5, 100 mM NaCl, 5 mM MgCl₂ and 1 mM DTT, and centrifuged in a Vivaspin concentrator to remove the sucrose. For cryo-EM analysis the sample was centrifuged for 40 min at 10,000 rpm. For cryo-EM data collection, R 1.2/1.3 holey carbon grids (Quantifoil) were glow-discharged for 90 s (Plasma Cleaner model PDC-002. Harrick Plasma Ithaca, N.Y./USA) at medium power, and 3.5 μl of C2 sample was applied inside a Vitrobot Mark IV (FEI) at 4° C. and 100% relative humidity. Grids were blotted for 3 s and with blot force 5 and plunged into liquid ethane. Cryo-EM datasets comprising 10816 (dataset 1), 9878 (dataset 2), and 3640 (dataset 3) micrographs, respectively, were collected from three different grids with a Thermo Fisher Titan Krios G3 and a Falcon III camera (Thermo-Fischer). Data was acquired with EPU at 300 keV and a primary magnification of 75,000 (calibrated pixel size 1.0635 Å) in movie-mode with 47 fractions per movie and counting of the electron signal. The total exposure was 77.5 e/Å² for 75 sec, with 2 exposures per hole.

Dose-weighted, motion-corrected sums of the micrograph movies were calculated with Motioncorr2 (Zheng et al., 2017). The contrast-transfer function of each micrograph was fitted with Relion 3.1. An initial set of 25,000 particles was picked with the Gaussian picker and subjected to three rounds of 2D-Classification in Relion (Zivanov et al., 2018) to clean up the dataset. Eight reasonable class averages were selected as templates for subsequent automated particle picking within Relion and a total of 300,000 particles were picked using the Relion autopicker. After a second round of 2D classification, 3D classification was performed using the vRNAP core structure as template. Particles belonging to the PIC were selected and 2D classes for autopicking were calculated. The resulting three particle stacks, one for each dataset, were cleaned up individually by four rounds of 2D classification each, and contained 1,064,795 (dataset 1), 1,205,746 (dataset 2), and 323,776 (dataset 3) good particles. Each particle stack was then subjected to 3D classification and particles that fell in the defined PIC class were selected. The PIC particle stacks of the three datasets were then united into a single stack, and CTF refinement, followed by a consensus 3D refinement, was performed. This united particle stack was then subjected to a focused 3D classification with a mask that selected for VETF and DNA. Two of the resulting three classes yielded high-resolution reconstructions of VETF and DNA in minimally divergent conformations (FIG. 35A). The particles from the two good classes were then forwarded to a Multibody (MB) refinement in Relion, either pooled or separately. The MB refinement was performed with two bodies, representing VETF and DNA and core vRNAP. It was noted that minor variations of the mask pairs resulted in the improvement of particular regions of the reconstruction. The MB refinement was therefore repeated with 11 more mask pairs and combined the resulting maps with Phenix.combine_focused_maps to create a single, optimal map for refinement.

To build the PIC model, the vRNAP core excluding the Rpo30 phospho-peptide domain (PPD) was extracted from the complete vRNAP structure (PDB 6RFL) and docked into the cryo-EM density map. Within the residual density, the path of the DNA was identified and manually docked with section-wise stretches of ideal B-DNA. VETF was then traced de novo in COOT 0.9. To this end, the SNF2 helicase core of VETFs was located and built first, followed by well-defined regions VETF1. The resulting partial model was initially refined with Phenix.real_space_refine and forwarded to Phenix.combine_focused_maps to create a stitched, optimal map. The VETF model was then completed manually and the full polypeptide chains of both, VETFs and VETF1, could be modelled. Finally, residual density was identified as the relocated Rap94 NTD, and the DNA sequence was assigned. The resulting model was manually optimized with the real-space-refinement routine of COOT 0.9 and subjected again to refinement with Phenix.real_space_refine including ADP refinement steps. During refinement, secondary structure and mild Ramachandran restraints were imposed. After four further cycles of manual inspection and automated refinement, the refinement converged, and a model with excellent stereochemistry and good correlation with the cryo-EM map was obtained (Table 2).

TABLE 2
Cryo-EM data collection, single-particle reconstruction
and model refinement statistics.

| | PIC |
|---|---|
| Data collection | |
| Voltage (kV) | 300 |
| Total electron dose (e⁻/Å²) | 77.5 |
| Number of fractions | 47 |
| Exposure time (s) | 75 |
| Number of movies per hole | 2 |
| Number of movies in dataset* | 10,816/9,878/3,640 |
| Particles (automatically selected)* | 6,732,253/6,385,645/1,888,776 |
| Particles (in final reconstruction) | 181,788 |
| Pixel size (Å) | 1.0635 |
| Defocus range (μm) | −1.0 to −2.2 |
| Detector | Falcon III camera, |
| | counting mode |
| Reconstruction (Relion)^{#} | |
| Accuracy of rotations (°) | 0.53 (0.52) |
| Accuracy of translations (pixels) | 0.32 (0.35) |
| Resolution (Å) | 2.64 (3.15) |
| Map sharpening B-factor (Å²) | −60 (−60) |
| Model composition | |
| Non-hydrogen atoms | 45175 |
| Protein residues | 5348 |
| Nucleic acid residues | 84 |
| Ions | Zn:4, Mg:1 |
| Refinement (Phenix.real_space_refine) | |
| Map CC (around atoms) | 0.79 |
| RMS deviations | |
| Bond lengths (Å) | 0.006 |
| Bond angles (°) | 0.83 |
| Validation | |
| All-atom clashscore | 2.81 |
| Rotamer outliers (%) | 0.41 |
| C-beta deviations | 0 |
| Ramachandran plot (%) | |
| Outliers | 0.0 |
| Allowed | 2.8 |
| Favored | 97.2 |

*Values for dataset 1/dataset 2/dataset 3
^{#}Values for consensus refinement. Values in parentheses for two-body multibody refinement, body 2 (VETF + DNA).

### REFERENCES FOR EXAMPLE 4

1. Shchelkunov, S. N. An increasing danger of zoonotic orthopoxvirus infections. PLoS Pathog 9, e1003756 (2013).
2. Lewis-Jones, S. Zoonotic poxvirus infections in humans. Curr Opin Infect Dis 17, 81-9 (2004).
3. Grant, R., Nguyen, L. L. & Breban, R. Modelling human-to-human transmission of monkeypox. Bull World Health Organ 98, 638-640 (2020).
4. Rohrmann, G., Yuen, L. & Moss, B. Transcription of vaccinia virus early genes by enzymes isolated from vaccinia virions terminates downstream of a regulatory sequence. Cell 46, 1029-35 (1986).
5. Broyles, S. S. & Moss, B. Homology between RNA polymerases of poxviruses, prokaryotes, and eukaryotes: nucleotide sequence and transcriptional analysis of vaccinia virus genes encoding 147-kDa and 22-kDa subunits. Proc Natl Acad Sci USA 83, 3141-5 (1986).
6. Ensinger, M. J., Martin, S. A., Paoletti, E. & Moss, B. Modification of the 5′-terminus of mRNA by soluble guanylyl and methyl transferases from vaccinia virus. Proc Natl Acad Sci USA 72, 2525-9 (1975).
7. Broyles, S. S., Yuen, L., Shuman, S. & Moss, B. Purification of a factor required for transcription of vaccinia virus early genes. J Biol Chem 263, 10754-60 (1988).
8. Yuen, L., Davison, A. J. & Moss, B. Early promoter-binding factor from vaccinia virions. Proc Natl Acad Sci USA 84, 6069-73 (1987).
9. Hillen, H. S. et al. Structural Basis of Poxvirus Transcription: Transcribing and Capping Vaccinia Complexes. Cell 179, 1525-1536 e12 (2019).
10. Grimm, C. et al. Structural Basis of Poxvirus Transcription: Vaccinia RNA Polymerase Complexes. Cell 179, 1537-1550 e19 (2019).
11. Gershon, P. D. & Moss, B. Early transcription factor subunits are encoded by vaccinia virus late genes. Proc Natl Acad Sci USA 87, 4401-5 (1990).
12. Niles, E. G., Lee-Chen, G. J., Shuman, S., Moss, B. & Broyles, S. S. Vaccinia virus gene D12L encodes the small subunit of the viral mRNA capping enzyme. Virology 172, 513-22 (1989).
13. Shuman, S., Broyles, S. S. & Moss, B. Purification and characterization of a transcription termination factor from vaccinia virions. J Biol Chem 262, 12372-80 (1987).
14. Broyles, S. S. & Moss, B. Sedimentation of an RNA polymerase complex from vaccinia virus that specifically initiates and terminates transcription. Mol Cell Biol 7, 7-14 (1987).
15. Liu, X., Bushnell, D. A., Wang, D., Calero, G. & Kornberg, R. D. Structure of an RNA polymerase II-TFIIB complex and the transcription initiation mechanism. Science 327, 206-9 (2010).
16. Ahn, B. Y., Gershon, P. D. & Moss, B. RNA polymerase-associated protein Rap94 confers promoter specificity for initiating transcription of vaccinia virus early stage genes. J Biol Chem 269, 7552-7 (1994).
17. Ahn, B. Y. & Moss, B. RNA polymerase-associated transcription specificity factor encoded by vaccinia virus. Proc Natl Acad Sci USA 89, 3536-40 (1992).
18. Broyles, S. S. & Fesler, B. S. Vaccinia virus gene encoding a component of the viral early transcription factor. J Virol 64, 1523-9 (1990).
19. Broyles, S. S. & Moss, B. DNA-dependent ATPase activity associated with vaccinia virus early transcription factor. J Biol Chem 263, 10761-5 (1988).
20. Shuman, S. & Hurwitz, J. Mechanism of mRNA capping by vaccinia virus guanylyltransferase: characterization of an enzyme—guanylate intermediate. Proc Natl Acad Sci USA 78, 187-91 (1981).
21. Paoletti, E. & Moss, B. Two nucleic acid-dependent nucleoside triphosphate phosphohydrolases from vaccinia virus. Nucleotide substrate and polynucleotide cofactor specificities. J Biol Chem 249, 3281-6 (1974).
22. Davison, A. J. & Moss, B. Structure of vaccinia virus early promoters. J Mol Biol 210, 749-69 (1989).
23. Cassetti, M. A. & Moss, B. Interaction of the 82-kDa subunit of the vaccinia virus early transcription factor heterodimer with the promoter core sequence directs downstream DNA binding of the 70-kDa subunit. Proc Natl Acad Sci USA 93, 7540-5 (1996).
24. Baldick, C. J., Jr., Cassetti, M. C., Harris, N. & Moss, B. Ordered assembly of a functional preinitiation transcription complex, containing vaccinia virus early transcription factor and RNA polymerase, on an immobilized template. J Virol 68, 6052-6 (1994).
25. Kitano, K., Kim, S. Y. & Hakoshima, T. Structural basis for DNA strand separation by the unconventional winged-helix domain of RecQ helicase WRN. Structure 18, 177-87 (2010).
26. Yang, Z., Bruno, D. P., Martens, C. A., Porcella, S. F. & Moss, B. Genome-wide analysis of the 5′ and 3′ ends of vaccinia virus early mRNAs delineates regulatory sequences of annotated and anomalous transcripts. J Virol 85, 5897-909 (2011).
27. Nikolov, D. B. & Burley, S. K. 2.1 A resolution refined structure of a TATA box-binding protein (TBP). Nat Struct Biol 1, 621-37 (1994).
28. Kim, J. L. & Burley, S. K. 1.9 A resolution refined structure of TBP recognizing the minor groove of TATAAAAG. Nat Struct Biol 1, 638-53 (1994).
29. Kim, Y., Geiger, J. H., Hahn, S. & Sigler, P. B. Crystal structure of a yeast TBP/TATA-box complex. Nature 365, 512-20 (1993).
30. Kim, J. L., Nikolov, D. B. & Burley, S. K. Co-crystal structure of TBP recognizing the minor groove of a TATA element. Nature 365, 520-7 (1993).
31. Blombach, F. & Grohmann, D. Same but different: The evolution of TBP in archaea and their eukaryotic offspring. Transcription 8, 162-168 (2017).
32. Hobbs, N. K., Bondareva, A. A., Barnett, S., Capecchi, M. R. & Schmidt, E. E. Removing the vertebrate-specific TBP N terminus disrupts placental beta2m-dependent interactions with the maternal immune system. Cell 110, 43-54 (2002).
33. Senkevich, T. G., White, C. L., Koonin, E. V. & Moss, B. Complete pathway for protein disulfide bond formation encoded by poxviruses. Proc Natl Acad Sci USA 99, 6667-72 (2002).
34. Ravarani, C. N. J. et al. Molecular determinants underlying functional innovations of TBP and their impact on transcription initiation. Nat Commun 11, 2384 (2020).
35. Kramm, K., Engel, C. & Grohmann, D. Transcription initiation factor TBP: old friend new questions. Biochem Soc Trans 47, 411-423 (2019).
36. Schilbach, S. et al. Structures of transcription pre-initiation complex with TFIIH and Mediator. Nature 551, 204-209 (2017).
37. Sainsbury, S., Bernecky, C. & Cramer, P. Structural basis of transcription initiation by RNA polymerase II. Nat Rev Mot Cell Biol 16, 129-43 (2015).
38. Kolesnikova, O., Radu, L. & Poterszman, A. TFIIH: A multi-subunit complex at the cross-roads of transcription and DNA repair. Adv Protein Chem Struct Biol 115, 21-67 (2019).
39. Dienemann, C., Schwalb, B., Schilbach, S. & Cramer, P. Promoter Distortion and Opening in the RNA Polymerase II Cleft. Mol Cell 73, 97-106 e4 (2019).
40. Murakami, K. et al. Uncoupling Promoter Opening from Start-Site Scanning. Mol Cell 59, 133-8 (2015).
41. Tan, S., Hunziker, Y., Sargent, D. F. & Richmond, T. J. Crystal structure of a yeast TFIIA/TBP/DNA complex. Nature 381, 127-51 (1996).
42. Nogales, E., Louder, R. K. & He, Y. Structural Insights into the Eukaryotic Transcription Initiation Machinery. Annu Rev Biophys 46, 59-83 (2017).

### Example 5. Structural Basis of Poxvirus Transcription Initiation and Promoter Escape

A virus-encoded DNA-dependent RNA polymerase (vRNAP) facilitates gene expression of poxviruses in the cytoplasm of infected cells. In the accompanying example are described structures unrevealing how vRNAP of the poxvirus vaccinia recognizes an early viral promotor and forms the pre-initiation complex (PIC). Here cryo-EM was used to investigate the structural basis of PIC conversion into the transcription initiation mode. These structures uncover the mechanism of promotor hand-over from the viral early transcription factor (VETF) to vRNAP, capture of the template strand, promoter scrunching, and promoter escape. In the transition of pre-initiation into the initiation mode, the phospho-peptide domain (PPD) of the vRNAP subunit Rpo30 mimics the promoter template strand and pairs with the B-reader domain of Rap94 in the active cleft. During single-strand capture, the PPD is replaced by the template strand and the B homology domain becomes mobile. In the late initially transcribing phase, the viral helicase NPH-I binds to the upstream promoter and Rap94 undergoes major rearrangements. The resulting structure resembles the Pol II transcription-coupled repair (TCR) initiation complex and suggests an ATP-dependent mechanism of upstream promoter scrunching by NPH-I. By this mechanism, an energy-loaded intermediate is produced, that transitions into the productive elongation complex. Together with the accompanying Example describing the PIC formation, a complete picture of poxvirus transcription initiation emerges.

Protein-coding genes are transcribed in the nucleus of eukaryotes by the multi-subunit DNA-dependent RNA polymerase Pol II. The structural basis of Pol II transcription is well studied. Initiation occurs through the ordered interplay of transcription factors with promotor sequences that allows Pol II recruitment and formation of a pre-initiation complex (PIC). The PIC melts the promoter, enforces exact positioning of the transcription machinery, and defines the template-strand. The latter two processes are dependent on TFIIB, which screens for the transcription start site by accessing the template strand tunnel [Liu et al.]. The transcript length of 7 nucleotides marks a decision point, beyond which Pol II transitions into processive RNA synthesis. The passing of the decision point is referred to as promoter escape and facilitated by an energy-loaded transition state in which melted downstream DNA is ‘scrunched’ into the polymerase.

Many DNA viruses make use of the Pol II transcription machinery and thus enter the nuclear compartment during infection. A remarkable exception is the family of poxviruses, whose members can be highly pathogenic but also serve as vaccine and agents in cancer therapies. Their entire life cycle is confined to the cytoplasm of infected cells and thus depends on virus-encoded factors. Studies with the poxvirus vaccinia led to the identification of a multi-subunit DNA-dependent RNA polymerase (vRNAP) consisting of eight core subunits (Rpo)¹⁻¹⁰. To enable viral gene expression, the core vRNAP, which shares structural similarities to Pol II cooperates with unique virus-encoded transcription factors. Early transcription, which accounts for the expression most viral genes critically depends on the multidomain factor Rap94¹¹. It connects the core vRNAP with the helicase NPH-I^{12,13}, the early transcription factor VETF^{14,15} and the capping enzyme¹⁶⁻¹⁸. One domain of this factor is partially homologous to TFIIB, suggesting a role in transcription start site recognition. Recently the structure of the vaccinia complete vRNAP¹⁹ that unites all early initiation factors and is necessary and sufficient for early transcription was described. In the accompanying example is described the structure of the pre-initiation complex (PIC) on an early viral promotor and identify VETF as the key factor for promotor recognition. Using cryo-EM the structures of different stages of the vaccinia early transcription apparatus are now reported on which illuminate how the PIC is converted to the initiation complex and escapes the promoter^{20,21}.

Cryo-EM of Vaccinia Transcription Initiation Complexes

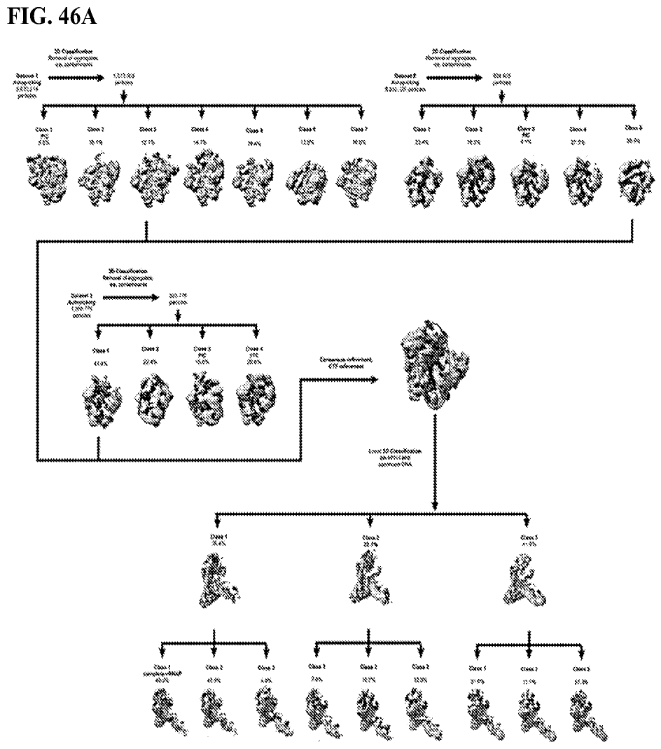

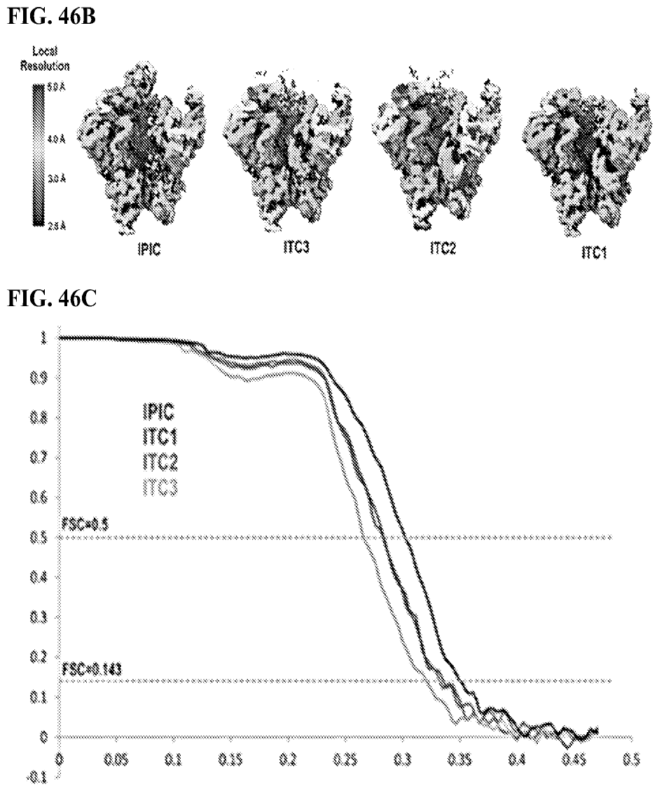

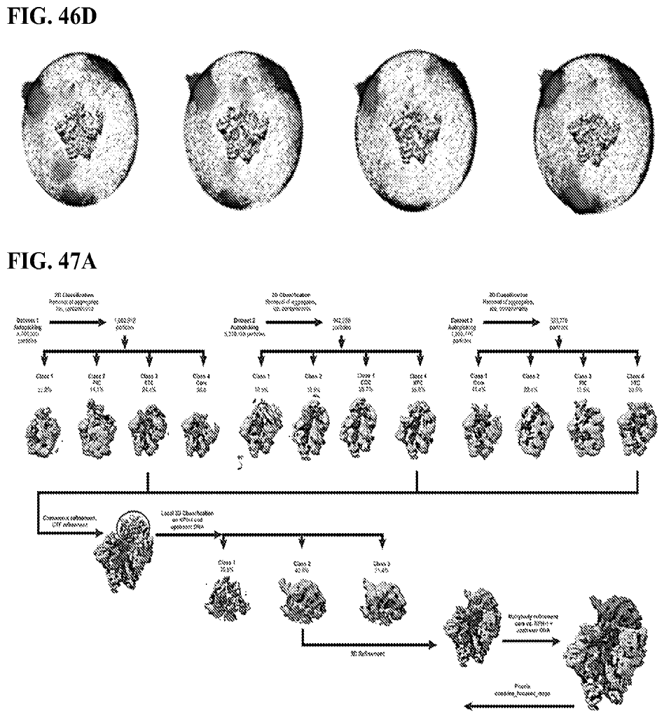

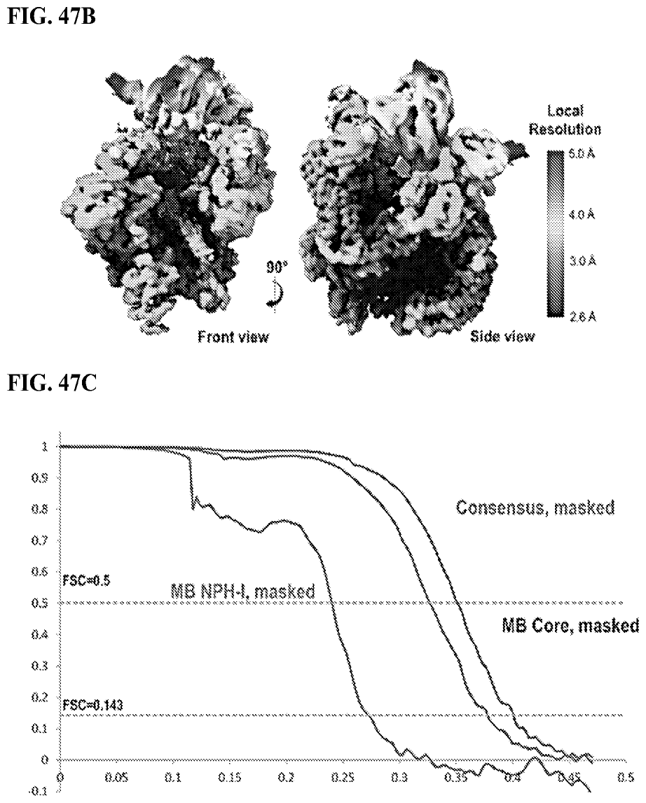

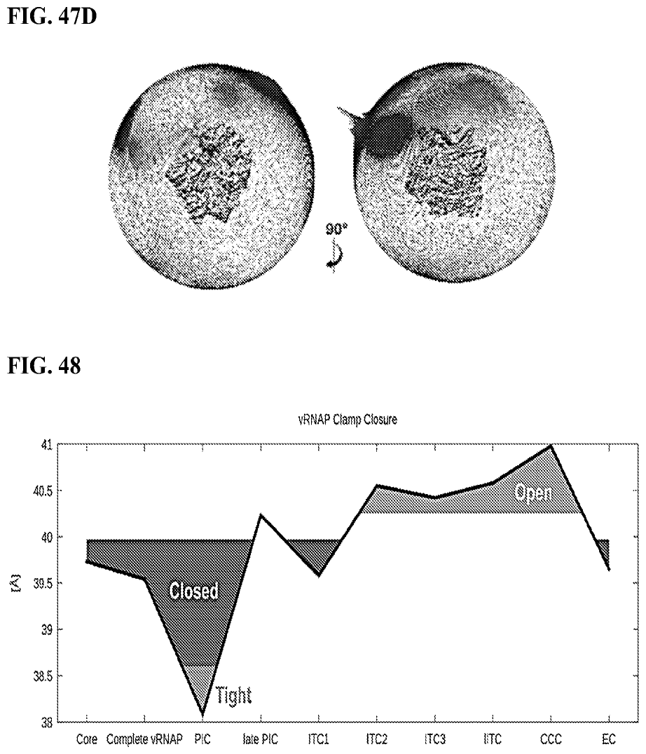

Transcription-active complete vRNAP was isolated from HeLa cells infected with an engineered vaccinia strain expressing the FLAG-tagged vRNAP subunit Rpo132¹⁹. Upon incubation with a synthetic early promotor scaffold complexes were formed in an ATP/UTP dependent manner (FIG. 46). DNA-bound vRNAP complexes were isolated by sucrose gradient centrifugation (FIG. 46) and analyzed by cryo-EM. After extensive 3D classification, distinct promotor-containing vRNAP particle classes could be separated. Based on their composition and the state of the bound template and transcription products, two classes in each dataset were identified as initial transcribing complexes (ITC) (FIG. 46a). Further focused two-fold subclassification of the downstream DNA channel region resolved different ITC-like subclasses that represent the late pre-initiation complex (IPIC) and 3 different initially transcribing complexes (ITC1-3) (FIG. 46a, rows 3 and 4). In addition, a particle class with the helicase NPH-1 bound to the upstream region next to the core vRNAP cleft was identified as a late initially transcribing complex (° ITC, FIG. 47). These identified particle classes thus represent different transcription stages from pre-initiation (see also accompanying Example) to elongation.

Structure of the Late Pre-Initiation Complex

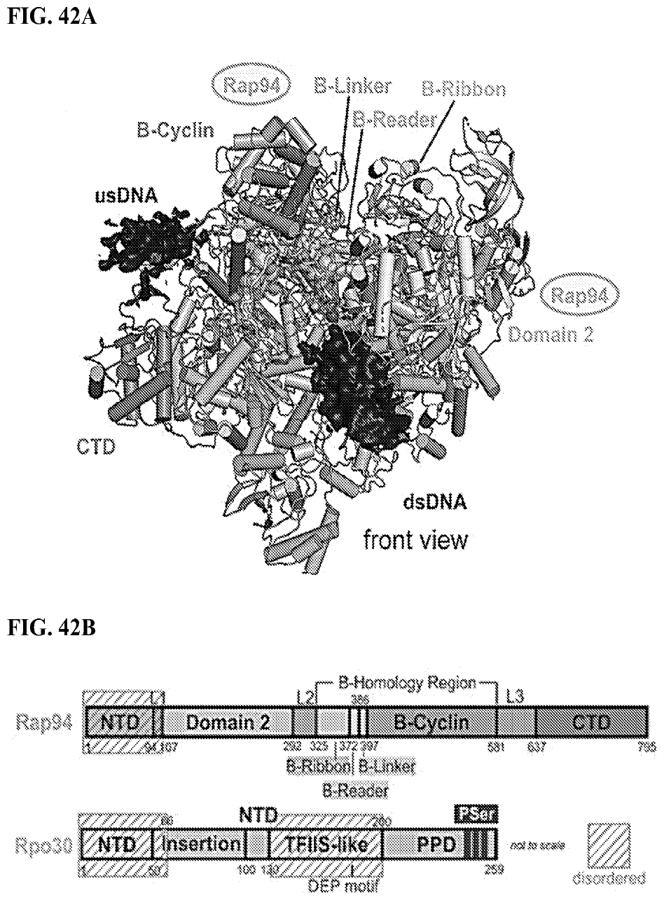

Particles from class 1 subclass 2 yielded a reconstruction at 3.0 Åresolution (FIG. 46a and Table 3). The density could be docked with the complete vRNAP model but except for Rap94 no early transcription factors were present¹⁹. Disordered density corresponding to DNA is visible upstream next to the Rap94 CTD and within the downstream DNA channel. These sites roughly coincide with the DNA anchor points on the core cRNAP observed in the PIC (see accompanying Example). However, no density for the DNA transcription bubble or nascent RNA was detected at this stage in the active cleft (FIG. 42A). Instead, awell-defined density was found for the phospho-peptide domain (PPD) of Rpo30 in the active site cleft in a similar conformation as in the complete vRNAP¹⁹. It follows the path of the template and non-template strand in the elongation complex (EC), allows pairing with the B-reader of Rap94¹⁹ (FIG. 42B) and enables single strand capture at later stages (see below). Based on these observations it was concluded that this particle is a late state of the PIC (1PIC) in which VETF has been expelled, the melted promoter has been handed over to the core vRNAP but transcription has not yet been initiated.

TABLE 3
Cryo-EM data collection, single-particle reconstruction and model refinement statistics

| | lPIC | ITC1 | ITC2 | ITC3 | lITC |
|---|---|---|---|---|---|
| Data collection | | | | | |
| Voltage (kV) | 300 | | | | |
| Total electron dose | 77.5 | | | | |
| (e⁻/Å²) | 47 | | | | |
| Number of fractions | 75 | | | | |
| Exposure time (s) | 2 | | | | |
| Number of movies per | 10,816/9,878/3,640 | | | | |
| hole | | | | | |
| Number of movies in | | | | | |
| dataset* | | | | | |
| Particles (automatically | 1,513,003/ | 1,513,003/ | 1,513,003/ | 1,513,003/ | 4,766,02/ |
| selected)* | 5,310,128/0 | 5,310,128/0 | 5,310,128/0 | 5,310,128/0 | 5,310,128/ |
| | | | | | 942,258 |
| Particles (in final | 88828 | 133467 | 73035 | 96165 | 278260 |
| reconstruction) | | | | | |
| Pixel size (Å) | 1.0635 | 1.0635 | 1.0635 | 1.0635 | 1.0635 |
| Defocus range (μm) | −1.0 to −2.2 | −1.0 to −2.2 | −1.0 to −2.2 | −1.0 to −2.2 | −1.0 to −2.2 |
| Detector | Falcon III camera, counting mode | | | | |
| Reconstruction (Relion)^{#} | | | | | |
| Accuracy of rotations (°) | 0.50 | 0.55 | 0.56 | | 0.58 (1.20) |
| Accuracy of translations | 0.30 | 0.34 | 0.35 | | 0.39 |
| (pixels) | | | | | |
| Resolution (Å) | 3.0 | 2.9 | 3.2 | 3.0 | 2.5 (3.7) |
| Map sharpening B-factor | −40 (−40) | −40 (−40) | −40 (−40) | −40 (−40) | −40 (−40) |
| (Å²) | | | | | |
| Model composition | | | | | |
| Non-hydrogen atoms | 32727 | 31512 | 31399 | 30881 | 39139 |
| Protein residues | 4034 | 3782 | 3782 | 3782 | 4675 |
| Nucleic acid residues | 0 | 42 | 77 | 11 | 77 |
| Ions | 1 Mg, 4 Zn | 1 Mg, 4 Zn | 1 Mg, 4 Zn | 1 Mg, 4 Zn | 1 Mg, 4 Zn |
| Refinement | | | | | |
| (Phenix.real_space_refine) | | | | | |
| Map CC (around atoms) | 0.87 | 0.87 | 0.82 | 0.88 | 0.84 |
| RMS deviations | | | | | |
| Bond lengths (Å) | 0.004 | 0.005 | 0.003 | 0.009 | 0.005 |
| Bond angles (°) | 0.9 | 1.07 | 0.56 | 1.08 | 0.67 |
| Validation | | | | | |
| All-atom clashscore | 4.3 | 3.0 | 3.4 | 4.3 | 3.5 |
| Rotamer outliers (%) | 1.8 | 2.4 | 1.8 | 2.9 | 1.6 |
| C-beta outliers (%) | | 0.0 | 0.0 | 0.0 | 0.0 |
| Ramachandran plot (%) | | | | | |
| Outliers | 0.0 | 0.0 | 0.0 | 0.0 | 0.0 |
| Allowed | 5.0 | 5.8 | 5.4 | 6.9 | 3.1 |
| Favored | 95.0 | 94.2 | 94.6 | 93.1 | 96.9 |

*Values for dataset 1/dataset 2/dataset 3
^{#}Values for consensus refinement. Values in parentheses for two-body multibody refinement, body 2 (NPH-I + DNA).

Three Structures of the Initially Transcribing Complex

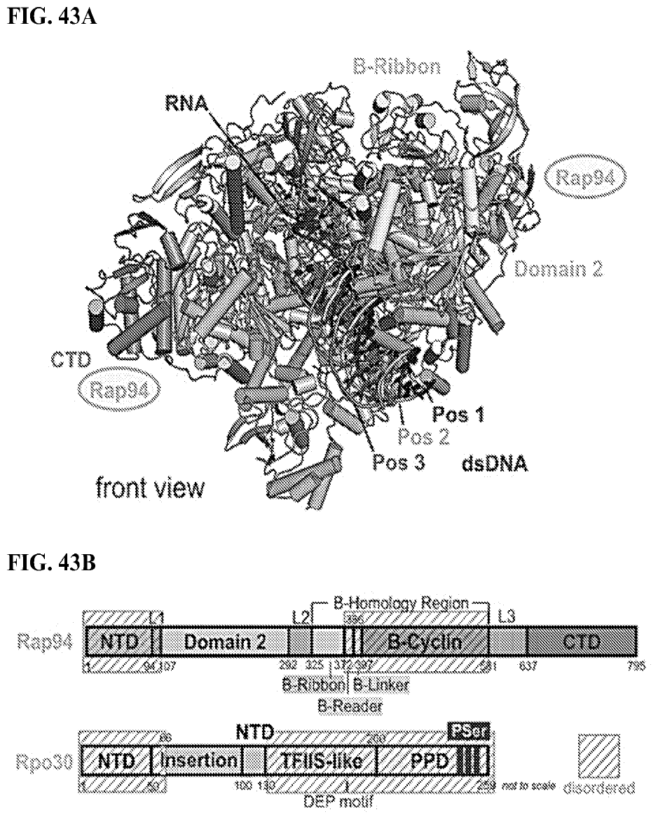

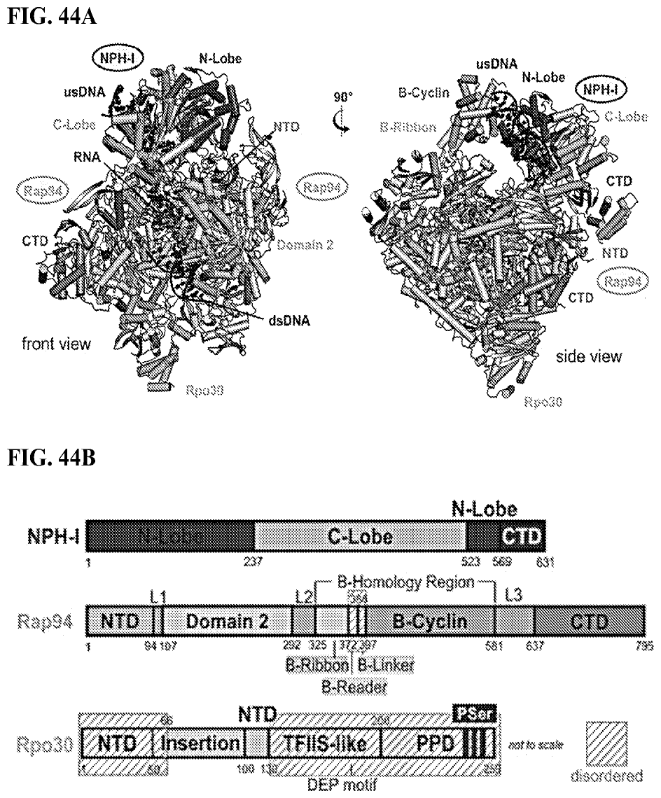

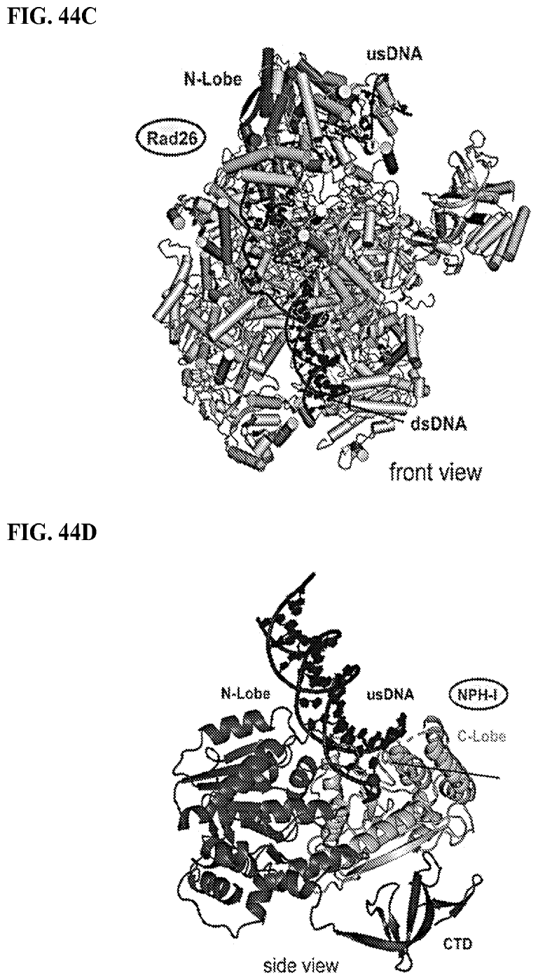

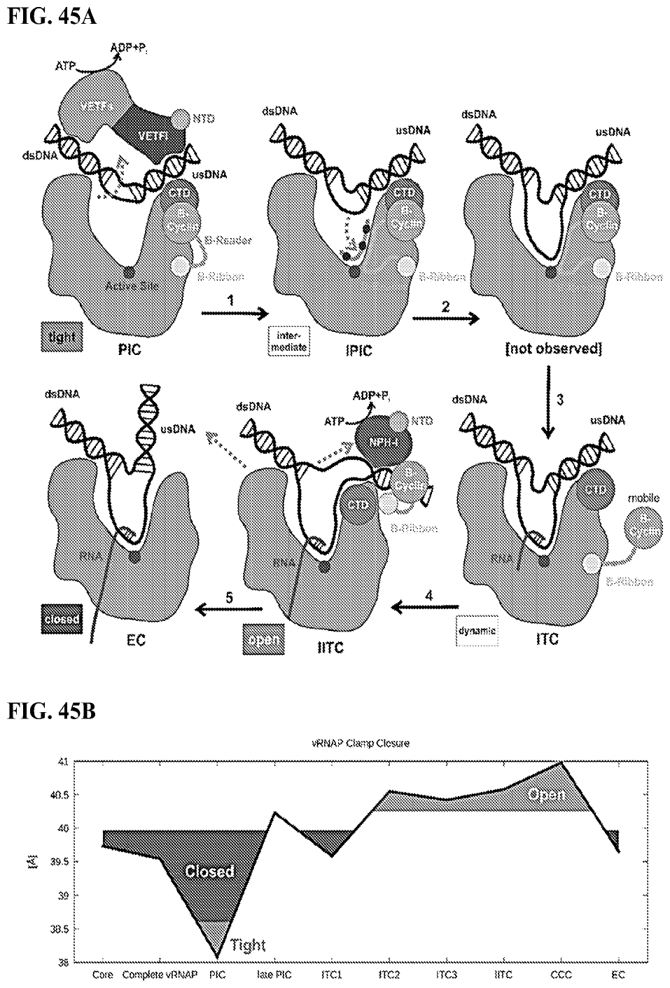

Three additional vRNAP particle classes yielded reconstructions that were assigned to different conformations of the ITC based on their composition and promotor position. The latter could be safely determined as the downstream blunt end of the synthetic promoter scaffold was readily visible in the density, even though its quality did not allow for identification of single bases. The different structures were termed ITC1, ITC2 and ITC3. In contrast to the 1PIC, ordered density was observed for DNA in the downstream DNA channel and for a DNA/RNA hybrid above the active site. Consequently, the Rpo30 PPD, which occupied the position of the DNA/RNA hybrid in the IPIC has been displaced by the template strand and the B homology region became mobile and is not visible in the density (FIG. 44B). No density for upstream DNA was identified. The three ITC complexes superimposed well but differed in the positioning of the DNA within the downstream DNA channel (FIG. 43) and the opening state of the clamp (FIG. 45B). For ITC3, the downstream DNA density was located in a shallower position and was less ordered compared to the other two. In the ITC1 and ITC2 particles, the clamp is in a closed conformation with the DNA bound firmly and deep in the downstream DNA channel. In contrast, ITC3 features the clamp in the open conformation and the promoter is mobile in a shallower position within the downstream DNA channel. No significant differences between the three ITC complexes were discernible with regard to the DNA/RNA hybrid region. Thus, the three ITC structures inform on the ITC's conformational flexibility and on the template-strand capture mechanism discussed below.

Structure of a Late Initially Transcribing Complex

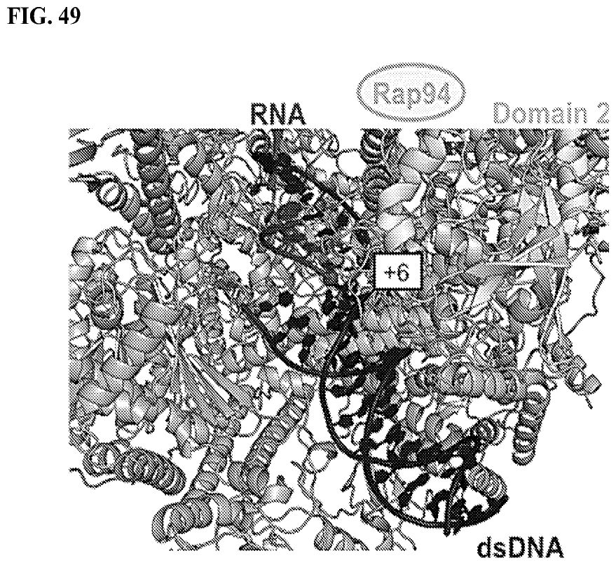

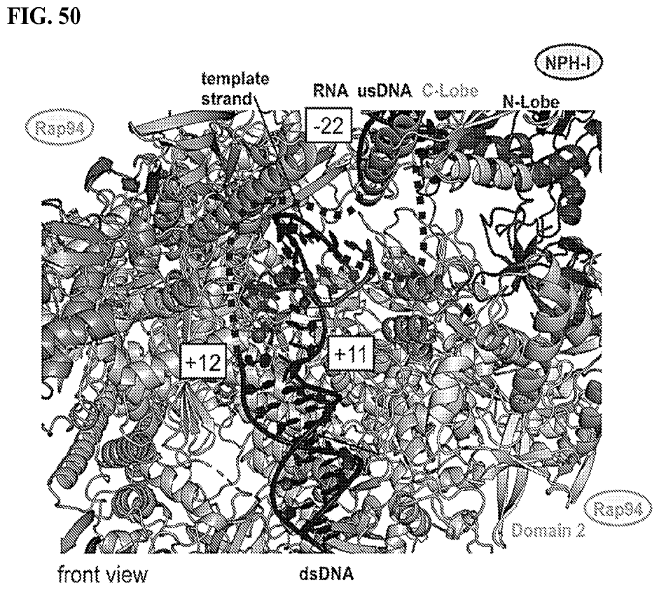

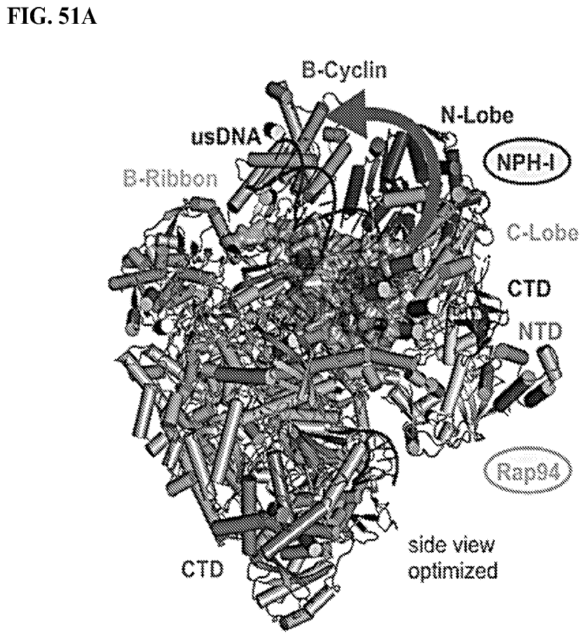

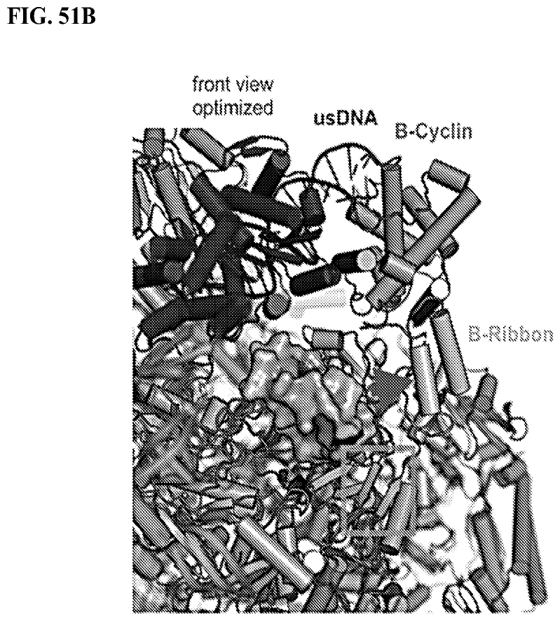

A particular class stood out because it belonged to a particle considerably larger than the ITC (FIG. 47a). After a further round of focused classification on the extra density followed by multibody refinement a reconstruction was obtained that allowed a complete modelling of the particle (FIG. 44, see also Extended Data FIG. 47b-47d and Materials and Methods for details). This complex is classified as a late form of the ITC (1ITC), primarily based on the positions of the blunt ends of the upstream and downstream promoter-DNA segments that are well visible in the density. Except for Rap94 and the RNA/DNA hybrid the core vRNAP was in a conformation similar to that observed in the ITC complexes and the downstream path of the DNA fitted best the ITC1 particle. The downstream blunt end of the DNA duplex indicated that the core vRNAP had advanced 5 bp compared to the situation in the ITC1-3 particles (compare FIG. 49 to FIG. 50).

In contrast, the other regions of the particle did not match any other known RNAP complex reported in the databank. The massive extra density above the cleft was identified as upstream DNA-bound NPH-I. In addition, Rap94 could unambiguously be located in the density. However, its B-homology region, the NTD as well as adjacent linkers appeared completely reconfigured in comparison to all other vRNAP complexes. It was also noted that the path of the upstream DNA in the 1ITC is fundamentally different from that observed in the PIC (see accompanying Example) and ITC (FIG. 43a).

While a databank search failed to identify any homologous RNAP complexes, it was noted that the helicase Rad26^{23,24} (human: CSB) in the structure of yeast Rad26-bound Pol II²² occupied a topologically equivalent position to NPH-I in the 1ITC, albeit in a different orientation (FIG. 44c). Furthermore, both complexes share the unique feature of a helicase-induced deflection of the DNA exit path by 80° at the upstream fork point of the transcription bubble²², albeit in different directions. Contrary to Rad26-bound Pol II, which lacks TFIIB, the vaccinia 1ITC still contains the TFIIB homologue Rap94, indicating a deviating functional role. It was concluded that the 1ITC is a unique viral complex which bears topological analogies to a functionally unrelated complex of Pol II, which is involved in transcription-coupled repair.

NPH-I is an Upstream Promoter Scrunching Motor

The blunt ends of the DNA promoter scaffold are clearly visible in the EM density of the 1ITC, thus allowing to determine the position of vRNAP relative to, and the size of, the transcription bubble. Compared to the ITC (FIG. 49), 5 bp of downstream DNA have been scrunched into the core vRNAP. Strikingly, also upstream of the artificial non-complementary region of the promoter scaffold 13 bp have additionally melted (FIG. 50). It was assumed that the NPH-I helicase motor^{12,31} delivers the free energy for this process by pulling the upstream DNA duplex into the core vRNAP, and simultaneously separating both strands. This results in an extremely large transcription bubble, reaching from promoter position +12 to −22. Downstream promoter scrunching occurs during Pol II initial transcription and promoter escape^{20,21,32}. The viral 1ITC appears to employ a unique mechanism in which downstream and upstream promoter scrunching is combined. By this means, the ATP-driven NPH-I may assists an effective promoter escape by the generation of a particularly energy-rich intermediate [Straney & Crothers]. This intermediate stores energy inside the large, melted transcription bubble, made readily available by re-annealing during the promoter escape reaction. NPH-I has been described as a positive transcription elongation facor^{12,27} and might act similarly when either recruited by a stalled vRNAP or as a component of the EC, analogous to CSB/RAD26 in the host polymerase system^{24,28}. Along these lines, NPH-I serves as a transcription elongation factor by increasing translation through T-rich sequences¹². In vivo, elongating vRNAP is associated with catalytically active NPH-I²⁷.

NPH-I may also orchestrates other processes necessary for promoter escape. When comparing the state of the distal upstream DNA in the ITC (FIG. 43) and EC²⁵ with that in the 1ITC (FIG. 44A, 44C) it is obvious that NPH-I has a strong ordering effect in this region. The 80° bend of the helix axis and the insertion of the ‘wedge’ residue Phe273 (FIG. 44D) stabilize the upper fork point of the transcription bubble of the 1ITC. For initiation of the eukaryotic transcription coupled repair process, it had been proposed that the helicase CSB/RAD26 pulls the template strand away from the polymerase, analogous to the action of SNF2 on chromatin^{22,28,29}. The similar architecture of the CSB/Rad26-PolII complex and the vaccinia 1ITC along with the fact that the helicases of both complexes belong to the SNF2 family, suggests that they also act mechanistically similar. However, a direct comparison of NPH-I to Rad26 also unravels important differences such as the lack of a brace helix in Rad26 and due to the section-wise disorder of the transcription bubble in the 1PIC the polarity of the NPH-I helicase motor cannot be conclusively determined.

A Comprehensive Model of the Initial Transcription Phase

Our vRNAP structures represent snapshots of states of the initiation phase of early gene transcription. Furthermore, the positioning of the polymerase on the promoter-DNA scaffold along with the nascent RNA allows a reliable assignment of the different states to the transcription timeline (FIG. 45A). In the PIC (see accompanying Example), vRNAP-bound VETF identified, aligned, positioned and melted the promoter DNA. Upon handover of the melted promoter to the core polymerase, VETF leaves the PIC and thus forms the 1PIC. In this complex, the upstream promoter is supported by the Rap94 CTD, the downstream portion is anchored in the downstream DNA channel (FIG. 45A, step 1, compare also accompanying Example). The single stranded DNA region is dynamic in this phase and therefore not visible (FIG. 42A). Through the interaction with the PPD of Rpo30 the B-homology domain of Rap94 is kept in an initiation-ready conformation. The template-strand capture goes along with the displacement of the PPD, which might be driven by the pronounced electronegative charge of the nucleic acid interacting with the positively charged active site region of vRNAP. After single strand capture, the B-reader may scan the template strand for the transcription start site (TSS) in an analogous manner as has been observed for Pol II (FIG. 45A, step 2). Once the TSS is located, the B-homology domain becomes mobile and RNA synthesis commences (FIG. 45A, step 3). This phase is highly dynamic as documented by three different ITC structures deviating in the state of the clamp (FIG. 45B) and positioning of the downstream DNA in the downstream DNA channel (FIG. 43A). The vRNAP promoter escape is accompanied by recruitment of NPH-I, a large-scale remodeling of Rap94, and major changes to the path of the upstream DNA (FIG. 45A, step 4). In the resulting 1ITC complex (FIG. 44A), the NPH-I evidently acts as a strand-separating helicase, widens the transcription bubble, defines its upstream fork point, and shapes the path of the single-stranded template and non-template DNA (FIG. 44C). Transition to a processive EC (FIG. 45, step 5) includes contraction of the transcription bubble, mobilization of the upstream DNA duplex and loss of NPH-I. In vitro, a processive vRNAP EC can be assembled in absence of Rap94²⁵, while in vivo, EC complexes are found associated with the latter^{33,35}. Here, Rap94 could ensure the efficient recruitment of NPH-I to ECs stalled at intrinsic pause sites to facilitate their readthrough in concert with NPH-I¹². It seems likely that the resultant vRNAP complex is structurally similar to the 1ITC (FIG. 44A).

Methods

vRNAP Purification from Recombinant Vaccinia Virus GLV-1h439

The generation of GLV-1h439 has been described previously¹⁹. For vRNAP purification, Hela S3 cells were cultured in Dulbecco's modified Eagle Medium (DMEM), containing 10% fetal bovine serum at 37° C. in a present of 5% CO₂. Cells were grown up to 80-90% of confluency and then infected with purified GLV-1h439 with a multiplicity of infection (MOI) of 1.2. After 24 h, the infected cells were pelleted and resuspended in lysis buffer (50 mM HEPES, pH 7.5, 150 mM NaCl, 1.5 mM MgCl₂, 0.5% [v/v] NP-40, 1 mM DTT, and complete EDTA-free protease inhibitor cocktail (Sigma-Aldrich). The soluble supernatant of the cellular extract was incubated for 3h at 4° C. with anti-FLAG Agarose beads (Sigma Aldrich). Beads were washed four times with buffer containing 50 mM HEPES, pH 7.5, 150 mM NaCl, 1.5 mM MgCl₂, 0.1% [v/v] NP-40, 1 mM DTT, equilibrated with elution buffer (50 mM HEPES, pH 7.5, 150 mM NaCl, 1.5 mM MgCl₂ and 1 mM DTT) and eluted with a 200 μg/ml solution of 3×FLAG Peptide (Sigma-Aldrich). The eluate was analyzed by SDS-PAGE and the protein components were identified by mass spectrometry (see also FIG. 46b). Approx. 50 μg of purified vRNAP was obtained from one 15 cm petri-dish of Hela S3 cells infected with the virus.

Reconstitution of Promoter Bound vRNAP Complexes

A synthetic double stranded DNA oligonucleotide scaffold mimicking the vaccinia virus early promoter region was generated by annealing of two partially complementary DNA oligonucleotides (see FIG. 46a). Annealing was performed in buffer containing 100 mM NaCl, 20 mM HEPES, pH 7.5, and 3 mM MgCl₂ by heating the mixture to 95° C. for 5 min followed by slowly cooling down to room temperature. The resulting double stranded DNA oligo was precipitated by isopropanol and the dry pellet was resuspended in 1× resuspension buffer (10 mM Tris-HCl, pH 8.0, 1 mM EDTA).

For reconstitution of promoter-bound vRNAP complexes approx. 1 pmol of [³²P]-labeled DNA promoter-scaffold was incubated for 30 min at 30° C. with the indicating amount of vRNAP in the presence of 1 mM of the indicated NTPs (FIG. 46). Reconstitutions were analyzed by native gel electrophoresis (4% acrylamide and 0.13% bis-acrylamide, 25 mM Tris-HCl pH 7.4, 25 mM Boric acid and 0.5 mM EDTA) at 4° C. For large-scale reconstitution of promoter/vRNAP complexes, purified vRNAP was concentrated in a Viva-spin (Sartorius). A total of 400 μg of vRNAP was incubated with a 60 fold-molar excess of the DNA scaffold in reconstitution buffer (50 mM NaCl, 10 mM Tris-HCl, pH 7.5, 5 mM MgCl₂ and 1 mM DTT) in the presence of ATP and UTP (1 mM each) for 30 min at 30° C. The mixture was separated by 10%-30% sucrose gradient centrifugation (16h, 35.000 rpm, Beckman 60Ti rotor, 4° C.). Gradient fractions were collected manually and analyzed by SDS-PAGE followed by Silver staining and ethidium-bromide staining to visualize the proteins and the DNA scaffold, respectively. The indicated fractions (FIG. 46) were used for cryo-EM analysis after buffer exchange with modified reconstitution buffer (100 mM NaCl, 10 mM Tris-HCl, pH 7.5, 5 mM MgCl₂ and 1 mM DTT) in a Vivaspin concentrator (Sartorious; 10 MW cut-off).

Transcription Assay

A plasmid containing an early vaccinia virus promoter fused with a G-less cassette (termed psB24) was used. For vRNAP catalyzed transcription assays, 400 ng of SmaI-linearized pSB24 template was incubated with 100 μg vRNAP in buffer containing 40 mM Tris-HCl, pH 7.9, 1 mM DTT, 2 mM spermidine, 6 mM MgCl₂, 1 mM ATP, CTP and GTP, 0.1 mM UTP and 20 μCi [³²P]-UTP, and 80 μM S-adenosyl-methionine. The transcription mixture was incubated at 30° C. for the indicated time points. Radio-labelled RNA transcripts were tryzol-extracted, precipitated by isopropanol and analyzed by denaturing 5% urea polyacrylamide gel electrophoresis. Transcripts were subsequent visualized by autoradiography.

Cryo-Electron Microscopic Data Collection and Initial Data Processing

Following sucrose gradient purification, the indicated fractions (see FIG. 46) was diluted 1:50 with a buffer containing 10 mM Tris-HCl, pH 7.5, 100 mM NaCl, 5 mM MgCl₂ and 1 mM DTT, and centrifuged in a Vivaspin concentrator to remove the sucrose. For cryo-EM analysis the sample was centrifuged for 40 min at 10,000 rpm. For cryo-EM data collection, R 1.2/1.3 holey carbon grids (Quantifoil) were glow-discharged for 90 s (Plasma Cleaner model PDC-002. Harrick Plasma Ithaca, N.Y./USA) at medium power, and 3.5 μl of C2 sample was applied inside a Vitrobot Mark IV (FEI) at 4° C. and 100% relative humidity. Grids were blotted for 3 s and with blot force 5 and plunged into liquid ethane. Cryo-EM datasets comprising 10816 (dataset 1), 9878 (dataset 2), and 3640 (dataset 3) micrographs, respectively, were collected from three different grids with a Thermo Fisher Titan Krios G3 and a Falcon III camera (Thermo-Fischer). Data was acquired with EPU at 300 keV and a primary magnification of 75,000 (calibrated pixel size 1.0635 Å) in movie-mode with 47 fractions per movie and counting of the electron signal. The total exposure was 77.5 e/Å² for 75 sec, with 2 exposures per hole.

Dose-weighted, motion-corrected sums of the micrograph movies were calculated with Motioncorr2 (Zheng et al., 2017). The contrast-transfer function of each micrograph was fitted with CTFFind4 (Rohou and Grigorieff, 2015). An initial set of 25,000 particles was picked with the Gaussian picker and subjected to three rounds of 2D-Classification in Relion3.1 (Zivanov et al., 2018) to clean up the dataset. 8 reasonable class averages were selected as templates for subsequent automated particle picking within Relion and a total of 300,000 particles were picked using the Relion autopicker. After a second round of 2D classification, 3D classification was performed using the vRNAP core structure as template. Particles belonging to the ITC and 1ITC classes, each, were selected and 2D classes for picking of 1ITC and ITC particles, respectively, were calculated. The resulting 1ITC and ITC 2D classes served as autopicking templates to extract a separate particle stack for ITC (FIG. 46A) and 1ITC (FIG. 47A) from each of the three full datasets. The resulting six particle stacks were cleaned up by four rounds of 2D classification, each, and contained 1,513,003 (dataset 1), 924,405 (dataset 2), and 323,776 (dataset 3) good particles for ITC, 1,062,912 (dataset 1), 942,258 (dataset 2), and 323,776 (dataset 3) good particles for 1ITC. Each of the particle stacks was then subjected to 3D classification and particles belonging to the appropriate ITC or 1ITC classes were selected. The three ITC particle stacks of the three datasets were then united into a single stack, and CTF refinement followed by a consensus 3D refinement was performed. The same was done for 1ITC.

3D Reconstruction and Model Building of 1PIC and ITC Complexes

The 1PIC particle stack obtained as described above was subjected to two rounds of focused 3D classification with 3 classes in each of the two rounds. The classification was focused with a mask on the cleft, active site and downstream DNA channel as well as the region of the Rap94 cyclin domain. From the resulting set of nine class averages (FIG. 46A) four reasonable reconstructions were obtained after a final round of 3D refinement and post-processing, and the associated complexes were identified as the 1PIC, and ITC1-3 (FIG. 46B). The resolution was determined by fourier-shell correlation (FSC) to 2.99 Å for the 1PIC and 2.88 Å, 3.15 Å and 3.04 Å for ITC1, ITC2 and ITC3, respectively (FIG. 46C). To build the 1PIC model, the vRNAP core including the Rpo30 PPD was extracted from the complete vRNAP structure (PDB 6RFL) and docked into the cryo EM density. The positioning of the Rap94 cyclin domain and the adjacent linker regions were adjusted manually with Coot and the model was refined with Phenix.real_space_refine including an ADP refinement step. During refinement secondary structure and mild Ramachandran restraints were imposed. After two further cycles of manual inspection and automated refinement, the refinement converged and a model with excellent stereochemistry and good correlation with the cryo EM map was obtained.

3D Reconstruction and Model Building of 1ITC

The 1ITC particle stack obtained as described above was subjected to a round of focused 3D classification with a mask on the NPH-I and upstream DNA region. From the three resulting classes, a single one displayed good occupancy and resolution for NPH-I. Particles belonging to this class were subjected to a two-body multibody refinement (MB) in Relion using a mask for NPH-I and upstream DNA and a mask for the core vRNAP. The postprocessed reconstructions for both bodies were then combined with Phenix.combine_focused_maps. To build the 1ITC model, the ITC1 structure was docked into the density. Within the residual density a characteristical SNF2 helicase fold was recognized that was docked with either VETFs (see accompanying Example) or NPH-I from the complete vRNAP structure (PDB 6RFL). NPH-I unequivocally fitted the density while VETFs did not. Further residual density could then be identified as the relocated Rap94 B cyclin domain the relocated Rap94 NTD and the NPH-I CTD. After manual adjustments with Coot including rebuilding of remodeled Rap94 linker regions the model was refined with Phenix.real_space_refine including an ADP refinement step. During refinement secondary structure and mild Ramachandran restraints were imposed. After two further cycles of manual inspection and automated refinement, the refinement converged and a model with excellent stereochemistry and good correlation with the cryo EM map was obtained.

### REFERENCES FOR EXAMPLE 5

1. Broyles, S. S. & Moss, B. Homology between RNA polymerases of poxviruses, prokaryotes, and eukaryotes: nucleotide sequence and transcriptional analysis of vaccinia virus genes encoding 147-kDa and 22-kDa subunits. Proc Natl Acad Sci USA 83, 3141-5 (1986).
2. Patel, D. D. & Pickup, D. J. The second-largest subunit of the poxvirus RNA polymerase is similar to the corresponding subunits of procaryotic and eucaryotic RNA polymerases. J Virol 63, 1076-86 (1989).
3. Amegadzie, B. Y. et al. Identification, sequence, and expression of the gene encoding the second-largest subunit of the vaccinia virus DNA-dependent RNA polymerase. Virology 180, 88-98 (1991).
4. Amegadzie, B. Y., Ahn, B. Y. & Moss, B. Identification, sequence, and expression of the gene encoding a Mr 35,000 subunit of the vaccinia virus DNA-dependent RNA polymerase. J Biol Chem 266, 13712-8 (1991).
5. Ahn, B. Y., Gershon, P. D., Jones, E. V. & Moss, B. Identification of rpo30, a vaccinia virus RNA polymerase gene with structural similarity to a eucaryotic transcription elongation factor. Mol Cell Biol 10, 5433-41 (1990).
6. Broyles, S. S. & Pennington, M. J. Vaccinia virus gene encoding a 30-kilodalton subunit of the viral DNA-dependent RNA polymerase. J Virol 64, 5376-82 (1990).
7. Ahn, B. Y., Rosel, J., Cole, N. B. & Moss, B. Identification and expression of rpo19, a vaccinia virus gene encoding a 19-kilodalton DNA-dependent RNA polymerase subunit. J Virol 66, 971-82 (1992).
8. Ahn, B. Y., Jones, E. V. & Moss, B. Identification of the vaccinia virus gene encoding an 18-kilodalton subunit of RNA polymerase and demonstration of a 5′ poly(A) leader on its early transcript. J Virol 64, 3019-24 (1990).
9. Quick, S. D. & Broyles, S. S. Vaccinia virus gene D7R encodes a 20,000-dalton subunit of the viral DNA-dependent RNA polymerase. Virology 178, 603-5 (1990).
10. Amegadzie, B. Y., Ahn, B. Y. & Moss, B. Characterization of a 7-kilodalton subunit of vaccinia virus DNA-dependent RNA polymerase with structural similarities to the smallest subunit of eukaryotic RNA polymerase II. J Virol 66, 3003-10 (1992).
11. Ahn, B. Y. & Moss, B. RNA polymerase-associated transcription specificity factor encoded by vaccinia virus. Proc Natl Acad Sci USA 89, 3536-40 (1992).
12. Deng, L. & Shuman, S. Vaccinia NPH-I, a DExH-box ATPase, is the energy coupling factor for mRNA transcription termination. Genes Dev 12, 538-46 (1998).
13. Shuman, S. Vaccinia virus RNA helicase: an essential enzyme related to the DE-H family of RNA-dependent NTPases. Proc Natl Acad Sci USA 89, 10935-9 (1992).
14. Baldick, C. J., Jr., Cassetti, M. C., Harris, N. & Moss, B. Ordered assembly of a functional preinitiation transcription complex, containing vaccinia virus early transcription factor and RNA polymerase, on an immobilized template. J Virol 68, 6052-6 (1994).
15. Ahn, B. Y., Gershon, P. D. & Moss, B. RNA polymerase-associated protein Rap94 confers promoter specificity for initiating transcription of vaccinia virus early stage genes. J Biol Chem 269, 7552-7 (1994).
16. De la Pena, M., Kyrieleis, O. J. & Cusack, S. Structural insights into the mechanism and evolution of the vaccinia virus mRNA cap N7 methyl-transferase. EMBO J26, 4913-25 (2007).
17. Shuman, S., Broyles, S. S. & Moss, B. Purification and characterization of a transcription termination factor from vaccinia virions. J Biol Chem 262, 12372-80 (1987).
18. Shuman, S. & Hurwitz, J. Mechanism of mRNA capping by vaccinia virus guanylyltransferase: characterization of an enzyme—guanylate intermediate. Proc Natl Acad Sci USA 78, 187-91 (1981).
19. Grimm, C. et al. Structural Basis of Poxvirus Transcription: Vaccinia RNA Polymerase Complexes. Cell 179, 1537-1550 e19 (2019).
20. Revyakin, A., Liu, C., Ebright, R. H. & Strick, T. R. Abortive initiation and productive initiation by RNA polymerase involve DNA scrunching. Science 314, 1139-43 (2006).
21. Kapanidis, A. N. et al. Initial transcription by RNA polymerase proceeds through a DNA-scrunching mechanism. Science 314, 1144-7 (2006).
22. Xu, J. et al. Structural basis for the initiation of eukaryotic transcription-coupled DNA repair. Nature 551, 653-657 (2017).
23. Duan, M., Selvam, K., Wyrick, J. J. & Mao, P. Genome-wide role of Rad26 in promoting transcription-coupled nucleotide excision repair in yeast chromatin. Proc Natl Acad Sci USA 117, 18608-18616 (2020).
24. Ghosh-Roy, S., Das, D., Chowdhury, D., Smerdon, M. J. & Chaudhuri, R. N. Rad26, the transcription-coupled repair factor in yeast, is required for removal of stalled RNA polymerase-II following UV irradiation. PLoS One 8, e72090 (2013).
25. Hillen, H. S. et al. Structural Basis of Poxvirus Transcription: Transcribing and Capping Vaccinia Complexes. Cell 179, 1525-1536 e12 (2019).
26. Christen, L. M., Sanders, M., Wiler, C. & Niles, E. G. Vaccinia virus nucleoside triphosphate phosphohydrolase I is an essential viral early gene transcription termination factor. Virology 245, 360-71 (1998).
27. Piacente, S., Christen, L., Dickerman, B., Mohamed, M. R. & Niles, E. G. Determinants of vaccinia virus early gene transcription termination. Virology 376, 211-24 (2008).
28. Svejstrup, J. Q. Rescue of arrested RNA polymerase II complexes. J Cell Sci 116, 447-51 (2003).
29. Saha, A., Wittmeyer, J. & Cairns, B. R. Chromatin remodeling by RSC involves ATP-dependent DNA translocation. Genes Dev 16, 2120-34 (2002).
30. Burton, S. P. & Burton, Z. F. The sigma enigma: bacterial sigma factors, archaeal TFB and eukaryotic TFIIB are homologs. Transcription 5, e967599 (2014).
31. Hindman, R. & Gollnick, P. Nucleoside Triphosphate Phosphohydrolase I (NPH I) Functions as a 5′ to 3′ Translocase in Transcription Termination of Vaccinia Early Genes. J Biol Chem 291, 14826-38 (2016).
32. Roberts, J. W. Biochemistry. RNA polymerase, a scrunching machine. Science 314, 1097-8 (2006).
33. Yang, Z. & Moss, B. Interaction of the vaccinia virus RNA polymerase-associated 94-kilodalton protein with the early transcription factor. J Virol 83, 12018-26 (2009).
34. Tate, J. & Gollnick, P. Role of forward translocation in nucleoside triphosphate phosphohydrolase I (NPH I)-mediated transcription termination of vaccinia virus early genes. J Biol Chem 286, 44764-75 (2011).
35. Zhang, Y., Ahn, B. Y. & Moss, B. Targeting of a multicomponent transcription apparatus into assembling vaccinia virus particles requires RAP94, an RNA polymerase-associated protein. J Virol 68, 1360-70 (1994).

### Example 6. Potential Inhibitors

A list of potential inhibitors which show differential effects is provided in Table 4 below. Compound stocks were prepared at a concentration of 1 mM in 65% DMSO, at a volume of 5 μL each. In an embodiment, the inhibitor is any one of the compounds listed in Table 4.

TABLE 4
Potential inhibitors, percent inhibition, and chemical structure.

| | Results Screen | | | |
|---|---|---|---|---|
| | Median | | Results Hit | |
| | Screen | | validation | |
| | (% inhi- | IQR (% | Median (% | |
| Cpd ID | bition) | inhibition) | inhibition) | Structure and Chirality |
| AD153413- 01_A10 | 84.5 | 46 | 77.6 | 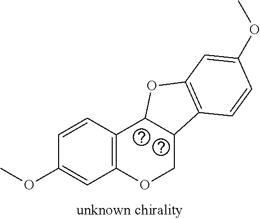 [SMILES: COc1ccc2c(c1)OC1c3ccc(OC)cc3OCC21] |
| AD153413- 01_B11 | 75.6 | 70.9 | 72.1 | 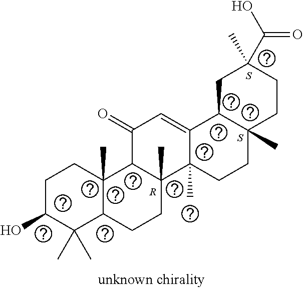 |
| AD153413- 02_F8 | 86.7 | 22.3 | 44 | 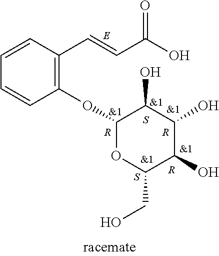 |
| AD153413- 03_F11 | 70.6 | 66.7 | 41.8 | 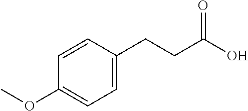 |
| AD153413- 03_H2 | 73.1 | 10.6 | 48.9 | 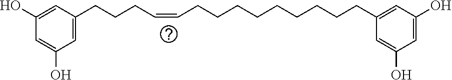 [SMILES: Oc1cc(O)cc(CCC/C=C\CCCCCCCCCc2cc(O)cc(O)c2)c1] |
| AD153413- 04_H6 | 77 | 17.3 | 52.2 | 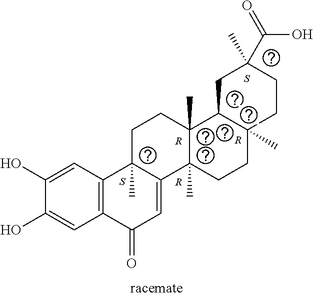 |
| AD153413- 05_B3 | 72.4 | 91.8 | 43.6 | 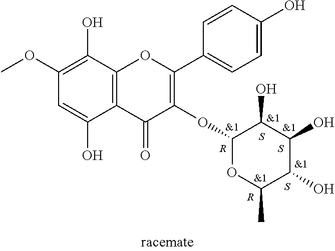 |
| AD153413- 06_A10 | 72.4 | 15.7 | 64.1 | 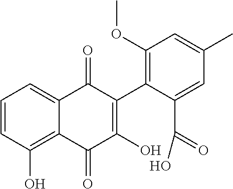 [SMILES: COc1cc(C)cc(C(=O)O)c1C1=C(O)C(=O)c2c(O)cccc2C1=O] |
| AD153413- 06_B9 | 80 | 15.5 | 42.9 | 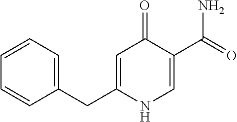 [SMILES: NC(=O)c1c[nH]c(Cc2ccccc2)cc1=O] |
| AD153413- 07_H11 | 70 | 39.7 | 45.2 | 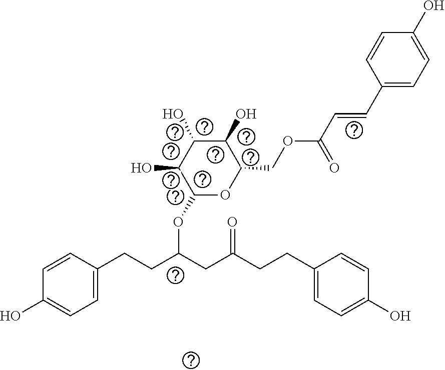 |
| AD153413- 08_B4 | 73.6 | 28.2 | 41.9 | 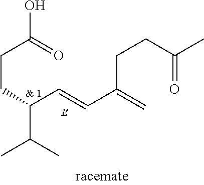 |
| AD153413- 09_A3 | 89.7 | 24 | 55.9 | 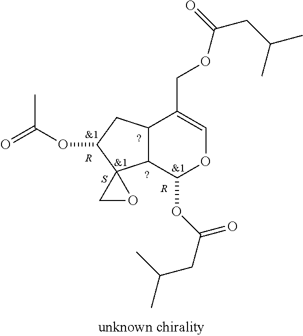 |
| AD153413- 09_A6 | 81.1 | 61.4 | 64.2 | 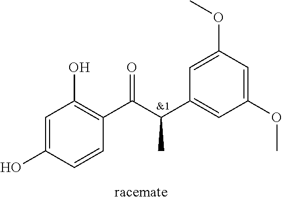 |
| AD153413- 09_B9 | 85.2 | 13.7 | 61.5 | 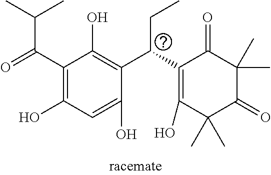 [SMILES: CC[C@H](C1=C(O)C(C)(C)C(=O)C(C)(C)C1=O)c1c(O)cc(O)c(C(=O)C(C)C)c1O] |
| AD153413- 09_E2 | 90.3 | 24.1 | 60.5 | 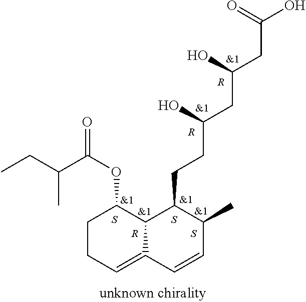 |
| AD153413- 10_B3 | 60.9 | 73.9 | 43.3 | 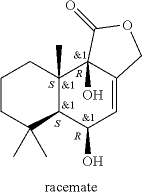 |
| AD153413- 10_F2 | 80 | 65.7 | 50.8 | 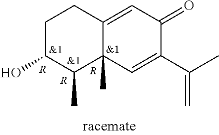 |
| AD153413- 11_A11 | 83 | 57 | 57.1 | 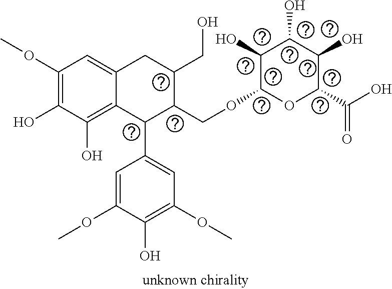 [SMILES: COc1cc(C2c3c(cc(OC)c(O)c3O)CC(CO)C2CO[C@H]2O[C@@H](C(=O)O)[C@H](O)[C@@H](O)[C@@H]2O)cc(OC)c1O] |
| AD153413- 11_H9 | 60.5 | 34.6 | 54.9 | 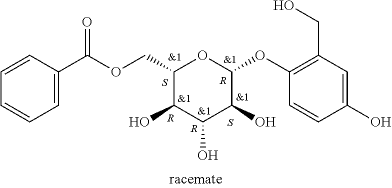 |
| AD153413- 12_C11 | 61.7 | 43.4 | 44.2 | 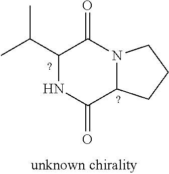 |
| AD153413- 12_D11 | 73.7 | 9.2 | 50.1 | 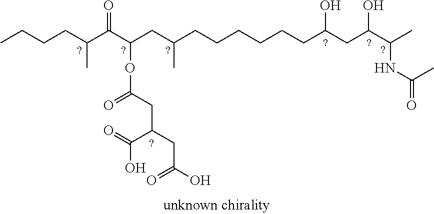 |
| AD153413- 14_C5 | 63.3 | 8.2 | 52.6 | 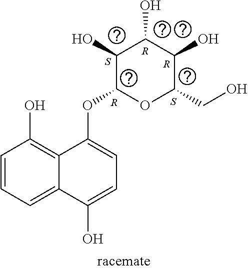 |
| AD153413- 14_E9 | 67.1 | 26.8 | 43.8 | 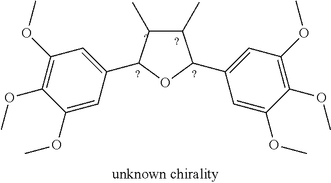 |
| AD153413- 16_A3 | 83 | 39.6 | 44.5 | 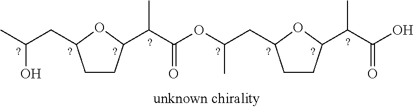 |
| AD153413- 16_B4 | 72.5 | 65.2 | 53.6 | 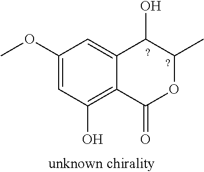 |
| AD153413- 16_C3 | 84.3 | 31.7 | 40.5 | 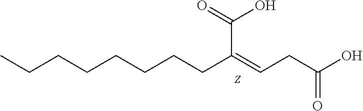 |
| AD153413- 16_E3 | 77.7 | 107.7 | 55.9 | 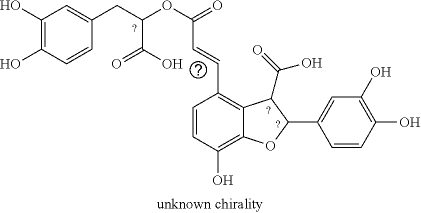 [SMILES: O=C(/C=C/c1ccc(O)c2c1C(C(=O)O)C(c1ccc(O)c(O)c1)O2)OC(Cc1ccc(O)c(O)c1)C(=O)O] |
| AD153413- 16_G9 | 87.4 | 37.6 | 64.7 | 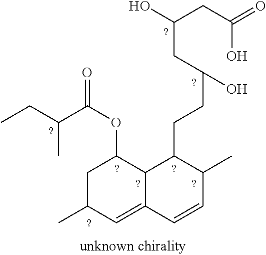 |
| AD153413- 17_A4 | 69.6 | 28.4 | 52.3 | 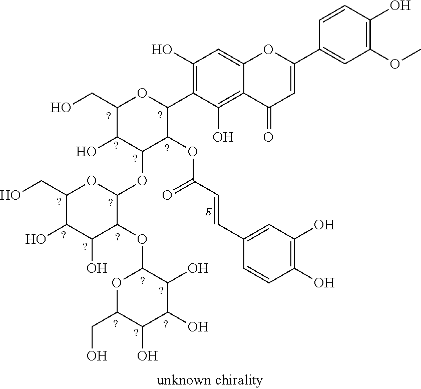 |
| AD153413- 17_A5 | 71 | 65.8 | 70.9 | 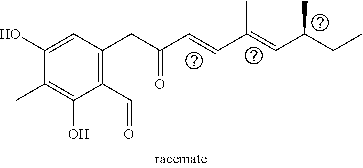 [SMILES: CC[C@H](C)/C=C(C)/C=C/C(=O)Cc1cc(O)c(C)c(O)c1C=O] |
| AD153413- 17_A6 | 77.1 | 31.9 | 62.2 | 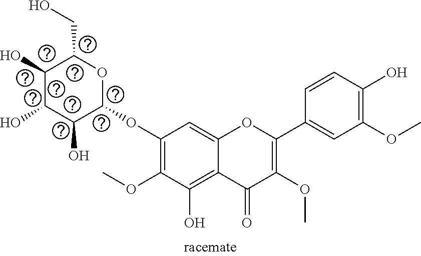 [SMILES: COc1cc(-c2oc3cc(O[C@H]4O[C@@H](CO)[C@H](O)[C@@H](O)[C@@H]4O)c(OC)c(O)c3c(=O)c2OC)ccc1O] |
| AD153413- 18_A11 | 81 | 67.4 | 60.6 | 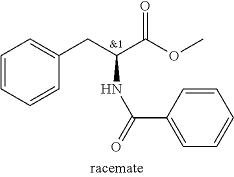 |
| AD153413- 19_D3 | 71.8 | 78.9 | 41.6 | 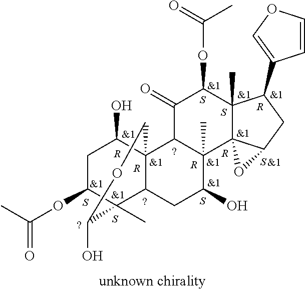 |
| AD153413- 19_H4 | 98.8 | 67.1 | 50.8 | 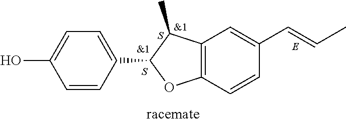 |
| AD153413- 21_A6 | 68.8 | 19.2 | 54.7 | 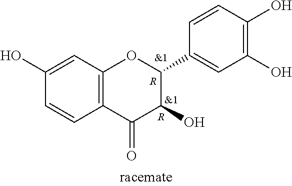 |
| AD153413- 23_G3 | 60.5 | 26.2 | 61.7 | 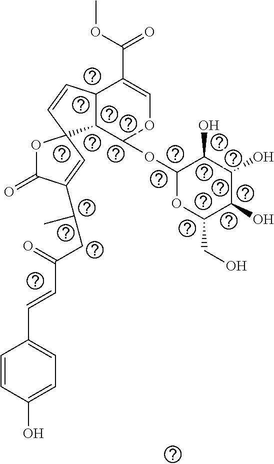 |
| AD153413- 25_A7 | 85.9 | 20.7 | 70 | 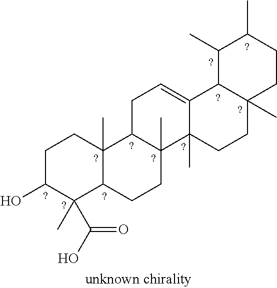 |
| AD153413- 25_A8 | 73.8 | 10 | 86.3 | 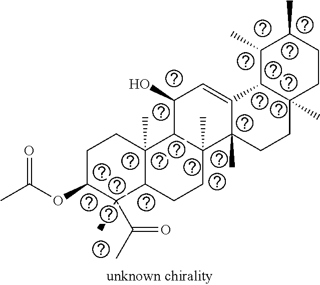 |
| AD153413- 25_E5 | 72.1 | 11.5 | 56 | 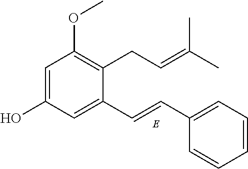 |
| AD153413- 25_H9 | 79.1 | 8.8 | 51.1 | 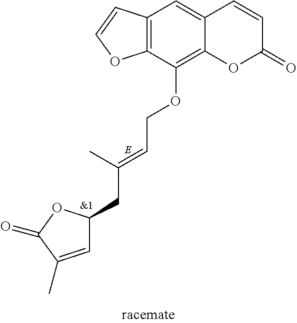 |

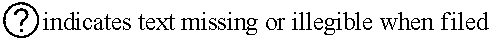

What is claimed is:

1. A method for regulating activity of a poxvirus viral polymerase in a cell infected with the poxvirus, the method comprising contacting the cell with a compound that reduces or prevents interaction of the viral polymerase with a glutamine tRNA (tRNA^{Glu}).

2. The method of claim 1, wherein the tRNA^{Glu} is an uncharged tRNA^{Glu}.

3. The method of claim 1 or 2, wherein the poxvirus is a variola virus or variant thereof.

4. The method of any one of claims 1 to 3, wherein the compound comprises a small molecule, an antisense RNA, an antibody, an aptamer, or a polypeptide.

5. The method of any one of claims 1 to 4, wherein the viral polymerase is a virus-encoded RNA polymerase.

6. The method of claim 5, wherein the viral polymerase is a virus-encoded multisubunit RNA polymerase (vRNAP).

7. The method of any one of claims 1 to 6, wherein the cell is a stem cell, immune cell, or cancer cell.

8. The method of claim 7, wherein the stem cell is selected from adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, and combinations thereof.

9. A method for treating or preventing infection by poxvirus in a subject in need thereof, wherein the poxvirus comprises a viral polymerase, the method comprising administering to the subject a compound that reduces or prevents interaction of the viral polymerase with a glutamine tRNA (tRNA^{Glu}).

10. The method of claim 9, wherein the tRNA^{Glu} is an uncharged tRNA^{Glu}.

11. The method of claim 9 or 10, wherein the poxvirus is a variola virus or variant thereof.

12. The method of any one of claims 9 to 11, wherein the compound comprises a small molecule, an antisense RNA, an antibody, an aptamer, or a polypeptide.

13. The method of any one of claims 9 to 12, wherein the viral polymerase is a virus-encoded RNA polymerase.

14. The method of claim 13, wherein the viral polymerase is a virus-encoded multisubunit RNA polymerase (vRNAP).

15. A method for modulating activity of a poxvirus viral polymerase in a cell infected with the poxvirus, the method comprising contacting the cell with glutamine, wherein the glutamine modulates interaction of the viral polymerase with a glutamine tRNA (tRNA^{Glu}).

16. The method of claim 15, wherein the poxvirus is a variola virus or variant thereof.

17. The method of claim 15, wherein the poxvirus is a vaccinia virus or variant thereof.

18. The method of claim any one of claims 15 to 17, wherein the glutamine reduces or prevents interaction of the viral polymerase with the tRNA^{Glu}.

19. The method of claim any one of claims 15 to 17, wherein the glutamine increases or promotes interaction of the viral polymerase with the tRNA^{Glu}.

20. The method of claim any one of claims 15 to 19, wherein the viral polymerase is a virus-encoded RNA polymerase.

21. The method of claim 20, wherein the viral polymerase is a virus-encoded multisubunit RNA polymerase (vRNAP).

22. The method of any one of claims 15 to 21, wherein the tRNA^{Glu} is an uncharged tRNA^{Glu}.

23. The method of any one of claims 15 to 22, wherein the cell is a stem cell, immune cell, or cancer cell.

24. The method of claim 23, wherein the stem cell is selected from adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, and combinations thereof.

25. A method for regulating activity of a poxvirus viral polymerase in a cell infected with the poxvirus, the method comprising contacting the cell with a compound that modulates activity of the viral polymerase.

26. The method of claim 25, wherein the compound reduces or inhibits activity of the viral polymerase.

27. The method of claim 25, wherein the compound enhances or promotes activity of the viral polymerase.

28. A method for treating or preventing infection by poxvirus in a subject in need thereof, wherein the poxvirus comprises a viral polymerase, the method comprising administering to the subject a compound that interacts with the active site of the viral polymerase.

29. The method of any one of claims 25 to 28, wherein the compound interacts with an active site of the viral polymerase.

30. The method of claim 29, wherein the active site comprises a binding site for a catalytic metal ion.

31. The method of claim 30, wherein the binding site is a D×D×D site on an Rpo147 subunit.

32. The method of claim 30 or 31, wherein the compound reduces or inhibits binding of the catalytic metal ion to the binding site for the catalytic metal ion.

33. The method of any one of claims 25 to 32, wherein the compound reduces or inhibits interaction of subunit Rpo30 with the active site.

34. The method of any one of claims 25 to 28, wherein the compound interacts with an active site of a poxvirus capping enzyme.

35. The method of any one of claims 25 to 28, wherein the compound inhibits or reduces interaction of one or more subunits of the viral polymerase from interacting with the viral polymerase.

36. The method of claim 35, wherein the one or more subunits of the viral polymerase comprise one or more of: Rpo147, Rpo132, Rpo35, Rpo22, Rpo19, Rpo18, Rpo7, Rpo30, Rap94, a capping enzyme, a termination factor, VETF-1, VETF-s, E11L, tRNA^{Glu}, NPH-1, VTF/CE, and/or any poxvirus polymerase subunit as listed or described in Appendix A and/or Appendix B, or a variant or homologue thereof.

37. The method of any one of claims 25 to 36, wherein the poxvirus is a variola virus or variant thereof.

38. The method of any one of claims 25 to 36, wherein the poxvirus is a vaccinia virus or variant thereof.

39. The method of any one of claims 25 to 38, wherein the viral polymerase is a virus-encoded RNA polymerase.

40. The method of claim 39, wherein the viral polymerase is a virus-encoded multisubunit RNA polymerase (vRNAP).

41. The method of any one of claims 25 to 40, wherein the compound comprises a small molecule, an antisense RNA, an antibody, an aptamer, or a polypeptide.

42. The method of any one of claims 1 to 41, wherein an RNA polymerase expressed by the infected cell or subject is not affected by the compound.

43. The method of any one of claims 1 to 42, wherein the subject is or the cell is from a mammal.

44. The method of claim 43, wherein the mammal is a human.

45. The method of any one of the above claims, wherein the compound that reduces or prevents interaction of the viral polymerase with a tRNA^{Glu} is a compound listed in Table 4.
